SEQUENCE LISTING (110 sequences)

SEQ ID NO: 1 - protein, 52 aa, Spinacia oleracea
  mat_peptide (1)..(52): SoD2 peptide
GIFSSRKCKTPSKTFKGICTRDSNCDTSCRYEGYPAGDCKGIRRRCMCSKPC

SEQ ID NO: 2 - protein, 38 aa, Spinacia oleracea
  mat_peptide (1)..(38): SoD7 peptide
GIFSSRKCKTPSKTFKGYCTRDSNCDTSCRYEGYPAGD

SEQ ID NO: 3 - DNA, 162 bp, Artificial Sequence
  Description of Artificial Sequence Synthetic SoD2 codon-optimized with GenScript
ggtattttctcatctaggaagtgcaaaactccttcaaagacttttaagggaatttgcactagggattctaattgcgatacttcttgcagatacgagggatatccagctggcgattgcaaaggaattaggaggagatgtatgtgttcaaagccatgttaataa

SEQ ID NO: 4 - DNA, 120 bp, Artificial Sequence
  Description of Artificial Sequence Synthetic SoD7 codon-optimized with GenScript
ggaattttctcttcaaggaagtgcaagactccatctaagactttcaagggatattgtactagggattctaactgcgatacatcatgcagatacgagggctatcctgctggcgattaataa

SEQ ID NO: 5 - DNA, 162 bp, Artificial Sequence
  Description of Artificial Sequence Synthetic SoD2 codon-optimized with CODA
ggtatcttttctagtagaaagtgtaagactccttctaagacttttaaaggtatttgcactagagattctaattgtgacacttcttgtagatatgaaggttatcctgctggtgattgtaagggtattagaagaagatgtatgtgttctaagccttgttaatag

SEQ ID NO: 6 - DNA, 120 bp, Artificial Sequence
  Description of Artificial Sequence Synthetic SoD7 codon-optimized with CODA
ggtattttttcatctcgtaagtgtaagactccttctaagacttttaagggttattgcactagagattctaattgtgatacatcttgtagatatgaaggttatcctgctggtgattaatag

SEQ ID NO: 7 - protein, 82 aa, Artificial Sequence
  Description of Artificial Sequence Synthetic chimeric peptide comprising a signal peptide and SoD2
MGFFLFSQMPSFFLVSTLLLFLIISHSSHAGIFSSRKCKTPSKTFKGICTRDSNCDTSCRYEGYPAGDCKGIRRRCMCSKPC

SEQ ID NO: 8 - protein, 68 aa, Artificial Sequence
  Description of Artificial Sequence Synthetic chimeric peptide comprising a signal peptide and SoD7
MGFFLFSQMPSFFLVSTLLLFLIISHSSHAGIFSSRKCKTPSKTFKGYCTRDSNCDTSCRYEGYPAGD

SEQ ID NO: 9 - DNA, 268 bp, Artificial Sequence
  Description of Artificial Sequence Synthetic chimeric nucleic acid encoding a signal peptide and SoD2 codon-optimized with GenScript
tctagaaacaatgggcttcttccttttctctcaaatgccttcatttttccttgtttctactcttcttctttttcttattatttctcattcttctcatgctggtattttctcatctaggaagtgcaaaactccttcaaagacttttaagggaatttgcactagggattctaattgcgatacttcttgcagatacgagggatatccagctggcgattgcaaaggaattaggaggagatgtatgtgttcaaagccatgttaataatctaga

SEQ ID NO: 10 - DNA, 226 bp, Artificial Sequence
  Description of Artificial Sequence Synthetic chimeric nucleic acid encoding a signal peptide and SoD7 codon-optimized with GenScript
tctagaaacaatgggtttcttcttgttttctcaaatgccttcattctttcttgtttcaactttgcttctttttcttattatttctcattcatctcatgctggaattttctcttcaaggaagtgcaagactccatctaagactttcaagggatattgtactagggattctaactgcgatacatcatgcagatacgagggctatcctgctggcgattaataatctaga

SEQ ID NO: 11 - DNA, 268 bp, Artificial Sequence
  Description of Artificial Sequence Synthetic chimeric nucleic acid encoding a signal peptide and SoD2 codon-optimized with CODA
tctagaaacaatgggtttctttttgttttctcaaatgccttcatttttccttgtgtctactcttcttctttttcttattatttctcattcttctcatgctggtatcttttctagtagaaagtgtaagactccttctaagacttttaaaggtatttgcactagagattctaattgtgacacttcttgtagatatgaaggttatcctgctggtgattgtaagggtattagaagaagatgtatgtgttctaagccttgttaataggagctc

SEQ ID NO: 12 - DNA, 226 bp, Artificial Sequence
  Description of Artificial Sequence Synthetic chimeric nucleic acid encoding a signal peptide and SoD7 codon-optimized with CODA
tctagaaacaatgggattctttttgttttctcaaatgccttctttctttcttgtgtctactcttcttctttttcttattatttctcattcttctcatgctggtattttttcatctcgtaagtgtaagactccttctaagacttttaagggttattgcactagagattctaattgtgatacatcttgtagatatgaaggttatcctgctggtgattaataggagctc

SEQ ID NO: 13 - DNA, 1462 bp, Artificial Sequence
  Description of Artificial Sequence Synthetic SoD2 expression cassette comprising a chimeric nucleic acid encoding a signal peptide and SoD2
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcatctagaaacaatgggcttcttccttttctctcaaatgccttcatttttccttgtttctactcttcttctttttcttattatttctcattcttctcatgctggtattttctcatctaggaagtgcaaaactccttcaaagacttttaagggaatttgcactagggattctaattgcgatacttcttgcagatacgagggatatccagctggcgattgcaaaggaattaggaggagatgtatgtgttcaaagccatgttaataatctagaacgcgtgaattcgaggcctcggatccctcgaggagctcggtacccggggtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgc

SEQ ID NO: 14 - DNA, 1420 bp, Artificial Sequence
  Description of Artificial Sequence Synthetic SoD7 expression cassette comprising a chimeric nucleic acid encoding a signal peptide and SoD7
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcatctagaaacaatgggtttcttcttgttttctcaaatgccttcattctttcttgtttcaactttgcttctttttcttattatttctcattcatctcatgctggaattttctcttcaaggaagtgcaagactccatctaagactttcaagggatattgtactagggattctaactgcgatacatcatgcagatacgagggctatcctgctggcgattaataatctagaacgcgtgaattcgaggcctcggatccctcgaggagctcggtacccggggtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgc

SEQ ID NO: 15 - DNA, 1424 bp, Artificial Sequence
  Description of Artificial Sequence Synthetic Chimeric nucleic acid encoding a signal peptide and SoD2
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcatctagaaacaatgggtttctttttgttttctcaaatgccttcatttttccttgtgtctactcttcttctttttcttattatttctcattcttctcatgctggtatcttttctagtagaaagtgtaagactccttctaagacttttaaaggtatttgcactagagattctaattgtgacacttcttgtagatatgaaggttatcctgctggtgattgtaagggtattagaagaagatgtatgtgttctaagccttgttaataggagctcggtacccggggtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgc

SEQ ID NO: 16 - DNA, 1388 bp, Artificial Sequence
  Description of Artificial Sequence Synthetic chimeric nucleic acid encoding a signal peptide and SoD7
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagcatctagaaacaatgggattctttttgttttctcaaatgccttctttctttcttgtgtctactcttcttctttttcttattatttctcattcttctcatgctggtattttttcatctcgtaagtgtaagactccttctaagacttttaagggttattgcactagagattctaattgtgatacatcttgtagatatgaaggttatcctgctggtgattaataggagctcggtacccggggtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgagaga

SEQ ID NO: 17 - DNA, 780 bp, Cauliflower mosaic virus
  promoter (1)..(780): CaMV 35S promoter
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctc

SEQ ID NO: 18 - DNA, 143 bp, Tobacco etch virus
  enhancer (1)..(143): TEV 5′UTR translational enhancer
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 19 - DNA, 205 bp, Cauliflower mosaic virus
  terminator (1)..(205): CaMV 35S terminator
tctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgc

SEQ ID NO: 20 - DNA, 31 bp, Artificial Sequence
  Description of Artificial Sequence Synthetic Zn5 primer
ccaatgcattgatcttcaaatgggaatgaat

SEQ ID NO: 21 - DNA, 28 bp, Artificial Sequence
  Description of Artificial Sequence Synthetic Zn6 primer
aactgcagttctaagaccagtcaaacta

SEQ ID NO: 22 - DNA, 40 bp, Artificial Sequence
  Description of Artificial Sequence Synthetic Fcp primer
ggcctctagagttatggacgacgagacatagtaattgaag

SEQ ID NO: 23 - DNA, 34 bp, Artificial Sequence
  Description of Artificial Sequence Synthetic Rcp primer
gcgcgagctcgatgaaactccaccatcccgatag

SEQ ID NO: 24 - DNA, 20 bp, Artificial Sequence
  Description of Artificial Sequence Synthetic GUSF primer
gtagaaaccccaacccgtga

SEQ ID NO: 25 - DNA, 21 bp, Artificial Sequence
  Description of Artificial Sequence Synthetic GUSR primer
gcggattcaccacttgcaaag

SEQ ID NO: 26 - protein, 83 aa, Artificial Sequence
  Description of Artificial Sequence Synthetic variant of SoD2 comprising two additional N-terminal amino acids and a Gly33 deletion relative to spinach SoD2
MGFFLFSQMPSFFLVSTLLLFLIISHSSHALEGIFSSRKCKTPSKTFKGICTRDSNCDTSCRYEYPAGDCKGIRRRCMCSKPC

SEQ ID NO: 27 - DNA, 265 bp, Artificial Sequence
  Description of Artificial Sequence Synthetic variant of SoD2 comprising two additional N-terminal amino acids and a Gly33 deletion relative to spinach SoD2
ttaattaatgggattctttctcttttcacaaatgccctcattctttcttgtgtcgacacttctcttattcctaataatatctcactcttctcatgcgctcgagggaatattcagctcccgcaagtgtaagacgccttcaaagactttcaaagggatatgtacgagagactcaaactgtgacacctcatgtcgttacgaatatccggcaggagactgtaaaggaatacgtcgcagatgtatgtgtagcaagccttgttagaggcct

SEQ ID NO: 28 - protein, 38 aa, Artificial Sequence
  Description of Artificial Sequence Synthetic core defensin based, in part, on Sod2 and Sod7
GIFSSRKCKTPSKTFKGXCTRDSNCDTSCRYEGYPAGD

SEQ ID NO: 29 - DNA, 120 bp, Artificial Sequence
  Description of Artificial Sequence Synthetic core defensin based, in part, on Sod2 and Sod7
ggaattttctcttcaaggaagtgcaagactccatctaagactttcaagggannntgtactagggattctaactgcgatacatcatgcagatacgagggctatcctgctggcgattaataa

SEQ ID NO: 30 - DNA, 174 bp, Artificial Sequence
  Description of Artificial Sequence Synthetic SoD2 codon-optimized with DNA 2.0
tctagaatgggaatcttcagttcgagaaagtgtaaaaccccctcaaaaacattcaaaggtatttgcacgagagattctaattgcgatactagctgccgttatgagggttaccctgctggcgactgtaaggggataaggaggagatgtatgtgctccaagccatgttaaggtacc

SEQ ID NO: 31 - DNA, 132 bp, Artificial Sequence
  Description of Artificial Sequence Synthetic SoD7 codon-optimized with DNA 2.0
tctagaatgggtatcttctcaagcagaaagtgcaaaacaccttctaaaacctttaagggatattgtactagggactccaattgtgatacgagttgccgttacgagggctatccagctggggattaaggtacc

SEQ ID NO: 32 - DNA, 27 bp, Tobacco etch virus
atgaaaacttacaatgttggagggatg

SEQ ID NO: 33 - protein, 9 aa, Tobacco etch virus
MKTYNVGGM

SEQ ID NO: 34 - DNA, 27 bp, Artificial Sequence
  Description of Artificial Sequence Synthetic oligonucleotide
atgaagacctataacgtaggtggcatg

SEQ ID NO: 35 - DNA, 21 bp, Tobacco etch virus
gagaatctttattttcagagt

SEQ ID NO: 36 - protein, 7 aa, Tobacco etch virus
ENLYFQS

SEQ ID NO: 37 - DNA, 21 bp, Artificial Sequence
  Description of Artificial Sequence Synthetic oligonucleotide
gaaaacctatacttccaatcg

SEQ ID NO: 38 - DNA, 33 bp, Artificial Sequence
  Description of Artificial Sequence Synthetic primer
agtcctcgagaaccacttagttgtttagctatc

SEQ ID NO: 39 - DNA, 42 bp, Artificial Sequence
  Description of Artificial Sequence Synthetic primer
ttatgcggccgcaggccttggacctatgttggccccccatag

SEQ ID NO: 40 - DNA, 42 bp, Artificial Sequence
  Description of Artificial Sequence Synthetic primer
taatcgtacttgagttctaatatggctagcaaaggagaagaa

SEQ ID NO: 41 - DNA, 100 bp, Artificial Sequence
  Description of Artificial Sequence Synthetic primer
gccgcactagtatttaaatcccgtttcgtcctttagggactcgtcagtgtactgatataagtacagactggacctatgttggccccccatagggacagtg

SEQ ID NO: 42 - DNA, 44 bp, Artificial Sequence
  Description of Artificial Sequence Synthetic primer
atggatgagctctacaaatgattgaagtggacggaataagttcc

SEQ ID NO: 43 - DNA, 44 bp, Artificial Sequence
  Description of Artificial Sequence Synthetic primer
ggaacttattccgtccacttcaatcatttgtagagctcatccat

SEQ ID NO: 44 - DNA, 84 bp, Artificial Sequence
  Description of Artificial Sequence Synthetic primer
gcacgttgtgctatagtacgtgccataatagtgagtgctagcaaagtataaacgctggtgtttagcgcatattaaatactaacg

SEQ ID NO: 45 - DNA, 101 bp, Artificial Sequence
  Description of Artificial Sequence Synthetic primer
cagcttgcttctacctgacacagttaagaagcggcataaatcgaagccaaaccctaaattttgcaactcgatcaattgtaacctagagcgaagtgcaatca

SEQ ID NO: 46 - DNA, 45 bp, Artificial Sequence
  Description of Artificial Sequence Synthetic primer
tttagcgcatattaaatactaacgatggctagcaaaggagaagaa

SEQ ID NO: 47 - DNA, 45 bp, Artificial Sequence
  Description of Artificial Sequence Synthetic primer
actgtgtcaggtagaagcaagctgtcagatgaagtggtgttcacg

SEQ ID NO: 48 - DNA, 45 bp, Artificial Sequence
  Description of Artificial Sequence Synthetic primer
ttggatttaggtgacactatagtggacctatgttggccccccata

SEQ ID NO: 49 - DNA, 45 bp, Artificial Sequence
  Description of Artificial Sequence Synthetic primer
gtaacctagagcgaagtgcaatcaatggctagcaaaggagaagaa

SEQ ID NO: 50 - DNA, 86 bp, Artificial Sequence
  Description of Artificial Sequence Synthetic primer
gcctaagcttacaaatactcccccacaacagcttacaatactcccccacacagcttacaaatactcccccacaacagcttgtcgac

SEQ ID NO: 51 - DNA, 45 bp, Artificial Sequence
  Description of Artificial Sequence Synthetic primer
ctccgtgaacaccacttcatctgaaaataacaaatctcaacacaa

SEQ ID NO: 52 - DNA, 45 bp, Artificial Sequence
  Description of Artificial Sequence Synthetic primer
ttgtgttgagatttgttattttcagatgaagtggtgttcacggag

SEQ ID NO: 53 - DNA, 45 bp, Artificial Sequence
  Description of Artificial Sequence Synthetic primer
ggagtatttgtaagcttaggctcagatgaagtggtgttcacggag

SEQ ID NO: 54 - DNA, 45 bp, Artificial Sequence
  Description of Artificial Sequence Synthetic primer
ccccacaacagcttgtcgacatggctagcaaaggagaagaacttt

SEQ ID NO: 55 - DNA, 45 bp, Artificial Sequence
  Description of Artificial Sequence Synthetic primer
cgtgaacaccacttcatctgattcgacctcggtcgtcttagttaa

SEQ ID NO: 56 - DNA, 45 bp, Artificial Sequence
  Description of Artificial Sequence Synthetic primer
ttaactaagacgaccgaggtcgaatcagatgaagtggtgttcacg

SEQ ID NO: 57 - DNA, 137 bp, Artificial Sequence
  Description of Artificial Sequence Synthetic primer
ggcgatcacgacagagccgtgtcaattgtcgcggctaagaatgctgtggatcgcagcgctttcactggaggggagagaaaaatagttagtttgtatgccttaggaaggaactaagcacgttgtgctatagtacgtgc

SEQ ID NO: 58 - DNA, 45 bp, Artificial Sequence
  Description of Artificial Sequence Synthetic primer
tgacacggctctgtcgtgatcgcctcagatgaagtggtgttcacg

SEQ ID NO: 59 - DNA, 45 bp, Artificial Sequence
  Description of Artificial Sequence Synthetic primer
gccacctacgttataggtcttcattttgtagagctcatccatgcc

SEQ ID NO: 60 - DNA, 45 bp, Artificial Sequence
  Description of Artificial Sequence Synthetic primer
aagacctataacgtaggtggcatgaaggctcaatattcggatcta

SEQ ID NO: 61 - DNA, 45 bp, Artificial Sequence
  Description of Artificial Sequence Synthetic primer
atgaaaacttacaatgttggagggatgttacgtcctgtagaaacc

SEQ ID NO: 62 - DNA, 45 bp, Artificial Sequence
  Description of Artificial Sequence Synthetic primer
ggtttctacaggacgtaacatccctccaacattgtaagttttcat

SEQ ID NO: 63 - DNA, 45 bp, Artificial Sequence
  Description of Artificial Sequence Synthetic primer
ccgcagcagggaggcaaacaatgattgaagtggacggaataagtt

SEQ ID NO: 64 - DNA, 45 bp, Artificial Sequence
  Description of Artificial Sequence Synthetic primer
aacttattccgtccacttcaatcattgtttgcctccctgctgcgg

SEQ ID NO: 65 - DNA, 45 bp, Artificial Sequence
  Description of Artificial Sequence Synthetic primer
cttactctgaaaataaagattctctttgtagagctcatccatgcc

SEQ ID NO: 66 - DNA, 45 bp, Artificial Sequence
  Description of Artificial Sequence Synthetic primer
aaagagaatctttattttcagagtaagggaccacgtgattacaac

SEQ ID NO: 67 - DNA, 45 bp, Artificial Sequence
  Description of Artificial Sequence Synthetic primer
cgattggaagtataggttttcttgcgagtacaccaattcactcat

SEQ ID NO: 68 - DNA, 45 bp, Artificial Sequence
  Description of Artificial Sequence Synthetic primer
caagaaaacctatacttccaatcgatgttacgtcctgtagaaacc

SEQ ID NO: 69 - DNA, 45 bp, Artificial Sequence
  Description of Artificial Sequence Synthetic primer
gtcactttgtttagcgtgacttagcagcttgcttctacctgacac

SEQ ID NO: 70 - DNA, 45 bp, Artificial Sequence
  Description of Artificial Sequence Synthetic primer
gtgtcaggtagaagcaagctgctaagtcacgctaaacaaagtgac

SEQ ID NO: 71 - DNA, 45 bp, Artificial Sequence
  Description of Artificial Sequence Synthetic primer
ttagtctctccatcttgcgtgtagcagcttgcttctacctgacac

SEQ ID NO: 72 - DNA, 45 bp, Artificial Sequence
  Description of Artificial Sequence Synthetic primer
gtgtcaggtagaagcaagctgctacacgcaagatggagagactaa

SEQ ID NO: 73 - DNA, 45 bp, Artificial Sequence
  Description of Artificial Sequence Synthetic primer
atggatgagctctacaaatgagtttcagaaattgtcgaatcgcat

SEQ ID NO: 74 - DNA, 45 bp, Artificial Sequence
  Description of Artificial Sequence Synthetic primer
atgcgattcgacaatttctgaaactcatttgtagagctcatccat

SEQ ID NO: 75 - DNA, 45 bp, Artificial Sequence
  Description of Artificial Sequence Synthetic primer
atggatgagctctacaaatgagttaatacgcttctcagaacgtgt

SEQ ID NO: 76 - DNA, 45 bp, Artificial Sequence
  Description of Artificial Sequence Synthetic primer
acacgttctgagaagcgtattaactcatttgtagagctcatccat

SEQ ID NO: 77 - DNA, 45 bp, Artificial Sequence
  Description of Artificial Sequence Synthetic primer
tttagcgcatattaaatactaacgatgtacccatacgatgttcca

SEQ ID NO: 78 - DNA, 45 bp, Artificial Sequence
  Description of Artificial Sequence Synthetic primer
tggaacatcgtatgggtacatcgttagtatttaatatgcgctaaa

SEQ ID NO: 79 - DNA, 45 bp, Artificial Sequence
  Description of Artificial Sequence Synthetic primer
actgtgtcaggtagaagcaagctgttacttgtacagctcgtccat

SEQ ID NO: 80 - DNA, 45 bp, Artificial Sequence
  Description of Artificial Sequence Synthetic primer
gtaacctagagcgaagtgcaatcaatggactacaaagacgatgac

SEQ ID NO: 81 - DNA, 45 bp, Artificial Sequence
  Description of Artificial Sequence Synthetic primer
gtcactttgtttagcgtgacttagggcgatcacgacagagccgtg

SEQ ID NO: 82 - DNA, 45 bp, Artificial Sequence
  Description of Artificial Sequence Synthetic primer
cacggctctgtcgtgatcgccctaagtcacgctaaacaaagtgac

SEQ ID NO: 83 - DNA, 45 bp, Artificial Sequence
  Description of Artificial Sequence Synthetic primer
gtcactttgtttagcgtgacttagttcgacctcggtcgtcttagt

SEQ ID NO: 84 - DNA, 45 bp, Artificial Sequence
  Description of Artificial Sequence Synthetic primer
actaagacgaccgaggtcgaactaagtcacgctaaacaaagtgac

SEQ ID NO: 85 - DNA, 45 bp, Artificial Sequence
  Description of Artificial Sequence Synthetic primer
cacaacgtctatatcatggcctaggtttcagaaattgtcgaatcg

SEQ ID NO: 86 - DNA, 45 bp, Artificial Sequence
  Description of Artificial Sequence Synthetic primer
cgattcgacaatttctgaaacctaggccatgatatagacgttgtg

SEQ ID NO: 87 - DNA, 45 bp, Artificial Sequence
  Description of Artificial Sequence Synthetic primer
ggcatggacgagctgtacaagtaattgaagtggacggaataagtt

SEQ ID NO: 88 - DNA, 45 bp, Artificial Sequence
  Description of Artificial Sequence Synthetic primer
aacttattccgtccacttcaattacttgtacagctcgtccatgcc

SEQ ID NO: 89 - DNA, 45 bp, Artificial Sequence
  Description of Artificial Sequence Synthetic primer
tcgctcttaccttgcgataactagcagcttgcttctacctgacac

SEQ ID NO: 90 - DNA, 45 bp, Artificial Sequence
  Description of Artificial Sequence Synthetic primer
gtaacctagagcgaagtgcaatcaatgttacgtcctgtagaaacc

SEQ ID NO: 91 - DNA, 47 bp, Artificial Sequence
  Description of Artificial Sequence Synthetic primer
ggtttctacaggacgtaacattgattgcacttcgctctaggttacaa

SEQ ID NO: 92 - DNA, 45 bp, Artificial Sequence
  Description of Artificial Sequence Synthetic primer
ccgcagcagggaggcaaacaatgagtttcagaaattgtcgaatcg

SEQ ID NO: 93 - DNA, 45 bp, Artificial Sequence
  Description of Artificial Sequence Synthetic primer
cgattcgacaatttctgaaactcattgtttgcctccctgctgcgg

SEQ ID NO: 94 - DNA, 45 bp, Artificial Sequence
  Description of Artificial Sequence Synthetic primer
gtgtcaggtagaagcaagctgctagttatcgcaaggtaagagcga

SEQ ID NO: 95 - DNA, 45 bp, Artificial Sequence
  Description of Artificial Sequence Synthetic primer
atggatgagctctacaaatgaagtctactcagtagtacgtctatt

SEQ ID NO: 96 - DNA, 45 bp, Artificial Sequence
  Description of Artificial Sequence Synthetic primer
aatagacgtactactgagtagacttcatttgtagagctcatccat

SEQ ID NO: 97 - DNA, 45 bp, Artificial Sequence
  Description of Artificial Sequence Synthetic primer
gcggatgcattatttggttttacaacaacggtacgtttcaaaatg

SEQ ID NO: 98 - DNA, 45 bp, Artificial Sequence
  Description of Artificial Sequence Synthetic primer
atgaaaacttacaatgttggagggatggctagcaaaggagaagaa

SEQ ID NO: 99 - DNA, 45 bp, Artificial Sequence
  Description of Artificial Sequence Synthetic primer
ttcttctcctttgctagccatccctccaacattgtaagttttcat

SEQ ID NO: 100 - DNA, 43 bp, Artificial Sequence
  Description of Artificial Sequence Synthetic primer
gagaatctttattttcagagtaagggaccacgtgattacaacc

SEQ ID NO: 101 - DNA, 44 bp, Artificial Sequence
  Description of Artificial Sequence Synthetic primer
gaaaacctatacttccaatcgatggctagcaaaggagaagaact

SEQ ID NO: 102 - DNA, 44 bp, Artificial Sequence
  Description of Artificial Sequence Synthetic primer
agttcttctcctttgctagccatcgattggaagtataggttttc

SEQ ID NO: 103 - DNA, 45 bp, Artificial Sequence
  Description of Artificial Sequence Synthetic primer
aagacctataacgtaggtggcatgaagggaccacgtgattacaac

SEQ ID NO: 104 - DNA, 45 bp, Artificial Sequence
  Description of Artificial Sequence Synthetic primer
ccctccaacattgtaagttttcatttgcgagtacaccaattcact

SEQ ID NO: 105 - DNA, 45 bp, Artificial Sequence
  Description of Artificial Sequence Synthetic primer
gagaatctttattttcagagtaaggctcaatattcggatctaaag

SEQ ID NO: 106 - DNA, 45 bp, Artificial Sequence
  Description of Artificial Sequence Synthetic primer
cgattggaagtataggttttcttcggattccaaacctgaatgaac

SEQ ID NO: 107 - DNA, 45 bp, Artificial Sequence
  Description of Artificial Sequence Synthetic primer
gccacctacgttataggtcttcatgatgaagtggtgttcacggag

SEQ ID NO: 108 - DNA, 45 bp, Artificial Sequence
  Description of Artificial Sequence Synthetic primer
actctgaaaataaagattctcgatgaagtggtgttcacggagaac

SEQ ID NO: 109 - DNA, 39 bp, Artificial Sequence
  Description of Artificial Sequence Synthetic primer
catttacgaacgatagccatggctagcaaaggagaagaa

SEQ ID NO: 110 - protein, 51 aa, Artificial Sequence
  Description of Artificial Sequence Synthetic Putative mature SoD2 peptide with a Gly33 deletion relative to spinach SoD2
GIFSSRKCKTPSKTFKGICTRDSNCDTSCRYEYPAGDCKGIRRRCMCSKPC

(19) United States
(12) Patent Application Publication
(10) Pub. No.: US 2015/0067918 A1
(43) Pub. Date: Mar. 5, 2015

(54) CITRUS PLANTS RESISTANT TO CITRUS HUANGLONGBING (EX GREENING) CAUSED BY CANDIDATUS LIBERIBACTER ASIATICUS (LAS) AND BACTERIAL CANKER CAUSED BY (XANTHOMONAS AXONOPODIS PV. CITRI) (XAC)

(71) Applicant: Southern Gardens Citrus, Clewiston, FL (US)

(72) Inventor: Ricke Kress, Clewiston, FL (US)

(73) Assignee: SOUTHERN GARDENS CITRUS, Clewiston, FL (US)

(21) Appl. No.: 14/468,998

(22) Filed: Aug. 26, 2014

Related U.S. Application Data
(60) Provisional application No. 61/871,089, filed on Aug. 28, 2013.

(51) Int. Cl. C12N 15/82; C12N 7/00; C07K 14/415

(52) U.S. Cl. CPC C12N 15/8281; C07K 14/415; C12N 7/00; C12N 2770/24043
USPC 800/280; 435/320.1; 435/419; 800/301; 800/279

(57) ABSTRACT

Disclosed herein are viral vectors based on modifications of the Citrus Tristeza virus useful for transfecting citrus trees for beneficial purposes. Included in the disclosure are viral vectors including one or more gene cassettes that encode spinach defensin peptide(s). The gene cassettes are positioned at desirable locations on the viral genome so as to enable expression while preserving functionality of the virus. Also disclosed are methods of transfecting plants and plants transfected with viral vector embodiments.

### CROSS REFERENCE TO RELATED APPLICATIONS

The present application relates to and claims priority from U.S. provisional patent application No. 61/871,089, entitled CITRUS PLANTS RESISTANT TO CITRUS HUANGLONGBING (EX GREENING) CAUSED BY CANDIDATUS LIBERIBACTER ASIATICUS (LAS) AND BACTERIAL CANKER CAUSED BY (XANTHOMONAS AXONOPODIS PV. CITRI) (XAC), filed on Aug. 28, 2013. The disclosure of the above-identified co-pending provisional application is hereby incorporated by reference in their entirety.

### BACKGROUND

Citrus greening, also called Huanglongbing or yellow dragon disease, is one of the more serious diseases of citrus. This bacterial disease is thought to have originated in China in the early 1900s. The disease is primarily spread by two species of psyllid insects. One species, the Asian citrus pysllid, Diaphorina citri, has been present in Florida since 1998. The bacteria itself is not harmful to humans but the disease has harmed trees in Asia, Africa, the Arabian Peninsula, and Brazil. There are three strains of the bacteria, an Asian, an African version, and a recently described American strain discovered in Brazil.

The Asian strain, Candidatus Liberibacter asiaticus was found in Florida in early September, 2005. To respond to the problem, USDA, APHIS, PPQ and the Florida Department of Agriculture and Consumer Services deployed a Unified Command under the Incident Command Structure, and delimiting survey crews are working in southern Florida to define the extent of the problem.

Citrus greening disease is a threat to the U.S. citrus industry. Other than tree removal, there is no effective control once a tree is infected and there is no known cure for the disease. Infected trees may produce misshapen, unmarketable, bitter fruit. Citrus greening reduces the quantity and quality of citrus fruits, eventually rendering infected trees useless. In areas of world affected by citrus greening the average productive lifespan of citrus trees has dropped from 50 or more years to 15 or less. The trees in the orchards usually die 3-5 years after becoming infected and require removal and replanting. An infected tree produces fruit that is unsuitable for sale as fresh fruit or for juice.

Citrus plants infected by the citrus greening bacteria may not show symptoms for years following infection. Initial symptoms frequently include the appearance of yellow shoots on a tree. As the bacteria move within the tree, the entire canopy progressively develops a yellow color. The most characteristic symptoms of citrus greening are a blotchy leaf mottle and vein yellowing that develop on leaves attached to shoots showing the overall yellow appearance. These foliar symptoms may superficially resemble a zinc deficiency although the green and yellow contrast is not as vivid with greening as it is with zinc deficiency or another disease, citrus variegated chlorosis. Leaves with citrus greening have a mottled appearance that differs from nutrition-related mottling in that greening-induced mottling usually crosses leaf veins. Nutrition related mottles usually are found between or along leaf veins and leaves may be small and upright.

Fruit from diseased trees are small, often misshapen, and typically some green color remains on ripened fruit. On Mandarin orange, fruit may develop an uneven ripening such that they appear half orange and half yellow. This symptom is the origin of the common name “greening.” Yields are almost non-existent, and remaining fruit is rendered worthless due to small size, poor color, and bad taste.

The only definitive method of diagnosis of trees suspected of infection by citrus greening pathogens is by analysis of DNA in an authorized plant diagnostic laboratory.

Citrus greening has been reported from the following countries in Africa, Asia and South America: Bangladesh, Belize, Bhutan, Brazil, Burundi, Cambodia, Cameroon, Central African Republic, China, Comoros, Cuba, Dominican Republic, Ethiopia, Guatemala, Honduras, Hong Kong, India, Indonesia, Iran, Jamaica, Japan, Kenya, Laos, Madagascar, Malawi, Malaysia, Mauritius, Mexico, Myanmar, Nepal, Pakistan, Papua New Guinea, Philippines, Reunion, Rwanda, Saudi Arabia, Somalia, South Africa, Sri Lanka, Swaziland, Taiwan, Tanzania, Thailand, Vietnam, Yemen, and Zimbabwe.

There is currently no known preventment or treatment of infected trees. Growers have tried various nutritional programs and foliar sprays but to no avail. At present, there are no Citrus cultivars resistant to bacterial canker (Xanthomonas axonopodis pv. citri) (Xac), and/or citrus Huanglongbing (ex greening) caused by Candidatus Liberibacter asiaticus (Las). Indeed, no genetic resistance to these microbial pathogens has ever been found within the Citrus genus. Conventional cross-breeding efforts to produce resistant cultivars have been hindered by the complex reproductive biology and long life cycle of Citrus spp.

The present invention meets this need as it provides a suitable viral vector that can provide long term expression of a spinach defensin gene(s) in citrus trees which when expressed provides the citrus plant resistance to bacterial canker (Xanthomonas axonopodis pv. citri) (Xac), and/or citrus Huanglongbing (ex greening) caused by Candidatus Liberibacter asiaticus (Las).

### SUMMARY

The present invention is based on the use of a citrus tristeza virus (“CTV”) vector to express codon enhanced spinach defensin genes as well as native spinach defensin genes in a citrus plant that can produce enough gene product to be useful in imparting resistance to bacterial canker (Xanthomonas axonopodis pv. citri) (Xac), and/or citrus Huanglongbing (ex greening) caused by Candidatus Liberibacter asiaticus (Las). Vector constructs of the present invention can continue to replicate and spread effectively in the plant; and remain stable in the plant long enough to be useful. In one embodiment, gene cassettes are introduced into the CTV genome as replacement of the p13 gene. In other embodiments, a spinach defensin gene(s) is inserted at different locations (e.g., p13-p20, p20-p23 and p23-3′NTR (non-translated region)). In another embodiment, a fusion to p23 and protease processing can be used. In alternative embodiments, a spinach defensin gene(s) is inserted behind IRES sequences to create bi-cistronic messages.

The genetic constructs of the invention preferably spread systemically in plants, and produce the spinach defensin peptide(s). Examples of the expression vectors include the “add a gene” constructs having an insertion of the spinach defensin gene between the p13 and p20 genes or between the p23 gene and the 3′NTR. Similarly, the present invention provides CTV vectors with the spinach defensin gene(s) replacing the p13 gene, or after the p13 gene, or after the p23 gene or between the minor coat protein (CPm) and the coat protein (CP).

The novel CTV constructs disclosed herein have genomes with unique elasticity capable of accommodating and expressing more than one foreign gene/s by and preferably more than one spinach defensin gene.

Engineering an effective vector requires a balance between different factors. The vector needs to be designed such that replication and systemic movement in the plant are reduced minimally while the level of expression of the foreign protein is maximal (Shivprasad et al., 1999). The final factor is the stability of the vector. In general, the vector's usefulness is directly correlated with its stability. Stability is a product of reduced recombination and increased competitiveness of the vector with the resulting recombinants that have lost part or all of the inserted sequences.

### BRIEF DESCRIPTION OF DRAWINGS

CTV Vector Figures:

FIG. 1. GFP replacement of p13 to produce CTV based expression vectors. (A) Schematic representation of CTV9RΔp33(Boxes represent open reading frames with blue outline of boxes represent the replication gene block whereas the red outline represent the closterovirus conserved gene block (Karasev, 2000). The black circle and black boxes outline represent silencing suppressors (Lu et al., 2004). Gold box outline represent genes dispensible for the infection of some citrus genotypes (Tatineni et al., 2008). Filled black rectangle represents the deletion of the p33 controller elements and ORF (nts 10858-11660 Genebank Accession #AY170468) (Satyanarayana et al., 1999; 2000; 2003)). Arrows indicate the processing of the leader proteases of CTV, LP1 and LP2 are two tandem leader protease, MT (methyl transferase), Hel (Helicase), RdRp (RNA dependent RNA polymerase, Δp33(deletion of the 33kda protein sequence), p6 (6kda protein), Hsp7Oh (heat shock protein 70 homologue), p61 (61kda protein), CPm (minor coat protein), CP (major coat protein, inter cellular silencing suppressor), p18 (18 kda protein), p13 (13 kda protein), p20 (20 kda protein, inter/intra cellular silencing suppressor), p23 (23 kda protein, intracellular silencing suppressor) and modification to produce expression vectors CTV33-Δ13-BY-GFP-57 (C57), CTV33-Δ13-G-GFP-65 (C65), CTV33-Δ13-B-GFP-66 (C66) with the CP-CE of BYSV, GLRaV-2 and BYV driving GFP, respectively. (B) Northern blot analysis of wild type CTV (WT) and CTV based expression vector transfected to N. benthamiana protoplast (T) and passaged to a new set of protoplasts (P). (C) Representative sample of fluorescence in N. benthamiana infected with either of the three constructs CTV33-Δ13-BY-GFP-57, CTV33-Δ13-G-GFP-65, CTV33-Δ13-B-GFP-66 magnified under a fluorescent stereoscope. (D) Representative sample of fluorescence in the phloem of citrus bark pieces infected with constructs CTV33-Δ13-G-GFP-65 and CTV33-Δ13-B-GFP-66 with high (left) and low (right) magnification under a fluorescent stereoscope.

FIG. 2 GUS replacement of p13 to produce CTV based expression vectors. (A) Schematic representation of CTV9RΔp33and its modification creating expression vector CTV33-Δ13-BY-GUS-61 in which the p13 and its controller element is replaced by GUS under the control of CP-CE of BYSV. (B) Northern blot hybridization analysis of wild type CTV (WT) and CTV based expression vector CTV33-Δ13-BY-GUS-61 (C61) transfected to N. benthamiana protoplast (T) and passaged to a new set of protoplasts (P). (C) Representative sample of GUS activity in the bark pieces of citrus trees infected with construct CTV33-Δ13-BY-GUS-61(right) and the GUS solution before fixing of the bark pieces (left) (A=Healthy control, B=infect).

FIG. 3 GFP insertion between p13 and p20 to produce CTV based expression vectors. (A) Schematic representation of CTV9RΔp33and modification by inserting between p13 and p20 of GFP ORF under the control of BYSV creating expression vector CTV33-13-BY-GFP-69 (B) Northern blot hybridization analysis of transfected protoplast with the wild type virus (WT) and expression vector CTV33-13-BY-GFP-69 (C69) from transcripts (T) and their passages (P). Representative sample of fluorescence in N. benthamiana (C) and peeled bark phloem pieces of C. macrophylla (D) infected with CTV33-13-BY-GFP-69 magnified under a fluorescent stereoscope.

FIG. 4 GFP insertion between p20 and p23 to produce CTV based expression vectors. (A) Schematic representation of CTV9RΔp33and its modification producing expression vector CTV33-20-B-GFP-49 and CTV33-20-BY-GFP-58, respectively. (B) Northern blot hybridization analysis of transfected protoplast with the wild type virus (WT) and expression vectors CTV33-20-B-GFP-49 (C49) and CTV33-20-BY-GFP-58 (C58) from transcripts (T) and their passages (P). (C) Flourescence under UV light of protoplast (right) and the leaf (left) showing lack of efficient movement of the vector. (D) Western blot analysis of the same gene inserted at different locations in the CTV genome. BCN5 (Folimonov et al., 2007) original CTV vector (contains GFP under BYV promoter between CPm and CP), constructs CTV33-23-BY-GFP-37 (C37, insertion of BYSV driving GFP behind p23), CTV33-20-BY-GFP-58 (C58, insertion of BYSV driving GFP between p20 and p23), CTV33-13-BY-GFP-69 (C69, insertion of BYSV driving GFP between p13 and p20), CTV33-Δ13-BY-GFP-57(C57, replacement of p13 gene with BYSV CP-CE driving GFP) and CTV33-27-BY-GFP-63 (C63, Insertion of BYSV CP-CE driving GFP ORF between CPm and CP).

FIG. 5 GFP insertion between p23 and 3′NTR to produce CTV based expression vectors. (A) Schematic representation of CTV9RΔp33and its modification by insertion of GFP behind p23 under control of CP-CE of BYSV, GLRaV-2 and BYV creating expression CTV33-23-BY-GFP-37 (C37), CTV33-23-G-GFP-40 (C40) and CTV33-23-B-GFP-42 (C42), respectively. (B) Northern blot hybridization analysis of transfected protoplast with the wild type virus (WT) and expression vectors CTV33-23-BY-GFP-37, CTV33-23-G-GFP-40 and CTV33-23-B-GFP-42 from transcripts (T) and their passages (P). (C) Representative sample of fluorescence in N. benthamiana infected with either of the three constructs CTV33-23-BY-GFP-37, CTV33-23-G-GFP-40 and CTV33-23-B-GFP-42 magnified under a fluorescent stereoscope. (D) Representative sample of fluorescence in the phloem tissue of Citrus macropylla infected with constructs CTV33-23-BY-GFP-37 and CTV33-23-G-GFP-40.

FIG. 6 GUS insertion between p23 and 3′NTR insertion between p23 and 3′NTR to produce CTV based expression vectors. (A) Schematic representation of CTV9RΔp33and modification by insertion of GUS ORF under control of BYSV CP-CE between p23 and 3′NTR creating expression vector CTV33-23-BY-GUS-60 (C60). (B) Northern blot hybridization analysis of transfected protoplast with the wild type virus (WT) and expression vectors CTV33-23-BY-GUS-60 from transcripts (T). (C) Enzymatic activity of the GUS protein in N. benthamiana tissue and citrus phloem bark pieces (Blue color indicate infected plant and colorless tissue and solution indicate healthy control and GUS solution subject to the same treatment.

FIG. 7 GFP inserted behind IRES sequences to create CTV based expression vectors. (A) Schematic representation of CTV9RΔp33and CTVΔCla 333R and their modification behind p23 creating expression vectors CTV33-23-ITEV-GFP-41;CTV33-23-I3XARC-GFP-43 represent the TEV 5′NTR IRES and 3xARC-1 IRES, respectively and CTVp333R-23-ITEV-GFP; CTVp333R-23-I3XARC-GFP representing the TEV 5′NTR IRES and 3xARC-1 IRES, respectively. (B) 1-Northern blot hybridization analysis from tranfected N. benthamiana protoplast with wild type virus (WT), CTV33-23-ITEV-GFP-41 (C41) and CTV33-23-I3XARC-GFP-43 (C43); T=RNA isolated from transcript transfected protoplast and P=RNA isolated from virion transfected protoplast isolated from RNA transfected protoplast. 2-Northern blot hybridization analysis from protoplast transfected with CTVp333R-23-ITEV-GFP (Lane A); CTVp333R-23-I3XARC-GFP (lane B), CTVp333R (lane C) and CTVp333R-23-B-GFP (BYV CP-CE driving the expression of GFP behind p23) (Lane D).

FIG. 8 GFP and a protease fused to p23 to create CTV based expression vectors. (A) Schematic representation of CTV9RΔp33and the modifications by fusing two TEV proteases (NIa and HC-Pro) and their recognition sequences to create expression vectors CTV33-23-HC-GFP-72, CTV33-23-NIa-GFP-73, CTV33-23-HCØ-GFP-74 and CTV33-23-NIaØ-GFP-75.

FIG. 9 Comparison of Florescence in N. benthamiana. (A) Comparison of fluorescence in infiltrated leaves of representative samples of constructs CTV33-23-HC-GFP-72, CTV33-23-NIa-GFP-73, CTV33-23-HCØ-GFP-74 and CTV33-23-NIaØ-GFP-75 (GFP fused) and CTV33-23-BY-GFP-37, CTV33-23-G-GFP-40 and CTV33-23-B-GFP-42 (free GFP) under hand held UV light (Right) and the same leaves under white light (left). (B) Comparison on whole plant level between representative samples of constructs CTV33-23-HC-GFP-72 and CTV33-23-NIa-GFP-73 (fused GFP) and CTV33-23-BY-GFP-37, CTV33-23-G-GFP-40 and CTV33-23-B-GFP-42 (GFP under its own controller element behind p23 (Free GFP)) under hand held UV light (Right) and same plants under white light (Left). (C) Comparison between the abaxial (Lower) and adaxial (upper) leaf surfaces of the same representative leaf sample of constructs CTV33-23-HC-GFP-72 and CTV33-23-NIa-GFP-73 under hand held UV light (Right) and white light (Left).

FIG. 10 Western blot analysis of different expression vectors infiltrated into N. benthamiana leaves using GFP antibody. A=CTV9RΔp33GFP (GFP inserted under the BYV CP-CE controller element between CPm and CP (produces free GFP) (Tatineni et al., 2008)), B=CTV33-23-BY-GFP-HC-GUS-51, C=CTV33-23-G-GFP-NIa-GUS-54, D=Empty well; E=CTV33-Δ13-BY-GFP-NIa-GUS-78, F=CTV33-23-HC-GFP-72, G=CTV33-23-NIa-GFP-73.

FIG. 11 Hybrid gene (GFP/Protease/GUS fusion) replacement of p13 to create expression vectors. (A) Schematic representation of CTV9R Δp33 and its modification to create expression vectors CTV33-Δ13-BYGFP-HC-GUS-77 and CTV33-Δ13-BYGFP-NIa-GUS-78 with the two fusion genes under the control of BYSV CP-CE with TEV HC-Pro and NIa spanned by their proteolysis recognition sequence separating GFP and GUS, respectively. (B) Activity of the reporter genes in N. benthamiana and Citrus macrophylla. (a.) Representative sample of N. benthamiana plant infected with either CTV33-Δ13-BYGFP-HC-GUS-77 or CTV33-Δ13-BYGFP-NIa-GUS-78 N. benthamiana under white light and (b.) the same plant under UV light (c.) Two pictures of peeled phloem bark pieces of C. macrophylla infected with construct CTV33-Δ13-BYGFP-NIa-GUS-78 under a fluorescent stereoscope (d.) Representative sample of GUS activity in systemic N. benthamiana leaves, control leaf (Left) and infected leaf (right) (e.) Peeled bark phloem pieces and GUS solution of healthy C. macrophylla plant (f.) Peeled bark phloem pieces of C. macrophylla plant infected with construct CTV33-Δ13-BYGFP-NIa-GUS-78.

FIG. 12 Stability of Constructs in N. benthamiana. (A) Upper leaf from Agro-inoculated N. benthamiana plants carrying the binary vector CTV33-Δ13-BYGFP-HC-GUS-77 (GFP/HC-Pro/GUS) pictured under fluorescent microscope. (B) The same leaf was tested for GUS activity indicating almost perfect overlap between the two reporter genes.

FIG. 13 Hybrid gene (GFP/Protease/GUS fusion) between p23 and 3′NTR to create expression vectors. (A) Schematic representation of CTV9R Δp33 and its modification to produce expression vectors CTV33-23-BY-GFP-HC-GUS-51 and CTV33-23-BY-GFP-NIa-GUS-52 has the BYSV CP-CE driving the hybrid genes that contain HC-Pro and NIa proteases respectively; CTV33-23-G-GFP-HC-GUS-53 (C53) and CTV33-23-G-GFP-NIa-GUS-54 (C54) are GLRaV-2 driven fusion genes that contain the HC-Pro and NIa proteases, respectively; CTV33-23-BY-GFP-HC-GUS-55 (C55) and CTV33-23-BY-GFP-NIa-GUS-56 (C56) are BYV driven fusion genes that contain HC-Pro and NIa proteases, respectively. (B) Northern blot hybridization analysis of transfected protoplast with wild type virus (WT), C53, C54, C55 and C56 constructs.

FIG. 14 Activity of reporter genes generated by insertion of the Hybrid gene (GFP/Protease/GUS fusion) behind p23. (A) Activity of the reporter genes in N. benthamiana plants (a.) Representative sample of N. benthamiana plant infected with CTV33-23-BY-GFP-HC-GUS-51, CTV33-23-G-GFP-HC-GUS-53, CTV33-23-BY-GFP-NIa-GUS-52 or CTV33-23-G-GFP-NIa-GUS-54 under white light and (b.) the same plant under hand held UV light (c.) Representative sample of GUS activity in infected systemic N. benthamiana leaves and control leaves (tubes 1 & 2 represent the solution before fixing and tissues in fixing solution, respectively from healthy leaves whereas 3& 4 represent the solution and tissues from infected leaves, respectively, G tube is the GUS assay buffer (B.) Activity of reporter genes in C. macrophylla (a.) Picture of peeled phloem bark pieces of C. macrophylla infected with construct CTV33-23-BY-GFP-HC-GUS-51 under a fluorescent stereoscope (b.) Peeled bark phloem pieces GUS activity in infected and healthy C. macrophylla plants (tubes 1 & 2 represent the solution and tissues in fixing solution from healthy leaves whereas 3 & 4 represent the solution and tissues from infected leaves, respectively.

FIG. 15 Bimolecular Fluorescence complementation (BiFC) prove of concept. (A) Schematic representation of CTVΔ Cla 333R (Gowda et al., 2001, Satyanarayana et al., 2003) replicon and its modification to create expression replicons: (a.) Insertion of both BiFC genes between p23 and 3′NTR giving rise to CTVp333R-23-BYbJunN-GbFosC and the controls with one gene behind p23, CTVp333R-23-BYbJunN(b.) or CTVp333R-23-GbFosC(c.). (B) Northern blot hybridization analysis of transfected protoplast with CTVp333R-23-BYbJunN-GbFosC (Lane a.), CTVp333R-23-BYbJunN (Lane c.) and CTVp333R-23-GbFosC (Lane b.). (C) Fluorescence of a transfected protoplast when pictured under a stereoscope (Upper) or a laser scanning confocal microscope (lower) indicating the fluorescence from the nucleus.

FIG. 16 BiFC gene replacement of p13 to produce CTV based expression vectors. (A) Schematic representation of CTV9RΔp33and modification to produce vector CTV33-Δ13-BYbJunN-GbFosC-76 and the control vectors CTV33-23-G-bFosC-98 and CTV33-23-BY-bJunN-97 (insertion behind p23 nts 19020-19021). (B) Representative sample of N. benthamiana fluorescence in systemically infected plants.

FIG. 17 CTV based expression vector built to simultaneously express two genes from two controller elements. (A) Schematic representation of CTV9RΔp33and its modification to produce expression vectors CTV33-23-BYbJunN-GbFosC-59 and CTV33-Δ13-BYbJunN-23-GbFosC-67. (B) Northern blot hybridization analysis of the RNA transfected protoplast with the wild type virus (WT,T), two clones of CTV33-Δ13-BYbJunN-23-GbFosC-67(C67,T1 and T2) and two clones of CTV33-23-BY-bJunN-Gb-FosC-59 (C59, T3 and T4) probed with 3′NTR+p23 (Satyanarayana et al., 1999). (C) Fluorescence of N. benthamiana plant parts under a flourescent stereo microscope (CTV33-23-BY-bJunN-Gb-FosC-59=a., b., c. and d; CTV33-Δ13-BYbJunN-23-GbFosC-67=e.) (a.) bud (b.) Corolla, (c.) systemic leaves, (d.) peeled bark phloem pieces and (e.) infiltrated leaf

FIG. 18 CTV based expression vector built to simultaneously express two genes from two controller elements. (A) Schematic representation of CTV9RΔp33and its modification to produce expression vectors CTV33-Δ13-BYGUS-23-GGFP-71. (B) Northern blot hybridization analysis of the RNA transfected protoplast with the wild type virus (WT) and the CTV33-Δ13-BYGUS-23-GGFP-71 (C71) expression vector probed with 3′NTR +p23 (Satyanarayana et al., 1999). (C) Biological activity of reporter genes in N. benthamiana and Citrus. N. benthamiana plant under white light (a.) and hand held UV light (b.). (c.) GUS activity from healthy (tube 1 (assay solution) &2 (tissue) and infected N. benthamiana (tube 3 (assay solution) and tube 4 (tissue). (d.) Peeled bark phloem pieces under fluorescent microscope and (e.) GUS assay activity in citrus similar to (c.)

FIG. 19 Western blot analysis of the different constructs in citrus to evaluate the expression of GFP and GUS. (A) GFP and CP antibody used to determine the level of expression of GFP relative to CP in citrus 708 plant infected with Δp33CTV9R (Tatineni et al., 2008), 1808 plant infected with BCN5 (Folimonov et al., 2007), 1916 plant infected with CTV33-23-G-GFP-40, 1874 plant infected with CTV33-23-BY-GFP-37, 1934, 1935, 1937 infected with CTV33-13-BY-GFP-69, 1931 and 1939 infected with construct CTV33-Δ13-G-GFP-65 and CTV33-Δ13-B-GFP-66, respectively. (B) GUS and CP antibody used to determine the level of expression of GUS relative to CP in citrus 2084, 2085, 2086, 2087 plants infected with construct CTV33-Δ13-BYGUS-61, 2132 plant infected with construct CTV33-23-BYGUS-60, 2096 plant infected with expression vector CTV33-Δ13-BYGFP-NIa-GUS-78, E=empty well and buffer=−iveC.

FIG. 20 CTV based expression vector built to simultaneously express four genes from four controller elements. (A) A schematic representation of CTV9R. (B) Modification of CTV9R to create expression vector CTVΔ13-BRFP-GbFosC-BYbJunN-CTMVCP-118 which expresses 4 genes from different locations within the CTV genome. The first gene is the red fluorescent protein gene (tagRFP) expressed from between the minor and major coat proteins under the control of the Beet yellows virus (BYV) coat protein controller element (CP-CE), the second and third genes are the truncated mammalian transcription factors bFos and bJun fused to the C and N terminus of EYFP (Hu et al., 2002) under the control of Grape vine leaf roll associated virus-2 (GLRaV-2) and Beet yellow stunt virus (BYSV) CP-CE respectively replacing the p13 gene and the fourth gene is the CP of TMV expressed from behind p23 under the control of the duplicated major CP-CE of CTV.

FIG. 21CTV based expression vector built to simultaneously express three genes from three controller elements. (A) A schematic representation of CTV9R. (B) Modification of CTV9R to create expression vector CTVΔ13-GbFosC-BYbJunN-CTMVCP-129 which expresses 3 genes from different locations within the CTV genome. The first and second genes are the truncated mammalian transcription factors bFos and bJun fused to the C and N terminus of EYFP (Hu et al., 2002) under the control of Grape vine leaf roll associated virus-2 (GLRaV-2) and Beet yellow stunt virus (BYSV) CP-CE respectively replacing the p13 gene and the fourth gene is the CP of TMV expressed from behind p23 under the control of the duplicated major CP-CE of CTV.

FIG. 22 CTV based expression vector built to simultaneously express three genes from three controller elements. (A) A schematic representation of CTV9R. (B) Modification of CTV9R to create expression vector CTV-BRFP-BYGFP-CTMVCP-117 which expresses 3 genes from different locations within the CTV genome. The first gene is the red fluorescent protein gene (tagRFP) expressed from between the minor and major coat proteins under the control of the Beet yellows virus (BYV) coat protein controller element (CP-CE), the second gene is the Green fluorescent protein (GFPC3) under the control of Beet yellow stunt virus (BYSV) CP-CE inserted between p13-p20 gene and the third gene is the CP of TMV expressed from behind p23 under the control of the duplicated major CP-CE of CTV.

FIG. 23 CTV based expression vector built to simultaneously express three genes from three controller elements. (A) A schematic representation of CTV9R. (B) Modification of CTV9R to create expression vector CTV-BASL-BYPTA-CP7-119 which expresses 3 genes from different locations within the CTV genome. The first gene is a lectin from Allium sativum (ASL) expressed from between the minor and major coat proteins under the control of the Beet yellows virus (BYV) coat protein controller element (CP-CE), the second gene is an agglutinin from Pinellia ternata (PTA) under the control of Beet yellow stunt virus (BYSV) CP-CE inserted between p13-p20 gene and the third gene is an antimicrobial peptide from Tachypleus tridentatus (P7) expressed from behind p23 under the control of the duplicated major CP-CE of CTV.

FIG. 24 CTV based expression vector built to simultaneously express three genes from three controller elements. (A) A schematic representation of CTV9R. (B) Modification of CTV9R to create expression vector CTV-BASL-BYPTA-CP10-120 which expresses 3 genes from different locations within the CTV genome. The first gene is a lectin from Allium sativum (ASL) expressed from between the minor and major coat proteins under the control of the Beet yellows virus (BYV) coat protein controller element (CP-CE), the second gene is an agglutinin from Pinellia ternata (PTA) under the control of Beet yellow stunt virus (BYSV) CP-CE inserted between p13-p20 gene and the third gene is an antimicrobial peptide from Sus scorfa (P10) expressed from behind p23 under the control of the duplicated major CP-CE of CTV.

FIG. 25 CTV based expression vector built to simultaneously express three genes from three controller elements. (A) A schematic representation of CTV9R. (B) Modification of CTV9R to create expression vector CTV-BASL-BYP10-CP7-131 which expresses 3 genes from different locations within the CTV genome. The first gene is a lectin from Allium sativum (ASL) expressed from between the minor and major coat proteins under the control of the Beet yellows virus (BYV) coat protein controller element (CP-CE), the second gene is an antimicrobial peptide from Sus scorfa (P10) under the control of Beet yellow stunt virus (BYSV) CP-CE inserted between p13-p20 gene and the third gene is a second antimicrobial peptide from Tachypleus tridentatus (P7) expressed from behind p23 under the control of the duplicated major CP-CE of CTV.

FIG. 26 CTV based expression vector built to simultaneously express three genes from three controller elements. (A) A schematic representation of CTV9RΔp33. (B) Modification of CTV9R Δp33to create expression vector CTV33-BGFP-BYGUS-GTMVCP-79 which expresses 3 genes from different locations within the CTV genome. The first gene is a green fluorescent protein expressed from between the minor and major coat proteins under the control of the Beet yellows virus (BYV) coat protein controller element (CP-CE), the second gene is a β-Glucuronidase (GUS) gene from Escherichia coli under the control of Beet yellow stunt virus (BYSV) CP-CE inserted between p13-p20 gene and the third gene is the CP of TMV expressed from behind p23 under the control of Grape vine leaf roll associated virus-2 (GLRaV-2) CP-CE.

FIG. 27 CTV based expression vector built to simultaneously express four genes from four controller elements. (A) A schematic representation of CTV9RΔp33. (B) Modification of CTV9RΔp33to create expression vector CTV33-BGFP-GbFosC-BYbJunN-81 which expresses 3 genes from different locations within the CTV genome. The first gene is the green fluorescent protein gene (GFPC3) expressed from between the minor and major coat proteins under the control of the Beet yellows virus (BYV) coat protein controller element (CP-CE), the second and third genes are the truncated mammalian transcription factors bFos and bJun fused to the C and N terminus of EYFP (Hu et al., 2002) under the control of Grape vine leaf roll associated virus-2 (GLRaV-2) and Beet yellow stunt virus (BYSV) CP-CE respectively. The bFosC gene is inserted behind p23 gene.

FIG. 28 CTV based expression vector built to simultaneously express four genes from four controller elements. (A) A schematic representation of CTV9RΔp33. (B) Modification of CTV9RΔp33to create expression vector CTV33-Δ13-BGFP-BYbJunN-GbFosC-82 which expresses 3 genes from different locations within the CTV genome. The first gene is the green fluorescent protein gene (GFPC3) expressed from between the minor and major coat proteins under the control of the Beet yellows virus (BYV) coat protein controller element (CP-CE), the second gene is the truncated mammalian transcription factor bJun to the N terminus of EYFP (bJunN) (Hu et al., 2002) under the control of Beet yellow stunt virus (BYSV) CP-Ce replacing the p13 gene of CTV and the third gene is the truncated mammalian transcription factor bFos fused to the C-terminus of EYFP (bFosC) under the control of Grape vine leaf roll associated virus-2 (GLRaV-2) CP-CE inserted behind p23.

FIG. 29 Negative staining Electron microscopy pictures from leaf dips of infiltrated N. benthamiana leaves. (A) Leaf dips from infiltrated N. benthamiana leaves with construct CTV33-BGFP-BYGUS-GTMVCP-79 reveals the formation of CTV vector virions and TMV pseudo virions indicating the expression of the TMV coat protein gene. (B) Leaf dip from Infiltrated N. benthamiana leaves with construct CTV33-Δ13-BGFP-BYbJunN-GbFosC-82 reveals the formation of virions.

FIG. 30 provides a map of the CTV genome and a CTV-based expression vector.

### BRIEF DESCRIPTION OF THE SEQUENCE LISTING

Spinach Defensin Sequences:

SEQ ID NO: 1 illustrates an amino acid sequence of a spinach (Spinacia oleracea) defensin (SoD2) according to a specific example embodiment of the disclosure;

SEQ ID NO: 2 illustrates an amino acid sequence of a spinach (Spinacia oleracea) defensin (SoD7) according to a specific example embodiment of the disclosure;

SEQ ID NO: 3 illustrates a GenScript-optimized nucleic acid sequence for expression of a spinach (Spinacia oleracea) defensin (SoD2) according to a specific example embodiment of the disclosure;

SEQ ID NO: 4 illustrates a GenScript-optimized nucleic acid sequence for expression of a spinach (Spinacia oleracea) defensin (SoD7) according to a specific example embodiment of the disclosure;

SEQ ID NO: 5 illustrates a CODA-optimized nucleic acid sequence for expression of a spinach (Spinacia oleracea) defensin (SoD2) according to a specific example embodiment of the disclosure;

SEQ ID NO: 6 illustrates a CODA-optimized nucleic acid sequence for expression of a spinach (Spinacia oleracea) defensin (SoD7) according to a specific example embodiment of the disclosure;

SEQ ID NO: 7 illustrates an amino acid sequence of a chimeric peptide comprising a PR-1b signal peptide and a spinach (Spinacia oleracea) defensin (SoD2) according to a specific example embodiment of the disclosure;

SEQ ID NO: 8 illustrates an amino acid sequence of a chimeric peptide comprising a PR-1b signal peptide and a spinach (Spinacia oleracea) defensin (SoD7) according to a specific example embodiment of the disclosure;

SEQ ID NO: 9 illustrates a chimeric nucleic acid sequence comprising a nucleic acd sequence encoding a PR-1b signal peptide and a GenScript-optimized nucleic acid sequence for expression of a spinach (Spinacia oleracea) defensin (SoD2) according to a specific example embodiment of the disclosure;

SEQ ID NO: 10 illustrates a chimeric nucleic acid sequence comprising a nucleic acd sequence encoding a PR-1b signal peptide and a GenScript-optimized nucleic acid sequence for expression of a spinach (Spinacia oleracea) defensin (SoD7) according to a specific example embodiment of the disclosure;

SEQ ID NO: 11 illustrates a chimeric nucleic acid sequence comprising a nucleic acd sequence encoding a PR-1b signal peptide and a CODA-optimized nucleic acid sequence for expression of a spinach (Spinacia oleracea) defensin (SoD2) according to a specific example embodiment of the disclosure;

SEQ ID NO: 12 illustrates a chimeric nucleic acid sequence comprising a nucleic acd sequence encoding a PR-1b signal peptide and a CODA-optimized nucleic acid sequence for expression of a spinach (Spinacia oleracea) defensin (SoD7) according to a specific example embodiment of the disclosure;

SEQ ID NO: 13 illustrates an expression cassette comprising a nucleic acid sequence encoding a PR-1b signal peptide and a GenScript-optimized nucleic acid sequence for expression of a spinach (Spinacia oleracea) defensin (SoD2) according to a specific example embodiment of the disclosure;

SEQ ID NO: 14 illustrates an expression cassette comprising a nucleic acid sequence encoding a PR-1b signal peptide and a GenScript-optimized nucleic acid sequence for expression of a spinach (Spinacia oleracea) defensin (SoD7) according to a specific example embodiment of the disclosure;

SEQ ID NO: 15 illustrates an expression cassette comprising a nucleic acid sequence encoding a PR-1b signal peptide and a CODA-optimized nucleic acid sequence for expression of a spinach (Spinacia oleracea) defensin (SoD2) according to a specific example embodiment of the disclosure;

SEQ ID NO: 16 illustrates an expression cassette comprising a nucleic acid sequence encoding a PR-1b signal peptide and a CODA-optimized nucleic acid sequence for expression of a spinach (Spinacia oleracea) defensin (SoD7) according to a specific example embodiment of the disclosure;

SEQ ID NO: 17 illustrates an expression control sequence (CaMV 35S promoter) according to a specific example embodiment of the disclosure;

SEQ ID NO: 18 illustrates an untranslated region (TEV 5′UTR) according to a specific example embodiment of the disclosure;

SEQ ID NO: 19 illustrates an expression control sequence (CaMV 35S terminator) according to a specific example embodiment of the disclosure;

SEQ ID NO: 20 illustrates a nucleic acid sequence of a primer designated Zn5 according to a specific example embodiment of the disclosure;

SEQ ID NO: 21 illustrates a nucleic acid sequence of a primer designated Zn6 according to a specific example embodiment of the disclosure;

SEQ ID NO: 22 illustrates a nucleic acid sequence of a primer designated Fcp according to a specific example embodiment of the disclosure;

SEQ ID NO: 23 illustrates a nucleic acid sequence of a primer designated Rcp according to a specific example embodiment of the disclosure;

SEQ ID NO: 24 illustrates a nucleic acid sequence of a primer designated GUSF according to a specific example embodiment of the disclosure;

SEQ ID NO: 25 illustrates a nucleic acid sequence of a primer designated GUSR according to a specific example embodiment of the disclosure;

SEQ ID NO: 26 illustrates an amino acid sequence of a chimeric peptide comprising a modified PR-1b signal peptide and a GenScript-optimized nucleic acid sequence having a single deletion for expression of a spinach (Spinacia oleracea) defensin (SoD2) according to a specific example embodiment of the disclosure;

SEQ ID NO: 27 illustrates a chimeric nucleic acid sequence comprising a nucleic acid sequence encoding a modified PR-1b signal peptide and a GenScript-optimized nucleic acid sequence having a single deletion for expression of a spinach (Spinacia oleracea) defensin (SoD2) according to a specific example embodiment of the disclosure;

SEQ ID NO: 28 illustrates a core amino acid sequence of a defensin according to a specific example embodiment of the disclosure;

SEQ ID NO: 29 illustrates a nucleic acid sequence for expression of a core defensin according to a specific example embodiment of the disclosure;

SEQ ID NO: 30 illustrates a DNA 2.0-optimized nucleic acid sequence for expression of a spinach (Spinacia oleracea) defensin (SoD2) according to a specific example embodiment of the disclosure; and

SEQ ID NO: 31 illustrates a DNA 2.0-optimized nucleic acid sequence for expression of a spinach (Spinacia oleracea) defensin (SoD7) according to a specific example embodiment of the disclosure.

### DETAILED DESCRIPTION

Embodiments of the invention provide citrus plants resistant to citrus Huanglongbing (ex greening) caused by Candidatus Liberibacter asiaticus (Las) and bacterial canker caused by (Xanthomonas axonopodis pv. citri) (Xac) by using a citrus tristeza virus (“CTV”) based vector comprising spinach defensin genes. Innoculation of the citrus plants with the vectors of the present invention protect the citrus plants against citrus greening disease and/or bacterial canker.

Ongoing efforts have been underway to create virus-based vectors for citrus trees based on Citrus tristeza virus (CTV). The CTV vector is described in U.S. application Ser. No. 13/624,294 and is incorporated herein in its entirety. Spinach defensin genes are described in U.S. application Ser. No. 13/751,936 and is incorporated herein in its entirety.

CTV Vectors

CTV has the largest reported RNA of a plant virus of approximately 20 kb (Karasev et al., 1995; Pappu et al., 1994). It has two conserved gene blocks associated with replication and virion formation (Karasev, 2000). The replication gene block occupies the 5′ half of the genome. Its proteins are expressed from the genomic RNA via a poly protein strategy with a +1 ribosomal frame shift to occasionally express the RNA dependent RNA polymerase (Karasev et al., 1995). The filamentous virions of CTV are encapsidated by two coat proteins, with the major coat protein (CP) encapsidating about 97% of the virion and the 5′˜700 nts encapsidated by the minor coat protein (CPm) (Satyanarayana et al., 2004). Virion formation is a complex process requiring two proteins (Hsp70h and p61) in addition to the coat proteins (Satyanarayana et al., 2000, 2004; Tatineni et al., 2010). These four genes as well as the 6 remaining genes are differentially expressed via a nested set of 3′ co-terminal sub genomic (sg) RNAs (Hilf et al., 1995). Upstream of each ORF there is a controller element (CE) that determines the transcription level (Gowda et al., 2001). Levels of transcription are also associated with the +1 transcription start site (Ayllon et al., 2003), the presence of a non-translated region upstream of the ORF (Gowda et al., 2001), and the closeness of the ORF to the 3′ terminus (Satyanarayana et al., 1999).

The first generations of CTV vector examined three different strategies that were fusion of the CP gene, insertion of an extra gene, and replacement of the p13 ORF (Folimonov et al., 2007). Replacement of the p13 ORF and fusion to the coat protein ORF did not result in effective vectors, but the addition of an extra gene resulted in viable vectors that produce relative large amounts of foreign gene and were stable in citrus trees for years. However, the first efforts in designing vectors based on CTV examined only a few of the many possibilities for expressing foreign genes in this large virus. In this work, Dawson's lab attempted to examine the limitations of CTV to be manipulated into a vector. Dawson's lab examined whether the virus allowed insertions in different positions within the genome and which resulted in maximal expression with different sizes of inserts. Dawson's lab also examined whether different fusion strategies with different viral genes are viable and whether multiple foreign genes can be expressed. The CTV constructs disclosed herein are amazingly tolerant to manipulation at several positions within the genome giving a multitude of different vector strategies that are viable.

Once citrus is infected with a CTV vector containing a foreign gene, it is easy to move the vector to other citrus trees by grafting. However, a limitation of the CTV vector system is the difficulty of initially getting citrus infected with new vector constructs. Directly inoculating citrus from the cDNA clones, either by agro-inoculation, particle bombardment, or mechanical inoculation with RNA transcripts is extremely difficult and unpredictable (Gowda et al., 2005; Satyanarayana et al., 2001). An alternative has been to inoculate with virions purified from Nicotiana benthamiana protoplasts (Folimonov et al., 2007; Robertson et al., 2005; Satyanarayana et al., 2001; Tatineni et al., 2008). However, infection of only approximately 0.01-0.1% of protoplasts with in vitro transcribed RNA has been achieved (Satyanarayana et al., 2001). Yet, since virions are much more infectious to the protoplasts than RNA (Navas-Castillo et al., 1997), Dawson's lab was able to amplify the infection by sequential passage in protoplasts (Folimonov et al., 2007; Robertson et al., 2005; Satyanarayana et al., 2001; Tatineni et al., 2008). Although workable, this is an extremely difficult system. Dawson's lab is now able to agro-inoculate N. benthaminana plants that result in systemic infection. This result allows analysis of the vector constructs more quickly in these plants and provides copious amounts of recombinant virus for inoculation of citrus.

According to one embodiment, the invention pertains to a CTV viral vector engineered to comprise a gene cassette comprising a polynucleotide encoding a spinach defensin peptide(s). The gene cassette is located at a targeted position on the CTV genome. In a more specific embodiment, the CTV viral vector is engineered such that the gene cassette is positioned at CTV genome regions p13-p20, p20-p23 or p23-3′NTR. In other embodiments, the CTV viral vector is engineered to include multiple genes at one or multiple positions. It is shown herein that CTV viral vectors can successfully be engineered to include up to 3 or at least 4 genes that are expressible by the vector, while maintaining the proper function and infectivity of the vector.

In related embodiments, the invention pertains to a plant that includes at least one cell transfected with the CTV viral vector engineered to comprise a gene cassette comprising a polynucleotide encoding a spinach defensin peptide(s), the CTV viral vector engineered such that one or more gene cassettes are positioned at CTV genome regions p13-p20, p20-p23 or p23-3′NTR. Other related embodiments pertain to methods of expressing at least one spinach defensin peptide(s) in a plant by infecting the plant with the specified vector.

In a further embodiment, the invention is directed to a CTV viral vector engineered to comprise at least one gene cassette that includes a polynucleotide encoding a spinach defensin peptide(s), wherein the CTV viral vector engineered such that the gene cassette is inserted in place of the CTV p13 gene. In related embodiments, the invention pertains to a plant that includes at least one cell transfected with the CTV viral vector or to methods of expressing the spinach defensin peptide(s) in a plant by infecting the plant with the specified vector.

In another embodiment, the invention relates to a CTV viral vector engineered to comprise at least one gene cassette comprising a polynucleotide encoding spinach defensin peptide(s) and IRES sequence conjugated thereto. In related embodiments, the invention pertains to a plant that includes at least one cell transfected with the CTV viral vector or to methods of expressing the spinach defensin peptide(s) in a plant by infecting the plant with the specified vector.

In further embodiments, the invention relates to a CTV viral vector engineered to comprise a gene cassette comprising a polynucleotide sequence with continuous amino acid codons extending from the p23 ORF encoding a protease with cleavage sites on each side plus a spinach defensin peptide(s). In related embodiments, the invention pertains to a plant that includes at least one cell transfected with the CTV viral vector or to methods of expressing the spinach defensin peptide(s) in a plant by infecting the plant with the specified vector.

In further embodiments, the polynucleotide further comprises a sequence encoding a first control element upstream of said first spinach defensin peptide(s), a second sequence encoding a protease with cleavage sites engineered on each side, and a sequence encoding a second spinach defensin peptide(s).

According to another embodiment, the invention is directed to CTV viral vector engineered to comprise a first gene cassette comprising a polynucleotide sequence encoding a first spinach defensin peptide(s) and a first controller element upstream of said first spinach defensin peptide(s) encoding sequence; and a second gene cassette comprising a polynucleotide sequence encoding a second spinach defensin peptide(s) and a second control element upstream of said second spinach defensin peptide(s) encoding sequence. Optionally, the CTV viral vector further comprises a third gene cassette comprising a polynucleotide sequence encoding a third spinach defensin peptide(s) and a third controller element upstream of said third spinach defensin peptide(s) encoding sequence; and a fourth gene cassette comprising a polynucleotide sequence encoding a fourth spinach defensin peptide(s) and a fourth controller element upstream of said fourth spinach defensin peptide(s) encoding sequence. Those skilled in the art will appreciate that additional gene cassettes can be added to the vector so long as function and infectivity of the vector is maintained. In related embodiments, the invention pertains to a plant that includes at least one cell transfected with the CTV viral vector or to methods of expressing the spinach defensin peptide(s) in a plant by infecting the plant with the specified vector.

Examples of controller elements (CE) useful in accordance with the teachings herein include but are not limited to controller elements homologous to CTV or heterologous control elements. Heterologous controller elements include, but are not limited to, coat protein controller elements (CP-CEs) of three closteroviruses: Beet yellows virus (BYV) (94 nts from 13547-13640 Genbank accession #AF190581) (Peremyslov et al., 1999), Beet yellow stunt virus (BYSV) (101 nts from 8516-8616 Genbank accession #U51931) (Karasev et al., 1996) and Grape vine leaf roll associated virus-2 (GLRaV-2) (198 nts from 9454-9651 Genbank accession #DQ286725). It will be evident to those skilled in the art, in view of the teachings herein, that other controller elements may be implemented, and in particular control elements having strong promoter like activity.

These and other embodiments are further described below and encompassed within the appended claims.

Materials and Methods for Examples 1-8 Below

Plasmids Construction

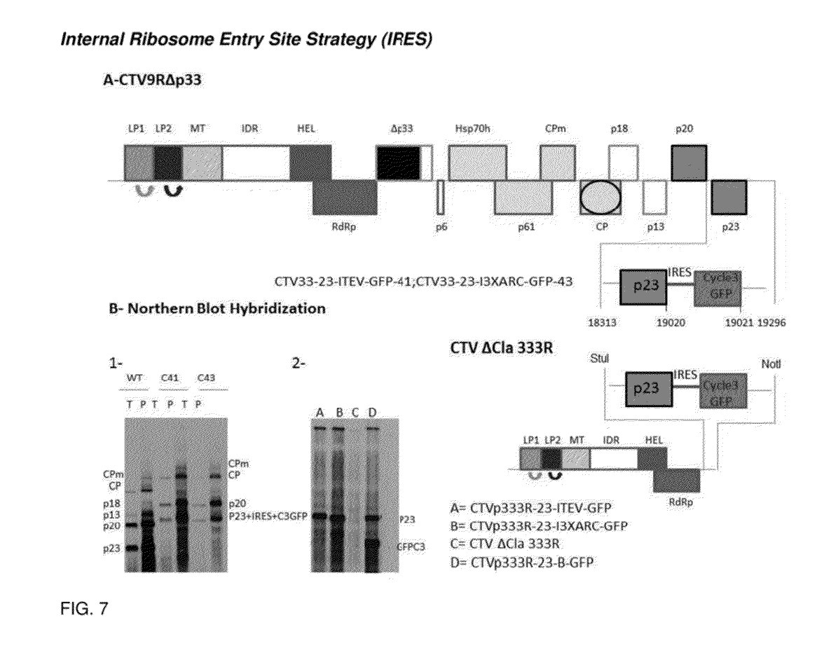

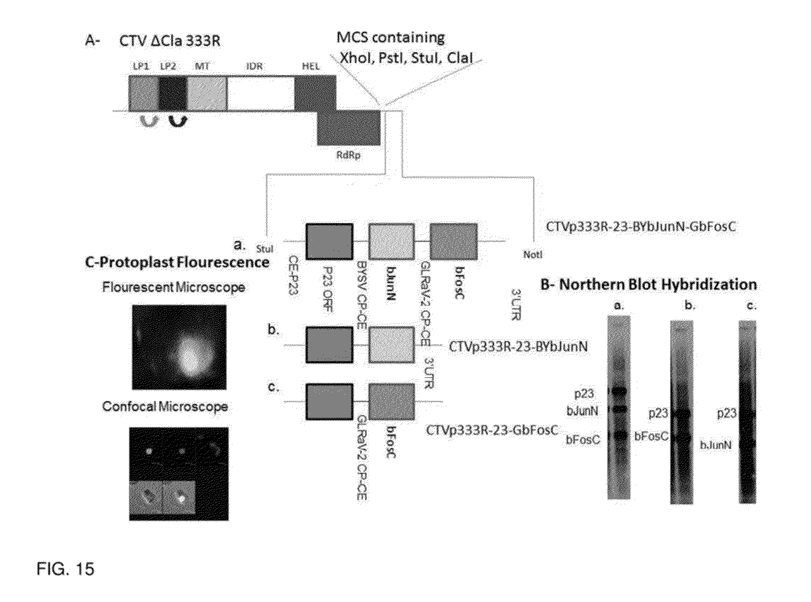

pCTV9RΔp33and pCTVΔCla 333R (Gowda et al., 2001; Satyanarayana et al., 1999, 2000, 2003; Tatineni et al., 2008) were used as base plasmids for developing all expression vectors that were used in the protoplast reverse genetics system. The numbering of the nucleotides (nts) is based on the full length T36 clone (Genbank Accession #AY170468) (Satyanarayana et al., 1999, 2003). CTVp333R-23-ITEV-GFP and CTVp333R-23-I3XARC-GFP (FIG. 7A) were created by fusing 5′ non translated region (NTR) of Tobacco etch virus (TEV) (nucleotides (nts) 2-144 Genbank accession #DQ986288) (Carrasco et al., 2007) and 3xARC-1 (Active ribosome complementary sequence) (Akergenov et al., 2004) behind the p23 stop codon (between nts19020-19021 in full length T36 clone) using overlap extension polymerase chain reaction (PCR) (Horton et al., 1989). For creating expression vectors by gene addition and/or substitution at different locations, heterologous controller elements (CE) were selected from coat protein controller elements (CP-CEs) of three closteroviruses: Beet yellows virus (BYV) (94 nts from 13547-13640 Genbank accession #AF190581) (Peremyslov et al., 1999), Beet yellow stunt virus (BYSV) (101 nts from 8516-8616 Genbank accession #U51931) (Karasev et al., 1996) and Grape vine leaf roll associated virus-2 (GLRaV-2) (198 nts from 9454-9651 Genbank accession #DQ286725) to drive the ORFs for cycle 3 GFP (GFP) (Chalife et al., 1994; Crameri et al., 1996), β-Glucuronidase (GUS) ORF of Eisherchia coli, bFosYC155-238 (bFosC), bJunYN1-154 (bJunN). CTVp333R-23-BYbJunN-GbFosC, CTVp333R-23-BYbJunN, CTVp333R-23-GbFosC (FIG. 15A) were created by overlap extension PCR from plasmids pBiFC-bFosYC155 and pBiFC-bJunYN155 (Hu et al., 2002) and CTV9R (Satyanarayana et al., 1999; 2003). Since two NotI sites exist within the bimolecular fluorescence genes (BiFC), the overlap extension PCR products were digested partially by NotI restriction endonuclease. The PCR products were introduced into a StuI and NotI digested pCTVΔCla 333R (FIGS. 7A & 3-15A).

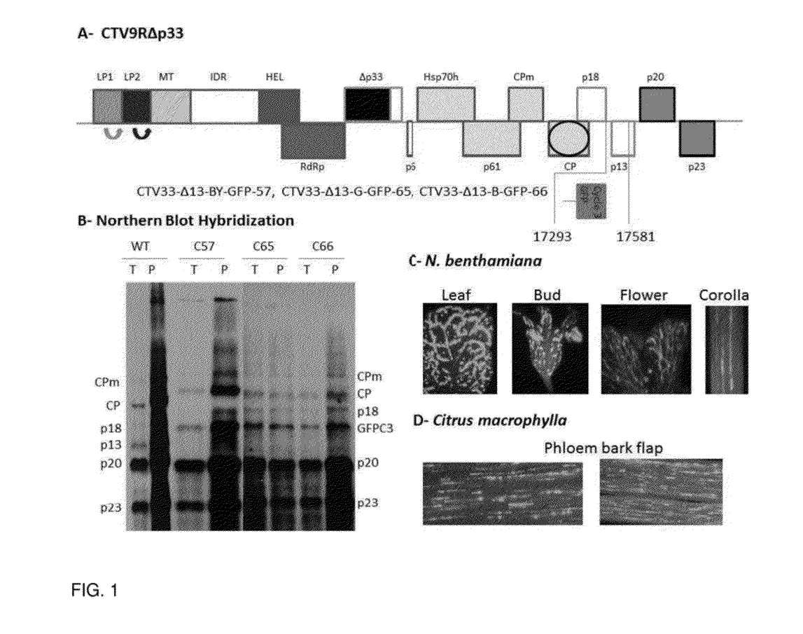

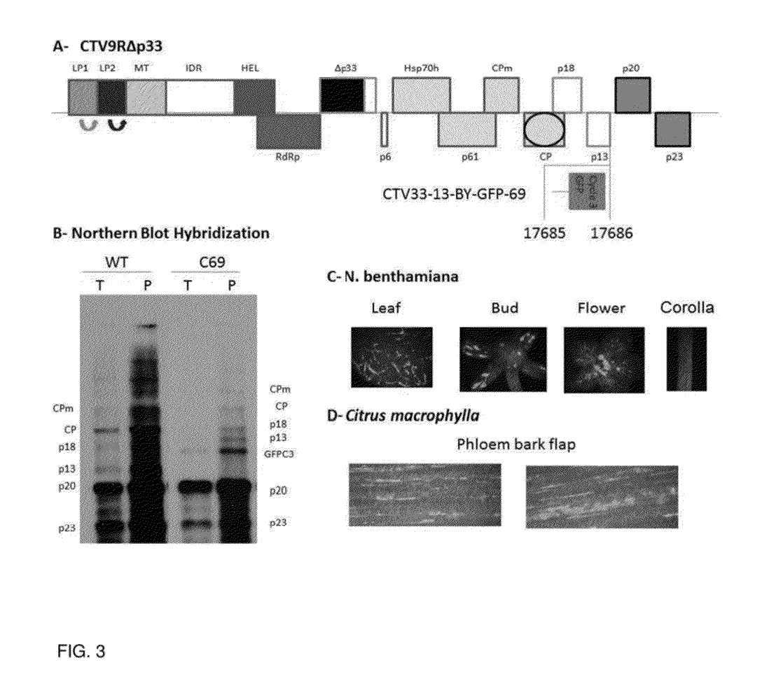

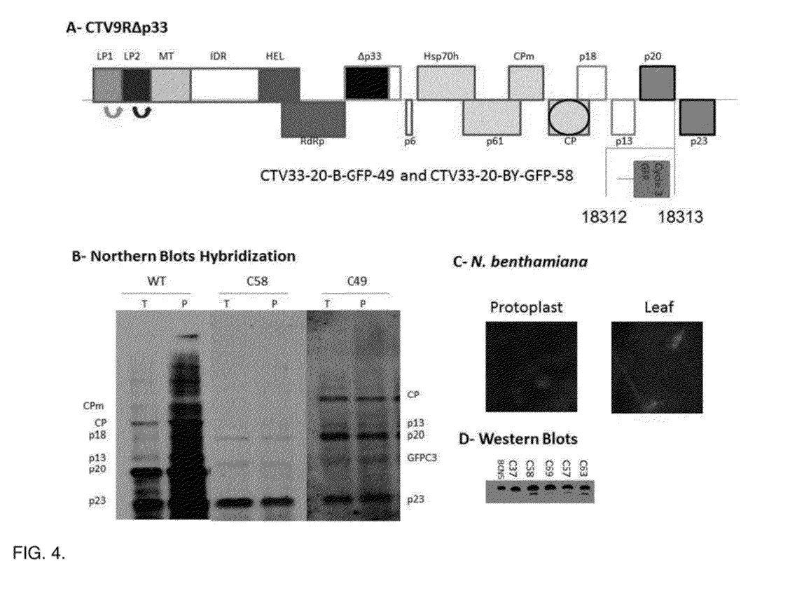

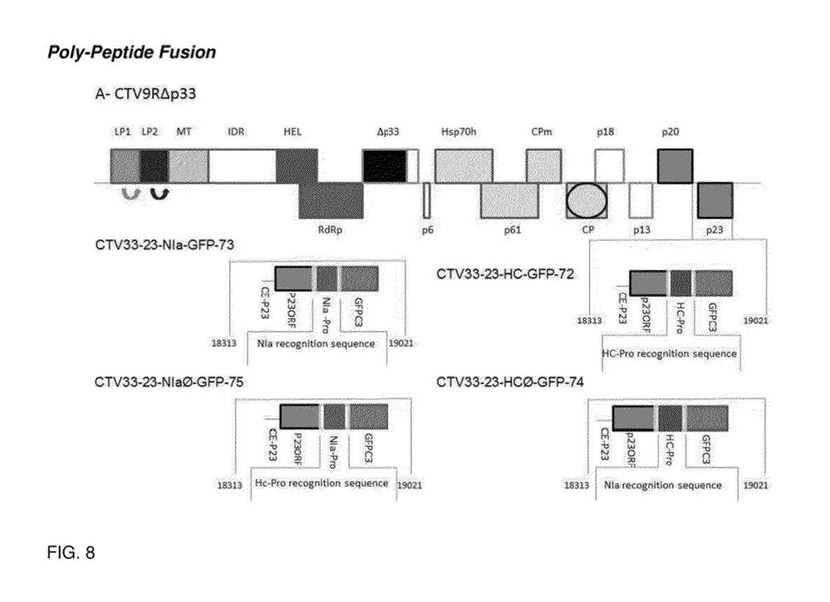

The expression vectors created in pCTV9RΔp33were introduced into the CTV genome by digesting the plasmid with PstI (nts 17208-17213) and NotI or StuI (introduced behind 19,293 the final CTV nucleotide). Overlap extension PCR (Horton et al., 1989) was used to introduce the appropriate genes at the different locations. Replacement of the p13 gene was done by deletion of nts 17293-17581 in the p13 ORF and (CE) by overlap extension PCR (FIGS. 3-1A, 3-2A, 3-11A, 3-16A, 3-17A & 3-18A). Similarly, insertion between p13 and p20 (nts #17685-17686) (FIG. 3A), p20-p23 (nts #18312-18313) (FIG. 4A) and p23-3′NTR (nts #19020-19021) (FIGS. 3-5A, 3-6A, 3-13A, 3-16A, 3-17A & 3-18A) were done by overlap extension PCR. A hybrid gene created by fusing the GFP ORF (Chalife et al., 1994; Crameri et al., 1996) and GUS ORF separated by the HC-Pro protease motif (nts 1966-2411 Genbank accession #M11458) (Allison et al., 1985; Carrington et al., 1989) and its recognition sequence fused to the N terminus of GUS (ATGAAAACTTACAATGTTGGAGGGATG (nts 2412-2438 Genbank accession #M11458) (Allison et al., 1985; Carrington et al., 1989) (Amino acid sequence (A.A.) MKTYNVG↓GM) (arrow indicate processing site) and C terminus of GFP (ATGAAGACCTATAACGTAGGTGGCATG) was created and inserted behind p23 (FIG. 13A) or as replacement of p13 (FIG. 3-11A) under different controller elements. A similar hybrid gene was created by using the NIa protease motif of TEV (nts 6270-6980 Genbank accession #M11458) (Allison et al., 1985) and its recognition sequence (GAGAATCTTTATTTTCAGAGT (nts 8499-8519 Genbank accession #M11458) (A.A. ENLYFQ↓S) (arrow indicate processing site) (Carrington and Dougherty, 1988) at C terminus of GFP and GAAAACCTATACTTCCAATCG at N terminus of GUS). The redundancy of the amino acid genetic code was used to eliminate complete duplication of the nucleotide sequences of the recognition motifs. A similar strategy was used to create a hybrid gene between p23 ORF and GFP ORF in construct CTV33-23-HC-GFP-72 and CTV33-23-NIa-GFP-73 (FIG. 8). Switching the recognition motif of the proteases generated control vectors CTV33-23-HCØ-GFP-74 and CTV33-23-NIaØ-GFP-75 (FIG. 8).

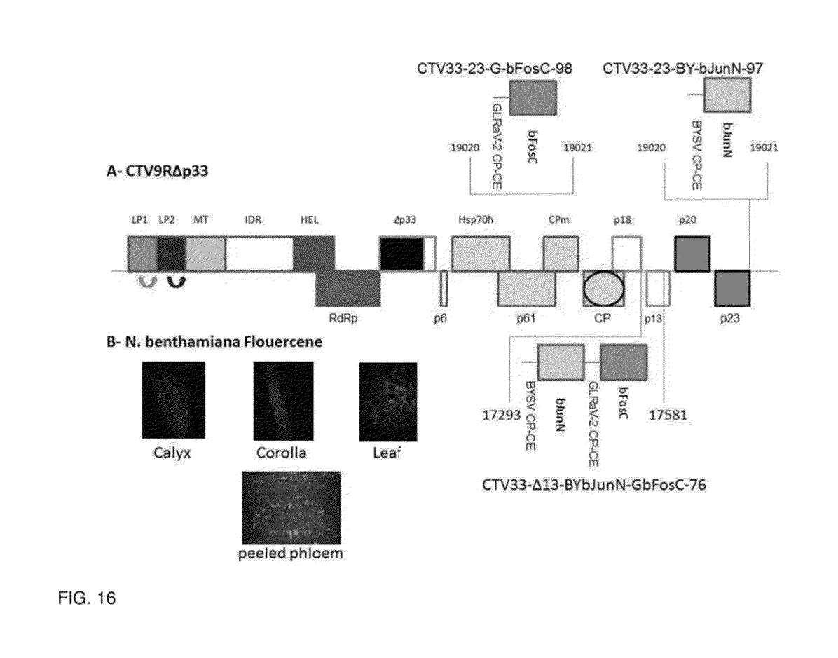

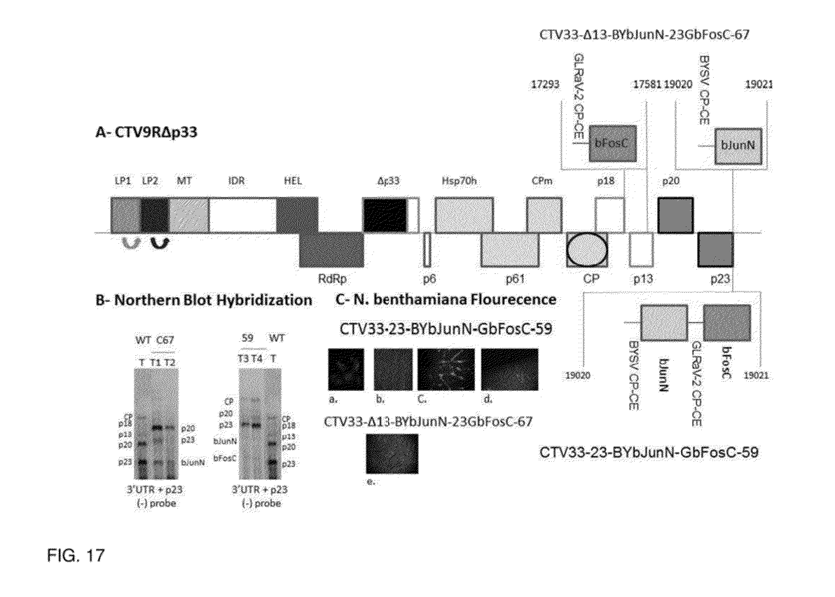

The binary plasmid pCAMBIACTV9R (Gowda et al., 2005) was modified to eliminate the p33 gene by deleting nts 10858-11660 (Satyanarayana et al., 2000; Tatineni et al., 2008) and introducing a SwaI site behind the ribozyme engineered based on subterranean clover mottle virusoid (Turpen et al., 1993). PCR products amplified from the expression vectors in the pCTV9RΔp33 back-bone were introduced into the modified binary plasmid pCAMBIACTV9RΔp33digested with PstI (Forward primer C-749) and SwaI (Reverse primer C-1894). When introducing the bimolecular fluorescence complementation (BiFC) genes into constructs CTV33-23-BYbJunN-GbFosC-59 (FIG. 17), CTV33-Δ13-BYbJunN-23-GbFosC-67 (FIG. 17) , CTV33-Δ13-BYbJunN-GbFosC-76 (FIG. 16), CTV33-23-GbFosC-98 (FIG. 16) and CTV33-23-BYbJunN-97 (FIG. 16) a primer was used switching the PstI to the compatible NsiI (primer C-2085) for ease of cloning (the bFosC gene sequence contains one PstI site while the bJunN gene sequence contains two PstI sites). Preliminary screening for the right inserts in the different expression vectors was done by restriction digestion using the appropriate enzymes. The junctions where the foreign genes were introduced into the expression vectors were confirmed by sequencing at the Interdisciplinary Center for Biotechnology Research (ICBR) (University of Florida, Gainesville, Fla.). All primers are listed in Table 1-1.

TABLE 1-1
List of primers used in building expression vector

| Primer name | Sequence 5′-3′* | Description* |
|---|---|---|
| C-749 | AGT CCT CGA GAA CCA CTT AGT TGT | 3′end of p18 (CTV T36 clones |
| | TTA GCT ATC | nts # 17121-17145) with an |
| | | added XhoI site before nt |
| | | #17121) (downstream of this |
| | | primer there exist within CTV |
| | | genome a PstI site (nts 17208- |
| | | 17213 of CTV T36) used for |
| | | cloning) (F.P.) |
| C-1358 | TTA TGC GGC CGC AGG CCT TGG ACC | 3′end of 3′NTR (nts 19,270- |
| | TAT GTT GGC CCC CCA TAG | 19,293 of CTV T36 clone) |
| | | contain (StuI and NotI sites) |
| | | (R.P.) |
| C-1568 | TAA TCG TAC TTG AGT TCT AAT ATG | 5′end of GFP (nts 1-21) with |
| | GCT AGC AAA GGA GAA GAA | extension into 3′ end of BYV |
| | | CP IR (nts # 13620-13640 |
| | | Genbank Accession # |
| | | AF190581) (F.P.) |
| C-1894 | GCC GCA CTA GTA TTT AAA TCC CGT | 3′end of 3′NTR (nts 19,262- |
| | TTC GTC CTT TAG GGA CTC GTC AGT | 19,293 of CTV T36 clone) |
| | GTA CTG ATATAA GTA CAG ACT GGA | with extensions that include a |
| | CCT ATG TTG GCC CCC CAT AGG GAC | ribozyme of subterranean |
| | AGT G | clover virusoid (underlined) |
| | | (Turpen et al., 1993) and SwaI |
| | | and SpeI restriction sites |
| | | (R.P.) |
| C-1973 | ATG GAT GAG CTC TAC AAA TGA TTG | 5′end of 3′NTR (nts 19021- |
| | AAGTGG ACG GAATAA GTT CC | 19043 of CTV T36 clone) |
| | | with extension into GFP 3′end |
| | | (nts 700-720) (underlined) |
| | | (F.P.) |
| C-1974 | GGA ACT TAT TCC GTC CACTTC AAT | 3′end of GFP (nts 700- |
| | CAT TTG TAG AGCTCA TCC AT | 720) (underlined) with |
| | | extension into 5′end of 3′NTR |
| | | (nts 19021-19043 of CTV T36 |
| | | clone) (R.P.) |
| C-1975 | GCA CGT TGT GCT ATA GTA CGT GCC | GLRaV-2 intergenic region of |
| | ATA ATA GTG AGT GCT AGC AAA | CP (nts 9568-9651 Genbank |
| | GTATAA ACG CTG GTGTTT AGC GCA | Accession number |
| | TAT TAA ATA CTA ACG | DQ286725) (underlined) (F.P.) |
| C-1976 | CAG CTT GCT TCT ACCTGA CAC AGT | BYSV CP intergenic region of |
| | TAA GAA GCG GCATAA ATC GAA GCC | (nts 8516-8616 Genbank |
| | AAA CCCTAA ATT TTG CAA | accesion # U51931) |
| | CTC GAT CAATTG TAA CCT AGA GCG | (underlined) (F.P). |
| | AAGTGC AAT CA | |
| C-1977 | TTT AGC GCA TAT TAA ATA CTA ACG | 5′ end of GFP (nts 1- |
| | ATG GCT AGC AAA GGA GAA GAA | 21) (underlined) with extension |
| | | into the 3′end of GLRaV-2 CP |
| | | intergenic region (nts 9628- |
| | | 9651 Genbank Accession |
| | | number DQ286725) (italics) |
| | | (F.P.) |
| C-1979 | ACT GTG TCA GGT AGA AGC AAG CTG | 3′end of p23 (nts 19,000- |
| | TCA GAT GAA GTG GTGTTC ACG | 19,020 of CTV T36 clone) |
| | | with extension into 5′end of |
| | | BYSV CP IR (nts 8516-8539 |
| | | Genbank accesion # |
| | | U51931) (underlined) (R.P.) |
| C-1982 | TTG GAT TTA GGT GAC ACT ATA GTG | Sp6 promoter (underlined and |
| | GAC CTATGTTGG CCC CCC ATA | Italics) with 3′ end of 3′NTR |
| | | (nts 19271-19293 of CTV T36 |
| | | clone) used to develop dig |
| | | labeled probe (R.P.) |
| C-1983 | GTA ACCTAG AGC GAA GTG CAA TCA | 5′end of GFP (nts 1- |
| | ATG GCT AGC AAA GGA GAA GAA | 23) (underlined) with extension |
| | | into 3′end of BYSV IR of CP |
| | | (nts 8593-8616 Genbank |
| | | Accession # U51931) (italics) |
| | | (F.P.) |
| C-1984 | GCC TAA GCT TAC AAA TAC TCC CCC | 3X active ribosome complementary |
| | ACA ACA GCT TAC AAT ACT CCC CCA | sequence (3XARC-1 nts 1-86 ) |
| | CAC AGC TTA CAA ATA CTC CCC CAC | (Akbergenov et al., 2004) |
| | AAC AGCTTG TCG AC | (F.P.) |
| C-1985 | CTC CGT GAA CAC CACTTC ATC TGA | 5′ end of TEV 5′NTR (nts 1- |
| | AAA TAA CAA ATC TCA ACA CAA | 21 Genbank Accession # |
| | | M11458) (underlined) with |
| | | extension into 3′ end of p23 |
| | | (nts 18997-19020 of CTV T36 |
| | | clone) (F.P.) |
| C-1986 | TTG TGT TGA GAT TTG TTA TTT TCA | 3′end of p23 (nts 18997- |
| | GAT GAA GTG GTG TTC ACG GAG | 19020 of CTV T36 clone) |
| | | with extension into 5′ end of |
| | | TEV 5′NTR (nts 1-21 |
| | | Genbank Accession # |
| | | M11458) (underlined) (R.P.) |
| C-1989 | GGA GTATTT GTA AGCTTA GGC TCA | 3′end of p23 (nts 18997- |
| | GAT GAA GTG GTGTTC ACG GAG | 19020 of CTV T36 clone) |
| | | with extension into 5′end of |
| | | 3XARC-1 (nts 1- |
| | | 21) (underlined) (R.P.) |
| C-1990 | CCC CAC AAC AGCTTG TCG ACA TGG | 5′end of GFP (nts 1- |
| | CTA GCA AAG GAG AAG AAC TTT | 25) (italics) with extension into |
| | | 3′end of 3XARC-1 (nts 66- |
| | | 86) (underlined) (F.P.) |
| C-2007 | CGT GAA CAC CACTTC ATC TGA TTC | BYV 3′end of CPm and the |
| | GAC CTC GGT CGT CTT AGT TAA | intergenic region of CP (nts |
| | | 13547-13570 Genbank |
| | | Accession # |
| | | AF190581) (underlined) with |
| | | extension into p23 3′end (nts |
| | | 19,000-19,020 of CTV T36 |
| | | clone) (F.P.) |
| C-2008 | TTA ACT AAG ACG ACC GAG GTC GAA | 3′end of p23 (nts 19,000- |
| | TCA GAT GAA GTG GTG TTC ACG | 19,020 of CTV T36 clone) |
| | | with extension into the 3′end |
| | | of CPm and CP intergenic |
| | | region of BYV (nts 13,547- |
| | | 13,570 Genbank Accession # |
| | | AF190581) (underlined) (R.P.) |
| C-2009 | GGC GAT CAC GAC AGA GCC GTGTCA | GLRaV-2 3′end of CPm and |
| | ATT GTC GCG GCT AAG AAT GCT GTG | 5′ end of CP intergenic region |
| | GAT CGC AGC GCT TTC ACT GGA GGG | (nts 9454-9590 Genbank |
| | GAG AGA AAA ATA GTT AGT TTG TAT | Accession number |
| | GCCTTA GGA AGG AACTAA GCA CGT | DQ286725) (F.P.) |
| | TGT GCT ATA GTA CGT GC | |
| C-2010 | TGA CAC GGC TCT GTC GTG ATC GCC | 3′end of p23 (nts 19,000- |
| | TCA GAT GAA GTG GTGTTC ACG | 19,020 of CTV T36 clone) |
| | | with extension into the 3′end |
| | | of GLRaV-2 CPm coding |
| | | sequence (nts 9454-9477 |
| | | Genbank Accession # |
| | | DQ286725) (underlined) (R.P.) |
| C-2011 | GCC ACC TAC GTT ATA GGT CTT CAT | 3′end of GFP (nts 697-717) |
| | TTT GTA GAG CTC ATC CAT GCC | (italics) with extension into |
| | | the TEV HC-Pro protease |
| | | recognition sequence (nts |
| | | 2412-2435(genetic code |
| | | redundancy used to eliminate |
| | | duplication Genbank |
| | | Accession # M11458) |
| | | (underlined) (R.P.) |
| C-2012 | AAG ACC TAT AAC GTA GGT GGC ATG | 5′ end of TEV HC-Pro |
| | AAG GCT CAATAT TCG GAT CTA | protease motif (nts 1959-1979 |
| | | Genbank Accession # |
| | | M11458) (italics) with |
| | | extension into the HC-Pro |
| | | recognition sequence (nts |
| | | 2415-2438 genetic code |
| | | redundancy used to eliminate |
| | | duplication Genbank |
| | | Accession # M11458) |
| | | (underlined) (F.P.) |
| C-2013 | ATG AAA ACT TAC AAT GTT GGA GGG | 5′end of GUS (nts 4-21) |
| | ATG TTA CGT CCT GTA GAA ACC | (italics) with extension |
| | | into the TEV HC-Pro |
| | | recognition sequence and 3′ |
| | | end of TEV HC-Pro protease |
| | | motif (nts 2412-2438 Genbank |
| | | Accession # M11458) |
| | | (underlined) (F.P.) |
| C-2014 | GGT TTC TAC AGG ACG TAA CAT CCC | TEV HC-Pro recognition |
| | TCC AAC ATT GTA AGT TTT CAT | sequence (nts 2412-2438 |
| | | Genbank Accession # |
| | | M11458) (underlined) with |
| | | extension into the 5′ end of |
| | | GUS ORF sequence (nts 4- |
| | | 21) (italics) (R.P.) |
| C-2015 | CCG CAG CAG GGA GGC AAA CAA | 5′ end of 3′NTR (nts 19021- |
| | TGA TTG AAGTGG ACG GAA TAA GTT | 19041 of CTV T36 clone) |
| | | with extension into the 3′ end |
| | | of GUS ORF (nts 1789- |
| | | 1812) (underlined) (F.P.) |
| C-2016 | AAC TTA TTC CGT CCA CTT CAA TCA | 3′ end of GUS (nts 1789- |
| | TTG TTT GCCTCC CTG CTG CGG | 1812) (underlined) with |
| | | extension into the 5′end of |
| | | 3′NTR (nts 19021-19041 of |
| | | CTV T36 clone) (R.P.) |
| C-2017 | CTT ACT CTG AAA ATA AAG ATT CTC | 3′end of GFP (nts 697- |
| | TTT GTA GAG CTC ATC CAT GCC | 717) (underlined) with |
| | | extension into the 5′end of |
| | | TEV-NIa protease recognition |
| | | sequence (nts 8499-8519 |
| | | Genbank Accession # |
| | | M11458) and 5′ end of TEV |
| | | NIa protease motif (nts 6270- |
| | | 6272 Genbank Accession # |
| | | M11458) (italics) (R.P.) |
| C-2018 | AAA GAG AAT CTT TAT TTT CAG AGT | 5′ end of TEV NIa protease |
| | AAG GGA CCA CGT GAT TAC AAC | motif (nts 6270-6290 Genbank |
| | | Accession # M11458) |
| | | (underlined) with |
| | | extension into its recognition |
| | | sequence (nts 8499-8519 |
| | | Genbank Accession # |
| | | M11458) and 3′ end of GFP |
| | | (nts 715-717) (italics) (F.P.) |
| C-2019 | CGA TTG GAA GTA TAG GTT TTC TTG | 3′end of TEV NIa motif (nts |
| | CGA GTA CAC CAA TTC ACT CAT | 6961-6980 Genbank Accession # |
| | | M11458) (underlined) with |
| | | extension into NIa recognition |
| | | sequence (nts 8499-8519 |
| | | Genbank Accession # M11458 |
| | | genetic code redundancy used |
| | | to eliminate duplication) |
| | | (R.P.) |
| C-2020 | CAA GAA AAC CTA TAC TTC CAA TCG | 5′end of GUS with extension |
| | ATG TTA CGT CCT GTA GAA ACC | into the TEV NIa recognition |
| | | sequence (nts 8499-8519 |
| | | Genbank Accession # M11458 |
| | | genetic code redundancy used |
| | | to eliminate duplication) |
| | | (underlined) and 3′ end of TEV |
| | | NIa protease motif (nts 6978-6980 |
| | | Genbank Accession # M11458) |
| | | (italics) (F.P.) |
| C-2021 | GTC ACT TTG TTT AGC GTG ACT TAG | 5′end of BYSV CP IR (nts |
| | CAG CTT GCT TCT ACC TGA CAC | 8516-8536 Genbank Accession # |
| | | U51931) (underlined) with |
| | | extension into 3′end of p18 |
| | | (nts 17269-17292 of CTV T36 |
| | | clone) (F.P.) |
| C-2022 | GTG TCA GGT AGA AGC AAG CTG CTA | 3′ end of p18 (nts 17269- |
| | AGT CAC GCT AAA CAA AGT GAC | 17292 of CTV T36 clone) |
| | | with extension into 5′ end |
| | | BYSV CP IR (nts 8516-8536 |
| | | Genbank Accession # |
| | | U51931) (underlined) (R.P.) |
| C-2023 | TTA GTC TCT CCA TCT TGC GTG TAG | 5′end of BYSV CP IR (nts |
| | CAG CTT GCT TCT ACC TGA CAC | 8516-8536 Genbank |
| | | Accession # U51931) |
| | | (underlined) with extension |
| | | into the 3′end of p20 (nts |
| | | 18286-18309 of CTV T36 |
| | | clone) (F.P.) |
| C-2024 | GTG TCA GGT AGA AGC AAG CTG CTA | 3′end of p20 (nts 18286- |
| | CAC GCA AGATGG AGA GAC TAA | 18309 of CTV T36 clone) |
| | | with extension into the 5′ end |
| | | of BYSV CP IR (nts 8516- |
| | | 8536 Genbank Accession # |
| | | U51931) (underlined) (R.P.) |
| C-2025 | ATG GAT GAG CTC TAC AAA TGA-- | 3′end of p13 ORF (nts 17581- |
| | GTT TCA GAA ATT GTC GAATCG CAT | 17604 of CTV T36 clone) |
| | | with extension into the 3′end |
| | | of GFP ORF (nts 700-720) |
| | | (underlined) (F.P.) |
| C-2026 | ATG CGA TTC GAC AAT TTC TGA AAC | 3′end of GFP ORF (nts 700-720) |
| | TCA TTT GTA GAG CTC ATC CAT | (underlined) with |
| | | extension into the 3′end of |
| | | p13 ORF (nts 17581-17604 of |
| | | CTV T36 clone) (R.P.) |
| C-2027 | ATG GAT GAG CTC TAC AAA TGA GTT | 5′end of p23 IR (nts 18,310- |
| | AAT ACG CTT CTC AGA ACG TGT | 18,330 of CTV T36 clone) |
| | | with extension into 3′ end of |
| | | GFP (nts 700-720) |
| | | (underlined) (F.P.) |
| C-2028 | ACA CGT TCT GAG AAG CGT ATT AAC | 3′end of GFP (nts 700-720) |
| | TCA TTT GTA GAG CTC ATC CAT | (underlined) with extension |
| | | into p23 IR (nts 18310-18330 |
| | | of CTV T36 clone) (R.P.) |
| C-2029 | TTT AGC GCATAT TAA ATA CTA ACG | 5′ end of HA TAG (21 nts) in |
| | ATG TAC CCATAC GAT GTT CCA | pHA-CMV carrying bFos (AA |
| | | 118-210)-YC ( AA 155-238) |
| | | (Hu et al., 2002) with |
| | | extension into the GLRaV-2 |
| | | CP IR 3′ end (nts 9628-9651 |
| | | Genbank Accession number |
| | | DQ286725) (underlined) |
| | | (F.P.) |
| C-2030 | TGG AAC ATC GTATGG GTA CAT CGT | 3′ end of CPm GLRaV-2 (nts |
| | TAGTAT TTA ATATGC GCT AAA | 9628-9651 Genbank |
| | | Accession number |
| | | DQ286725) (underlined) with |
| | | extension into 5′ end of HA |
| | | tag (21 nts) in pHA-CMV |
| | | carrying bFos (AA 118-210)- |
| | | YC (AA 155-238) (Hu et al., |
| | | 2002) (R.P.) |
| C-2031 | ACT GTGTCA GGT AGA AGC AAG CTG | 3′end EYFP-YC (AA 232- |
| | TTA CTT GTA CAG CTC GTC CAT | 238) (underlined) (Hu et al., |
| | | 2002) with extension into the |
| | | BYSV CP 5′IR (nts 8516- |
| | | 8539 Genbank Accession # |
| | | U51931) (R.P.) |
| C-2032 | GTA ACCTAG AGC GAA GTG CAATCA | 5′end of FLAG tag (21 nts) |
| | ATG GACTAC AAA GAC GAT GAC | from pFLAG-CMV2 carrying |
| | | bJunN (Hu et al., 2002) with |
| | | extension into the 3′end of |
| | | BYSV CP IR (nts 8593-8616 |
| | | Genbank Accession # |
| | | U51931) (underlined) (F.P.) |
| C-2051 | GTC ACT TTG TTT AGC GTG ACT TAG | 3′end of GLRaV-2 CPm (nts |
| | GGC GAT CAC GAC AGA GCC GTG | 9454-9474 Genbank |
| | | Accession # DQ286725) |
| | | (underlined) with extension |
| | | into 3′end of p18 (nts 17269- |
| | | 17292 of CTV T36 clone) |
| | | (F.P.) |
| C-2052 | CAC GGC TCT GTC GTG ATC GCC CTA | 3′end of p23 (nts 19,000- |
| | AGT CAC GCT AAA CAA AGT GAC | 19,020) with extension into |
| | | the 3′end of GLRaV-2 CPm |
| | | coding sequence (nts 9454- |
| | | 9474 Genbank Accession # |
| | | DQ286725) (underlined) |
| | | (R.P.) |
| C-2053 | GTC ACT TTG TTT AGC GTG ACT TAG | BYV 3′end of CPm and the |
| | TTC GAC CTC GGT CGT CTT AGT | intergenic region of CP (nts |
| | | 13547-13567 Genbank |
| | | Accession # AF190581) |
| | | (underlined) with extension |
| | | into 3′end of p18 (nts 17269- |
| | | 17292 of CTV T36 clone) |
| | | (F.P.) |
| C-2054 | ACT AAG ACG ACC GAG GTC GAA CTA | 3′end of p18 (nts 17269- |
| | AGT CAC GCT AAA CAA AGT GAC | 17292 of T36 CTV clone) |
| | | with extension into BYV |
| | | 3′end of CPm and the |
| | | intergenic region of CP (nts |
| | | 13547-13567 Genbank |
| | | Accession # AF190581) |
| | | (underlined) (R.P.) |
| C-2055 | CAC AAC GTC TAT ATC ATG GCC TAG | 3′end of p13 ORF (nts 17581- |
| | GTT TCA GAA ATT GTC GAA TCG | 17601 of CTV T36 clone) |
| | | (underlined) with extension |
| | | into the 3′end of EYFP- |
| | | YN(AA 147-154) from pFlag- |
| | | CMV2 carrying bJun-YN (Hu |
| | | et al., 2002) |
| C-2056 | CGA TTC GAC AAT TTC TGA AAC CTA | 3′end of EYFP-YN(AA 147- |
| | GGC CAT GAT ATA GAC GTT GTG | 154) (underlined) from pFlag- |
| | | CMV2 carrying bJun-YN (Hu |
| | | et al., 2002) with extension |
| | | into the 3′end of p13 (nts |
| | | 17581-17601 of CTV T36 |
| | | clone) |
| C-2057 | GGC ATG GAC GAG CTG TAC AAGTAA | 3′end EYFP-YC (AA 231- |
| | TTG AAGTGG ACG GAATAA GTT | 238) (underlined) (Hu et al., |
| | | 2002) with extension into |
| | | 5′end of 3′NTR (nts 19021- |
| | | 19041 of CTV T36 clone) |
| C-2058 | AAC TTA TTC CGT CCA CTT CAA TTA | 5′end of 3′NTR (nts 19021- |
| | CTT GTA CAG CTC GTC CAT GCC | 19041 of CTV T36 clone) |
| | | with extension into 3′end |
| | | EYFP-YC (AA 231-238) |
| | | (underlined) (Hu et al., 2002) |
| C-2059 | TCG CTC TTA CCT TGC GAT AAC TAG | BYSV CP 5′IR (nts 8516- |
| | CAG CTT GCT TCT ACCTGA CAC | 8536 Genbank Accession # |
| | | U51931) (underlined) with |
| | | extension into the 3′end of |
| | | p13 (nts 17,662-17,685 of |
| | | CTV T36 clone) (F.P.) |
| C-2063 | GTA ACCTAG AGC GAA GTG CAA TCA | 5′end of GUS ORF (nts 1-21) |
| | ATG TTA CGT CCT GTA GAA ACC | with extension into the 3′ end |
| | | of BYSV CP IR (with |
| | | extension into the 3′end of |
| | | BYSV CP IR (nts 8593-8616 |
| | | Genbank Accession # |
| | | U51931) (underlined) (F.P.) |
| C-2064 | GGT TTC TAC AGG ACG TAA CAT TGA | 3′end of BYSV CP IR (nts |
| | TTG CACTTC GCT CTA GGTTAC AA | 8591-8616 Genbank |
| | | Accession # U51931) |
| | | (underlined) with extension |
| | | into the 5′ end of GUS ORF |
| | | (nts 1-21) (R.P) |
| C-2067 | CCG CAG CAG GGA GGC AAA CAA | 3′end of p13 (nts 17581- |
| | TGA GTT TCA GAA ATT GTC GAATCG | 17601 of CTV T36 clone) |
| | | with extension into the 3′end |
| | | of GUS (nts 1789-1812) |
| | | (underlined) (F.P.) |
| C-2068 | CGA TTC GAC AAT TTC TGA AAC TCA | 3′end of GUS (nts 1789-1812) |
| | TTG TTT GCCTCC CTG CTG CGG | (underlined) with extension |
| | | into the 3′end of p13 (nts |
| | | 17581-17601 of CTV T36 |
| | | clone) |
| C-2069 | GTG TCA GGT AGA AGC AAG CTG CTA | 3′end of p13 (nts 17662- |
| | GTT ATC GCA AGG TAA GAG CGA | 17685 of CTV T36 clone) |
| | | with extension into 5′end of |
| | | BYSV IR CP 5′IR (nts 8516- |
| | | 8536 Genbank Accession # |
| | | U51931) (underlined) (R.P.) |
| C-2070 | ATG GAT GAG CTC TAC AAATGA AGT | 5′IR of p20 (nts 17686-17709 |
| | CTA CTC AGT AGT ACG TCT ATT | of CTV T36 clone) |
| | | (underlined) with extension |
| | | into the 3′end of GFP (nts |
| | | 700-720) (F.P.) |
| C-2071 | AAT AGA CGT ACT ACT GAGTAG ACT | 3′end of GFP (nts 700-720) |
| | TCA TTT GTA GAG CTC ATC CAT | (underlined) with extension |
| | | into the 5′IR of p20 (nts |
| | | 17686-17709 of CTV T36 |
| | | clone) (R.P.) |
| C-2085 | GCG G ATGCAT TATTT GGTTTT ACA | 3′end of p18 (nts 17201- |
| | ACA ACG GTA CGT TTC AAA ATG | 17245 of CTV T36 clone) |
| | | with two point mutations (C- |
| | | A(17205) and G-T(17210)) |
| | | creating NsiI (underlined) site |
| | | to replace the PstI site (F.P.) |
| C-2087 | AAG ACC TAT AAC GTA GGT GGC ATG | 5′ end of TEV HC-Pro |
| | AAG GCT CAA TAT TCG GAT CTA | protease motif (nts 1959-1979 |
| | | Genbank Accession # |
| | | M11458) (underlined) with |
| | | extension into the HC-Pro |
| | | recognition sequence (nts |
| | | 2415-2438 genetic code |
| | | sequence redundancy was |
| | | used to eliminate duplication |
| | | Genbank Accession # M11458 (F.P.) |
| C-2088 | ATG AAA ACT TAC AAT GTT GGA GGG | 5′end of GFP ORF(nts 4-21) |
| | ATG GCT AGC AAA GGA GAA GAA | (underlined) with extension |
| | | into the TEV HC-Pro |
| | | recognition sequence (nts |
| | | 2412-2438 Genbank |
| | | Accession # M11458) (F.P.) |
| C-2089 | TTC TTC TCC TTT GCT AGC CAT CCC | TEV HC-Pro recognition |
| | TCC AAC ATT GTA AGT TTT CAT | sequence (nts 2412-2438 |
| | | Genbank Accession # |
| | | M11458) (underlined) with |
| | | extension into the 5′ end of |
| | | GFP ORF sequence (nts 4-21) |
| | | (R.P.) |
| C-2091 | GAG AAT CTT TAT TTT CAG AGT AAG | 5′ end of TEV NIa protease |
| | GGA CCA CGT GAT TAC AAC C | motif (nts 6270-6291 Genbank |
| | | Accession # M11458) |
| | | (underlined) with extension |
| | | into its recognition sequence |
| | | (nts 8499-8519 Genbank |
| | | Accession # M11458) (F.P.) |
| C-2092 | GAA AAC CTA TACTTC CAATCG ATG | 5′end of GFP ORF (nts 1-23) |
| | GCT AGC AAA GGA GAA GAA CT | (underlined) with extension |
| | | into the TEV- NIa protease |
| | | recognition sequence (nts |
| | | 8499-8519 genetic code |
| | | seqence redundancy used to |
| | | eliminate duplication Genbank |
| | | Accession # M11458) (F.P.) |
| C-2093 | AGT TCT TCT CCT TTG CTA GC CAT | TEV NIa protease recognition |
| | CGA TTG GAA GTA | sequence (nts 8499-8519 |
| | TAG GTT TTC | genetic code sequence |
| | | redundancy used to eliminate |
| | | duplication Genbank |
| | | Accession # M11458) |
| | | (underlined) with extension |
| | | into the GFP ORF sequence |
| | | (nts 1-23) (R.P.) |
| C-2094 | AAG ACCTAT AAC GTA GGT GGC ATG | 5′ end of TEV-NIa protease |
| | AAG GGA CCA CGT GAT TAC AAC | motif sequence nts 6270-6291 |
| | | Genbank Accession # |
| | | M11458) (underlined) with |
| | | extension into the HC-Pro |
| | | recognition sequence (nts |
| | | 2415-2438 genetic code |
| | | sequence redundancy was |
| | | used to eliminate duplication |
| | | Genbank Accession # |
| | | M11458) (F.P.) |
| C-2095 | CCC TCC AAC ATT GTA AGT TTT CAT | 3′end of TEV NIa protease |
| | TTG CGA GTA CAC CAATTC ACT | motif(nts 6959-6981 Genbank |
| | | accession # DQ986288) |
| | | (underlined) with extension |
| | | into the TEV HC-Pro protease |
| | | motif (nts 2415-2438 Genbank |
| | | accession # M11458) (R.P.) |
| C-2096 | GAG AAT CTT TAT TTT CAG AGT AAG | 5′end of TEV HC-Pro |
| | GCT CAATAT TCG GAT CTA AAG | protease motif (nts 1959-1979 |
| | | Genbank Accession # |
| | | M11458) (underlined) with |
| | | extension into the TEV NIa |
| | | protease recognition sequence |
| | | (nts 8499-8519 Genbank |
| | | accession # M11458) (F.P.) |
| C-2097 | CGA TTG GAA GTATAG GTT TTC TTC | 3′end of HC-Pro protease |
| | GGATTC CAA ACCTGA ATG AAC | motif (nts 2388-2411 Genbank |
| | | accession # M11458) |
| | | (underlined) with extension |
| | | into the TEV NIa protease |
| | | recognition sequence (nts |
| | | 8499-8519 Genbank accession |
| | | # M11458) (R.P.) |
| C-2098 | GCC ACCTAC GTT ATA GGT CTT CAT | 3′end of p23(nts 18997-19017 |
| | GAT GAA GTG GTGTTC ACG GAG | of CTV T36 clone) |
| | | (underlined) with extension |
| | | into the 5′end of TEV HC-Pro |
| | | protease recognition sequence |
| | | (nts 2412-2435 (genetic code |
| | | seqence redundancy used to |
| | | eliminate duplication) |
| | | Genbank Accession # |
| | | M11458) (R.P.) |
| C-2099 | ACT CTG AAA ATA AAG ATT CTC GAT | 3′end of p23(nts 18994-19017 |
| | GAA GTG GTGTTC ACG GAG AAC | of CTV T36 clone) |
| | | (underlined) with extension |
| | | into the 5′end of TEV NIa |
| | | protease recognition sequence |
| | | (nts 8499-8519 Genbank |
| | | Accession # M11458) (R.P.) |
| M-804 | CAT TTA CGA ACG ATA GCC ATG GCT | 5′end of GFP (nts 1-20) |
| | AGC AAA GGA GAA GAA | (underlined) with 3′end of |
| | | TEV 5′NTR (nts 126-143 |
| | | Genbank Accession # M11458) |
| | | (F.P.) |

Polymerase Chain Reaction (PCR)

PCR was performed using diluted plasmids (1:50) as templates using Vent DNA polymerase (New England Biolabs, Ipswich, Mass.) according to the manufacturer recommendations.

Agro-Injection/Infiltration

Agro-inoculation of Nicotiana benthamiana was performed according to the procedure developed by Gowda et al., (2005) with minor modifications. Agrobacterium tumefaciens EHA 105 was transformed with the binary plasmid containing CTV, variants (expression vectors) and silencing suppressors (p19 of Tomato bushy stunt virus (Gowda et al., 2005); p24 of GLRaV-2 (Chiba et al., 2007), P1/HC-Pro of Turnip mosaic virus (Kasschau et al., 2003) and p22 of Tomato chlorosis virus (Cañizares et al., 2008) by heat shock method (37° C. for 5 minuntes) and subsequently were grown at 28° C. for 48 hours (hrs) on luria burtani (LB) (Sigma-Aldrich, St Louis, Mo.) plates supplemented with antibiotics (kanamycin (50microgram (μg)/milliliter (ml)) and Rifampicilin ((50 μg/ml)). The colonies (two individual colonies per construct) were grown overnight as seed cultures in LB medium supplemented with antibiotics. On the next day 0.5 ml of the seed culture was used to inoculate 35 ml of LB medium supplemented with antibiotics for overnight growth. The bacterial culture was centrifuged at 6,000 rotation per minute (rpm) and resuspended in 10 milli molar (mM) MgCL₂ and 10 mM MES. The pellet was washed with 10 mM MgCL₂ and 10 mM MES and suspended in induction medium; 10 mM MgCL₂ and 10 mM MES containing acetosyringone at a final concentration of 150 μM. The suspension was incubated in the induction medium for at least 5 hrs before injection into the stem or infiltration into the abaxial (lower) surface of N. benthamiana leaves.

Plant Growth Conditions

N. benthmaiana plants maintained in a growth-room (21° C. with 16 hrs of light in a 24 hr period) were used for agro-injection/agro-infiltration four weeks after tansplanting.

Infection of Citrus Plants

Recombinant virions of CTV for infection of citrus plants were obtained from infiltrated and/or systemic leaves of N. benthamiana. The virions were partially purified and enriched by concentration over a sucrose cushion in a TL 100 or SW41 rotor (Robertson et al., 2005). Virions of constructs expressing two foreign proteins were concentrated two times over a step gradient followed by a cushion gradient in SW28 and SW41 rotors, respectively (Garnsey and Henderson, 1982). Inoculation of citrus plants was carried out by bark flap inoculation into 1-1.5 year old Citrus macrophylla seedlings (Robertson et al., 2005) which were grown in a greenhouse with temperatures ranging between approximately 25-32° C.

Protoplast Preparation, Transfection, RNA Isolation and Northern Blot Analysis

N. benthamiana leaf mesopyhll protoplasts were prepared according to the procedure previously developed by Nava-Castillo et al., (1997). Surface sterilized leaves from three week old N. benthamiana plants were gently slashed on the lower side with a sterile blade and incubated overnight in the dark (16-20 hrs) in 0.7M MMC (0.7M mannitol, 5 mM MES, 10 mM CaCl₂) supplemented with the 1% cellulose (Yakult Honsh, Tokyo, Japan) and 0.5% macerase pectinase enzymes (Calbiochem, La Jolla, Calif.).

Capped in vitro RNA transcripts from NotI or StuI linearized plasmid DNA were generated (Satyanarayana et al., 1999) using Sp6 RNA polymerase (Epicentre Technologies, Wis.) and were transfected into the protoplasts using PEG (poly ethylene glycol) as described by Satyanarayana et al., (1999). Four days after transfection, protoplasts were used for preparation of total RNA for northern blot hybridization analysis and isolation of virions. Protoplasts were pelleted in equal amounts in two 1.5 ml eppendorf tubes. The first tube was flash frozen in liquid nitrogen and stored at −80° C. for isolation of virions to subsequently inoculate a new batch of protoplasts to amplify virions (Satyanarayana et al., 2000). The second tube was used for RNA isolation by the buffard buffer disruption of protoplasts followed by phenol: chloroform: isoamyl alcohol (25:24:1) extraction and ethanol precipitation as previously described by Navas-Castillo et al., (1997) and Robertson et al., (2005). Total RNA was resuspended in 20 μl DNAse/RNAase free water and used in Northern blot hybridization analysis as previously described by Lewandowski and Dawson (1998). In brief, isolated RNA was heat denatured in denaturing buffer (8.6% formaldehyde, 67% formamide in 1×MOPS (5 mM sodium acetate, 1 mM EDTA, 0.02M MOPS pH=7.0) separated in a 0.9% agarose gel in 1×MOPS containing 1.9% formaldehyde, and transferred onto a nylon membrane (Boehringer Mannheim, Germany) by electroblotting. Pre-hybridization (at least 1 hr) and hybridization (overnight) were carried out in a hybridization oven (Sigma-Aldrich, St. Louis, Mo.) at 68° C. A 900 nts digoxigenin labeled RNA probe corresponding to the 3′ end of the CTV genome (plus strand specific CTV RNA probe) (Satyanarayana et al., 1999) was used for hybridization except when the insertion of the foreign genetic material was behind p23 in which case a digoxigenin labeled RNA probe was produced from PCR amplified DNA (reverse primer contain 3′NTR of CTV and SP6 phage promoter (C-1982) according to the manufacturer recommendation (Boehringer Mannheim, Germany) that is complimentary to the sequence inserted behind p23 in addition to the 3′NTR sequence of CTV.

Western Blots

After powdering the plant tissue in liquid nitrogen via grinding in a mortar and pestle, laemmli buffer (50 mM Tris-Cl, pH 6.8, 2.5% 2-mercaptoethanol, 2% SDS, 0.1% bromophenol blue, 10% glycerol) was added (1000 per 100mg tissue). The sample was transferred to a 1.5 ml centrifuge tube and boiled in a water bath for 3 minutes followed by centrifugation at maximum speed for 2 minutes. The supernatant was transferred to a new tube and stored at −20° C. until further use. The electrophoresis was carried out in a 12% SDS-Polyacrylamide gel (Bio-Rad, Hercules, Calif.) followed by two hours of semi-dry blotting to transfer the protein onto a nitrocellulose membrane (Bio-Rad, Hercules, Calif.). The membrane was blocked for 1 hr at room temperature followed by incubation with the primary antibody of either CP (1:5000), GFP (1:100) (Clontech Laboratories, Palo Alto, Calif.) or GUS (1:1000) (Molecular probes, Eugene, Oreg.) for an hour followed incubation for 1 hr in horseradish peroxidase conjugated donkey anti-rabbit secondary antibody (1:10,000) (Amersham, Buckinghamshire, United Kingdom). Finally, the chemiluminescent system for western blot (Amersham, Buckinghamshire, United Kingdom) development on an X-ray film (Kodak, Rochester, N.Y.) was used according to the manufacturer recommendations.

Plant and Protoplast Photos

Plant pictures under UV or white light were taken with a Canon Camera (Canon EOS Digital Rebel XTi 400D, Lake Success, N.Y.). Close up fluorescent pictures of plant parts or protoplast were taken using a fluorescent dissecting microscope (Zeiss Stemi SV 11 UV-fluorescence dissecting microscope, Carl Zeiss Jena, GmbH., Jena, Germany). High resolution protoplast pictures were taken using a confocal scanning microscope (Leica TCS SL, Leica Microsystems, Inc., Exton, Pa.).

Enzyme Linked Immunosorbent Assay (ELISA)

Double antibody sandwiched ELISA was used according to the procedure developed by Garnsey and Cambra (1991). A rabbit polyclonal antibody (1 μg/ml) was used for coating the ELISA plate. The plant tissue sample was diluted at a 1:20 in PBS-T (phosphate buffer saline-1% Tween 20) extraction buffer. The detection antibody used was Mab ECTV 172 (1:100K dilution).

GUS Assay

Citrus bark pieces or systemic leaves from Agro-inoculated N. benthamiana plants that were surface sterilized in alcohol (70% ethanol) followed by Sodium hypo chloride (10% solution) and washing three times in sterile distilled water before staining for GUS. The samples were incubated overnight in an EDTA-phosphate buffer (0.1M Na₂HPO₄, 1 mM Na₂EDTA) containing 1 mg/ml X-gluc (cyclohexylammounium salt: Gold Biotechnology, St Louis, Mo.). Fixing of the tissue was done in 95% ethanol: glacial acetic acid solution (3:1).

### Discussion Related to CTV Vector Examples 1-8

In this work, CTV constructs that are extraordinarily permissive in allowing insertion of foreign sequences at different places in the 3′ portion of the genome are disclosed. Numerous different potential vector constructs to express foreign genes via additional subgenomic RNAs, di-cistronic mRNAs, or protease processing of fusion proteins were created and examined. Remarkably, most of these constructs functioned as vectors. Additionally, that the CTV constructs disclosed herein are capable of simultaneously producing large amounts of multiple foreign proteins or peptides.

The ultimate goal was to develop high expressing and stable vectors for the natural CTV host, citrus. Thus, virions were concentrated from N. benthamiana plants infected with 12 different constructs that spread and expressed moderate to high levels of the foreign protein(s) and used to inoculate citrus. C macrophylla plants became positive for infection between 6-60 weeks after inoculation depending on the insert length in the virus and the amount of virions concentrated from the N. benthamiana leaves that were used for inoculation. Most of the constructs that infected citrus produced moderate levels of the reporter gene/s.

Several approaches were examined for expression of foreign genes from CTV. The first approach was the “add-a-gene” strategy that involved the addition or duplication of a controller element and an additional ORF, which resulted in an additional subgenomic RNA. The “add-a-gene” approach was developed initially in TMV via duplicating the CP subgenomic promoter controlling a foreign gene (Dawson et al., 1989; Donson et al., 1991; Shivprasad et al., 1999). An advantage of this strategy is that it expresses the exact protein with no additional amino acids added to the N or/and C terminus which could affect its biological activity, at relatively high levels. However, there are limitations of this strategy that should be considered. Duplication of the controller element can lead to homologous recombination resulting in the loss of the gene of interest (Chapman et al., 1992; Dawson et al., 1989). Although this made the TMV insert unstable, it appeared to have little effect on the stability in CTV (Folimonov et al., 2007). The use of a heterologous controller element from related viruses stabilized the TMV insertions. However, heterologous controller elements usually are differentially recognized by the replicase complex of the virus (Folimonov et al., 2007; Shivprasad et al., 1999). This observation can be utilized to regulate the levels of desired gene expression (Shivprasad et al., 1999). An important consideration is that there can be competition between the different subgenomic RNAs of a virus. With TMV, the extra gene competed with the coat protein gene and the movement gene. There appeared to be a maximal capacity for production of subgenomic RNAs that was divided among the three RNAs. Manipulations that resulted in increases in one resulted in decreases in the others. One solution was to reduce coat protein production to allow optimal foreign gene and movement gene expression (Shivprasad et al., 1999; Girdishivelli et al., 2000). Yet, CTV subgenomic mRNAs appeared to be much less competitive (Folimonov et al., 2007; Ayllón et al., 2003).

In previous work, a CTV vector was created that expressed an extra gene between the CP and CPm genes that was an effective and stable vector in citrus trees. The foreign gene was in position 6 from the 3′ terminus (Folimonov et al., 2007). The position of the extra gene was chosen arbitrarily. Vector design was continued in an attempt to define the limits of manipulation of the CTV genome in producing extra proteins or peptides. The virus expresses its ten 3′ genes via sg mRNAs (Hilf et al., 1995). One rule of CTV gene expression is that genes nearer the 3′ terminus are transcribed higher than internal genes. For example, transcription of the p33 gene, which is at position 10 from the 3′ terminus, is very low in its native position, but transcription became very high when the p33 gene was moved near the 3′ terminus (Satyanarayana et al., 1999). Thus, expression of foreign genes from positions nearer the 3′ terminus might result in higher levels than from the position 6 arbitrarily chosen in the first vector (Folimonov et al., 2007). Yet, based on results from other viruses, only certain positions within the viral genome are likely to tolerate extra gene insertions. For example, with TMV or Alfalfa mosaic virus the location between CP and 3′NTR did not accommodate an insert (Dawson et al., 1989; Lehto and Dawson, 1990; Sanchez-Navarro et al., 2001). Remarkably, almost all of the constructs with insertions in CTV within the p13 deletion, between p13 and p20, and between p23 and the 3′ NTR were viable. In contrast, it was found that the only position the virus did not tolerate insertions was between the p20 and p23 genes. It is possible that these insertions interfered with the transcription of either of the adjacent genes.

Another strategy to express foreign genes in a viral vector consists of in-frame fusion of an ORF of interest to a viral ORF at either the N or C terminus. The two proteins can be released by engineering a protease and processing sites between the two proteins (Dolja et al., 1997; Gopinath et al., 2000). It was first adapted in the potyviridae, tobacco etch virus (Dolja et al., 1992). The major advantage of polyprotein fusion strategy is that the foreign protein is expressed in 1:1 ratio with the viral protein. A major limitation is that this process adds extra amino acids at the N and/or C termini of both proteins, which may affect their biological activities.

A series of constructs utilizing the HC-Pro or NIa proteases from potyviruses to enable post translational processing of the engineered polyprotein to release free GFP, protease, and the p23 protein were created. These vectors were able to systemically infect N. benthamiana. The systemic movement of these constructs was slower than the expression vector constructs containing only the GFP ORF as an extra gene. The slower systemic movement and the lower levels of GFP expression in the systemic leaves partially could be attributed to the extra C-terminal amino acids of p23 reduced its activity in RNA silencing suppression or amplification of viral RNAs or the protease processing delayed its activity. Although these constructs did not produce the maximal levels of foreign protein, they were viable vectors expressing substantial amounts of GFP.

Upon identifying the locations within the CTV genome that could accommodate foreign gene inserts, strategies were designed to construct viral vectors that express multiple genes. The first strategy depended on the use of a single controller element driving the transcription of a polypeptide gene. The fusion gene that consisted of GFP/Pro/GUS, ranged in size from 3127 nts to 3480 nts. Other strategies utilized two extra CEs to produce two extra sg RNAs simultaneously. This strategy gave the flexibility to insert the two genes in tandem in the same location or in two different locations. Both strategies worked.

Heterologous protein expression in whole plant is usually accomplished by development of transgenic plants by insertion of foreign DNA into the plastid or nuclear genome. Plastid transformation has been successful for only a few annual crops. Time and success of nuclear transformation varies among the different crops. Certain plants are more recalcitrant to transformation and subsequent regeneration than others. There are other disadvantages, particularly in perennial crops. For example, citrus has a long juvenile stage after regeneration that prolongs the time necessary to evaluate the horticultural characteristics and delays the time to commercial use. Another major disadvantage is that transformation is limited to the next generation of plants.

A series of different CTV vectors was developed, each with different characteristics that are more effective under specific conditions. For example, with the “add-a-gene” vectors, the expression of a small gene occurs when placed 3′ of the p23 gene in CTV for maximal expression. A medium gene could be more efficiently expressed from within the p13 area. A large gene probably would be better accommodated as an insertion between CP and CPm where it would disrupt the viral subgenomic RNAs less and result in better systemic invasion of the plant. For expression of smaller proteins, peptides, or RNAs to target RNA silencing, it is possible that the virus could accommodate 3 or 4 different genes. Different combinations of extra sg RNAs and protease processing can be chosen. Although two foreign proteins have been produced from other viruses, CTV is unique in usefulness because of its stability. The original vector has been continuously producing GFP for 8 years.

The uses of the CTV based expression vector have evolved since its inception. It was initially developed as a laboratory tool for citrus improvement. The vector was designed to express potential genes for transformation of citrus. Results of the effect of the heterologous gene in citrus, particularly if the effect was expected in mature tissue or fruit, could be obtained by the virus years before results would come from direct transformation. However, conditions and needs of the citrus industry have changed due to the invasion of a new bacterial disease referred to as Huanglongbing (HLB). This disease has spread so rapidly and is so damaging that the survival of the citrus industry is threatened. Initially, the CTV vector was used to identify antimicrobial peptides with activity against the HLB bacterium for transformation into citrus. However, the disease is spreading so rapidly that transgenic plants may not be available in time to save the industry. Due to the remarkable stability, the CTV vector now is being considered for use in the field to protect citrus trees and to treat infected trees until resistant transgenic plants become available. The CTV vector as a tool in the field to fight an invading disease of citrus is only one example of what viral vectors can do for agriculture. The possibilities are many for very stable vectors like those of CTV and perennial crops, particularly trees. Many trees are productive for 100 years or more. During the lifespan of the trees technologies changes and disease and pest pressures change. To improve trees by traditional transformation methods requires removing all of the present trees from the field and replanting. The use of a viral vector could add new genes to the existing trees.

### CTV VECTOR EXAMPLES

### Example 1

### Systems Used to Examine CTV-Based Expression Vectors

CTV-based expression vectors were examined in three systems, N. benthamiana mesophyll protoplasts as well as whole plants of N. benthaminia and Citrus macropylla. The full-length cDNA clone of CTV (pCTV9R) and a mutant with most of the p33 gene deleted (pCTV9RΔp33), which has a PstI restriction site removed making cloning easier and still retaining the ability to infect most citrus varieties (Tatineni et al., 2008), was used for building constructs to infect whole plants. Relatively quick assays were done in N. benthamiana protoplasts, which require constructs to be built in the SP6 transcription plasmid (Satyanarayana et al., 1999). A mini-replicon pCTVΔCla 333R (Gowda et al., 2001), with most of the 3′ genes removed, was convenient to use in protoplasts. The ultimate goal to obtain citrus trees infected with the different CTV expression vectors was much more difficult and time consuming. So far, agro-inoculate citrus trees has proven difficult. Thus, to avoid this difficulty virions are amplified and concentrated for inoculation of citrus trees by stem-slashing or bark-flap inoculation (Robertson et al., 2005; Satyanarayana et al., 2001). N. benthamiana protoplasts can be inoculated with in vitro produced transcripts of recombinant CTV constructs and the virus amplified by successively passaging virions in crude sap through a series of protoplasts (Folimonov et al., 2007; Satyanarayana et al., 2001; Tatineni et al., 2008). Also, recombinant CTV can be amplified in N. benthamiana plants after agro-inoculation (Gowda et al., 2005). The virus can infect mesophyll cells of agro-inoculated areas of leaves, but as the virus moves systemically into upper non-inoculated leaves, it is limited to vascular tissues and usually induces vein clearing and later vein necrosis. All of the vector constructs were examined during systemic infection of N. benthamiana plants. Since CTV virions do not resuspend after centrifugation to a pellet, virions have to be concentrated by centrifugation through a sucrose step gradient (Garnsey et al., 1977; Robertson et al., 2005). After inoculation, the tops of citrus plants were removed, and viral systemic infections were monitored in new growth after 2-3 months. Once trees were infected, inoculum (buds, leaf pieces, or shoots) from the first infected plants was then used to propagate new plants for experimentation. The whole process takes approximately one year.

### Example 2

### Addition of an Extra Gene at Different Locations Within the CTV Genome

Insertions at the p13 Gene Site

The effective CTV vector developed previously (Folimonov et al., 2007) has the additional gene inserted between the two coat protein genes, positioning the foreign gene as the sixth gene from the 3′ terminus. Yet, the most highly expressed genes of CTV tend to be closer to the 3′ terminus. Thus, it appeared that positioning an inserted gene closer to the 3′ terminus could result in higher levels of expression. P13, the third gene from the 3′ terminus, is a relatively highly expressed gene that is not necessary for the infection of most of the CTV host range (Tatineni et al., 2008; Tatineni et al., in preparation). Yet, replacement of the p13 ORF with the GFP ORF was not successful in previous attempts (Folimonov et al., 2007). There were possible reasons for the failure. The previous construct was designed with the assumption that translation initiated at the first start codon, but the p13 ORF has a second in-frame AUG. Translation might normally start at the second AUG. However, fusion of the GFP ORF behind the second in frame AUG also did not express the reporter gene (Gowda et al., unpublished result). A second possibility is that the p13 controller element (CE) might extend into the p13 ORF or that ribosome recruitment is directed from within the ORF. The p13 CE and ORF were deleted and a new ORF was inserted behind a heterologous CE in the p13 position. The GFP ORF controlled by the CP-CE from BYSV (101 nts from 8516-8616 accession #U51931), GLRaV-2 (198 nts from 9454-9651 accession #DQ286725) or BYV were engineered into pCTV9RΔp33as a replacement for nts 17293-17581 (CTV33-Δ13-BY-GFP-57, CTV33-Δ13-G-GFP-65, CTV33-Δ13-B-GFP-66 respectively) (FIG. 1A). RNA transcripts were used to inoculate a series of protoplasts to determine whether the constructs could replicate and whether virions formed sufficiently for passage in crude sap to a new batch of protoplasts. The fluorescence of infected protoplasts (data not presented) and northern blot hybridization analysis demonstrated the successive passage of the expression vectors through the protoplast transfers (FIG. 1B). Furthermore, the level of the GFP mRNA was similar to that of CP. Vectors sequences CTV33-Δ13-BY-GFP-57, CTV33-Δ13-G-GFP-65 and CTV33-Δ13-B-GFP-66 then were transferred into the Agrobacterium binary plasmid for agro-inoculation of N. benthamiana plants. All three vectors infected and moved systemically in vascular tissue of the N. benthamiana plants as indicated by fluorescence in leaves, buds, flowers and corolla (FIG. 1C), vein clearing phenotype in early stages, as well as confirmed by ELISA (Data not presented).

CTV33-Δ13-G-GFP-65 and CTV33-Δ13-B-GFP-66 were amplified and used to inoculate Citrus macrophylla plants. The initially infected plants exhibited bright fluorescence in vascular tissue (FIG. 1D). Fluorescence continued in these plants 2 years after inoculation.

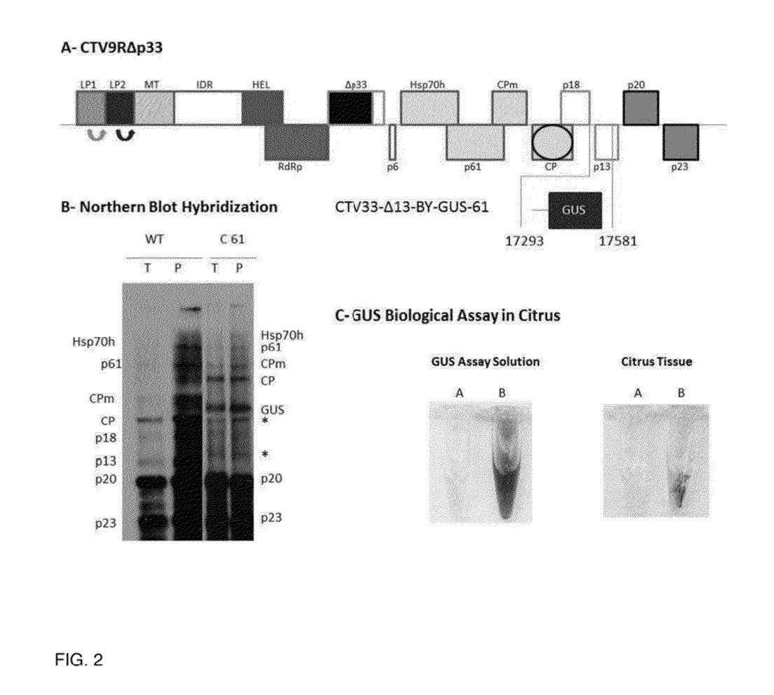

The GFP ORF (720 nts) was replaced with the GUS ORF (1812 nts) in the same position to examine the expression of a larger foreign gene. The BYSV CP-CE was selected to drive the GUS ORF in expression vector CTV33-Δ13-BY-GUS-61 (FIG. 2A). RNA transcripts of this construct were transfected into protoplast where the virus replicated and passaged efficiently from one protoplast batch to another as indicated by northern blot hybridization analysis (FIG. 2B). In addition, it revealed that the level of accumulation of GUS mRNA was identical to the CP mRNA, and the CP and CPm mRNAs of vector were similar to that of the wild type virus. Agro-inoculation of N. benthamiana plants revealed that the construct infected and spread throughout the vascular tissue of the plants based on GUS staining and confirmed by ELISA (Data not presented) and the vein clearing phenotype.

Virions isolated from infiltrated leaves of N. benthamiana plants of CTV33-Δ13-BY-GUS-61 infected Citrus macrophylla plants as confirmed by ELISA (Data not presented) and the bioactivity of the GUS protein (FIG. 2C). The GUS gene was still biologically active in citrus 1.5 year after inoculation.

Technically, the above constructs replaced a gene (p13) rather than added an extra gene. To examine a vector with an extra gene between p13 and p20, the CP-CE of BYSV controlling the GFP ORF was inserted between nts 17685-17686 to yield CTV33-13-BY-GFP-69 (FIG. 3A). This vector should produce an extra subgenomic RNA between the subgenomic RNAs of p13 and p20. Vector CTV33-13-BY-GFP-69 was examined in N. benthamiana protoplasts and plants. In the protoplast system, CTV33-13-BY-GFP-69 replicated efficiently and was successfully passaged from one protoplast batch to another demonstrating efficient replication and virion formation as indicated by fluorescence (Data not presented) and northern blot hybridization analysis (FIG. 3B). The foreign mRNA accumulated at a relatively high level but the CP mRNA was reduced. Similar to the replacement of p13 constructs, agro-inoculation of the expression vector CTV33-13-BY-GFP-69 into N. benthamiana plants enabled the new vector to infect and spread throughout the vascular tissue (FIG. 3C).

Construct CTV33-13-BY-GFP-69 infected C. macrophylla plants as indicated by strong fluorescence throughout the vascular tissue (FIG. 3C) and confirmed by ELISA (Data not presented). The plants were still fluorescencing 2 years after inoculation.

Insertion Between p20 and p23

To examine expression of a foreign gene closer to the 3′ NTR of CTV, an extra gene was inserted between the p20 and p23 genes (nts 18312-18313). The BYV or BYSV CP-CE was used to drive the GFP mRNA in two vectors based on T36 CTV9RΔp33(CTV33-20-B-GFP-49 and CTV33-20-BY-GFP-58) (FIG. 3-4A). The new vectors produced an extra sgRNA mRNA between the p20 and p23 sgRNAs (FIG. 4B). However, the accumulation of the p20 sg mRNA was substantially reduced. Both vectors replicated and were passaged in protoplasts, but the protoplast passage was reduced as demonstrated by reduced numbers of cells with GFP fluorescence and northern blot hybridization (FIGS. 4B & C). When both CTV33-20-B-GFP-49 or CTV33-20-BY-GFP-58 vectors were infiltrated into N. benthamiana leaves for transient expression, the vectors replicated and produced abundant amounts of GFP as indicated by fluorescence (Data not presented) and western blot analysis (FIG. 4D).However, when agro-inoculated into N. benthamiana plants, the constructs replicated but movement into upper non-inoculated leaves was random and often unsuccessful. Since systemic infection of N. benthamiana plants was marginal, no attempt was made to inoculate citrus.

Insertion Between p23 and 3′NTR

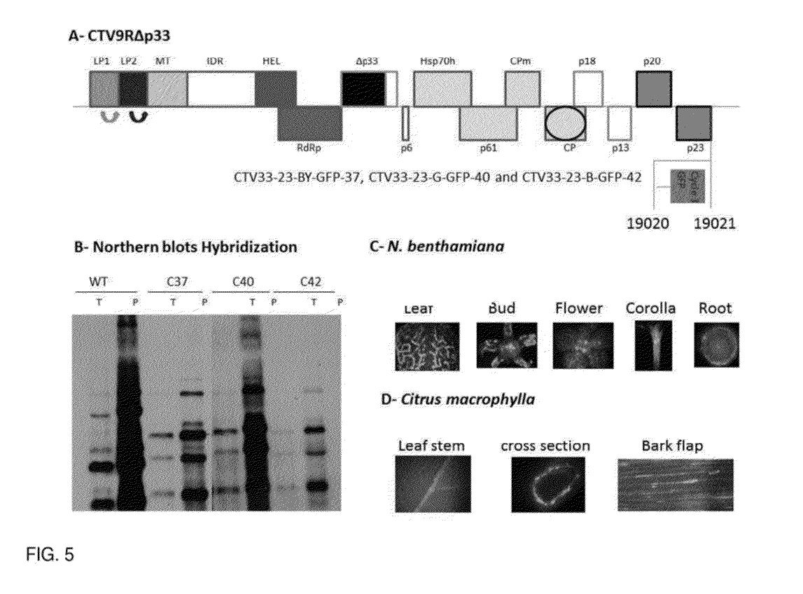

The next position to be examined was to make the inserted gene the 3′-most gene. Since CTV gene expression tends to be highest for genes positions nearer the 3′ terminus, this position could be expected to result in the highest level of expression of a foreign gene (Navas-Castillo et al., 1997; Hilf et al., 1995). Although the 3′ NTR has been analyzed (Satyanarayana et al., 2002a), it was not known what effect an extra gene in this area would have on the efficiency of replication. The insertion of an extra gene between the CP gene and the 3′NTR in Tobacco mosaic virus (TMV) and Alfalfa mosaic virus (AMV) failed to produce viable vectors (Dawson et al., 1989; Sánchez-Navarro et al., 2001). The CP-CE of BYSV, GLRaV-2 or BYV in front of the GFP ORF was inserted between nucleotides 19020 and 19021 creating vectors CTV33-23-BY-GFP-37, CTV33-23-G-GFP-40 and CTV33-23-B-GFP-42, respectively (FIG. 5A). All of the constructs when transfected into the protoplast replicated and were passaged efficiently as indicated by northern blot hybridization analysis (FIG. 5B) and GFP fluorescence (Data not presented). The GFP mRNA was the highest accumulating mRNA, with only slight decreases to the other mRNAs compared to that of the wild type virus (FIG. 5B). Furthermore, the constructs with a GFP insertion 3′ of the p23 ORF had the highest accumulation of the foreign gene mRNA among the constructs examined. CTV33-23-BY-GFP-37, CTV33-23-G-GFP-40 and CTV33-23-B-GFP-42 constructs were agro-inoculated into N. benthamiana plants. The infections spread systemically throughout the vascular tissue as demonstrated by the fluorescence (FIG. 5C), phenotype (vein clearing followed by necrosis), and ELISA (Data not presented). The fluorescence in the vascular tissue of N. benthamiana plants was extremely bright and continued for the life of the infected plants (FIG. 5C)

Construct CTV33-23-BY-GFP-37 was amplified by passage through 12 protoplast sets before citrus inoculation. C macrophylla plants that were bark-flap inoculated with the concentrated virions became infected. The infection of citrus was confirmed by fluorescence of GFP (FIG. 3-5D) and ELISA (Data not presented). Inoculation of citrus with constructs CTV33-23-G-GFP-40 was done via amplification in agro-inoculated N. benthamiana plants. The infection rate was in 1 of 4 C. macrophylla plants as indicated by fluorescence (FIG. 5D) and confirmed by ELISA (Data not presented). Similar to N. benthamiana, citrus plants expressed bright fluorescence in the vascular tissue 12 weeks after inoculation and were still fluorescing 2.5 years later (FIG. 5D).

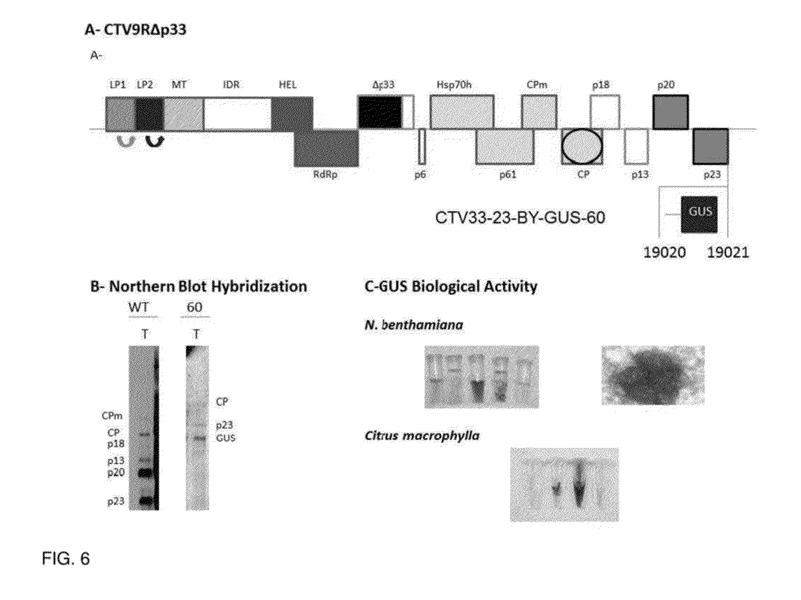

To examine the ability of the vector to express a larger gene at this position, the GUS ORF behind the BYSV CP-CE was inserted 3′ of the p23 gene resulting in construct CTV33-23-BY-GUS-60 (FIG. 6A). The construct replicated in successfully transfected protoplasts. However, the accumulation levels of all the CTV subgenomic RNAs were decreased profoundly compared to the wild type virus as demonstrated by northern blot hybridization analysis (FIG. 6B). Also, the CTV33-23-BY-GUS-60 construct passaged poorly in protoplasts (Data not presented). Yet, after agro-inoculation of N. benthamiana plants, the vector replicated and moved systemically as demonstrated by the systemic symptoms (vein clearing followed by necrosis), ELISA (Data not presented) and GUS assays. The activity of GUS in the N. benthamiana plants was continuously produced in old and new leaves until the death of the plant (FIG. 7C). Similar to CTV33-Δ13-BY-GUS-61, the location between p23 and 3′NTR was able to accommodate moderately to long genes albeit with a differential effect on sg RNA levels of upstream genes (FIG. 5B & FIG. 6B)

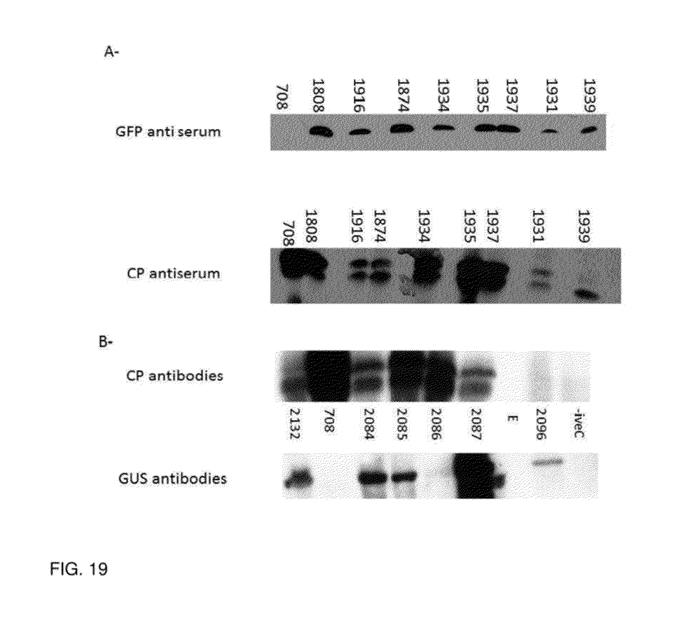

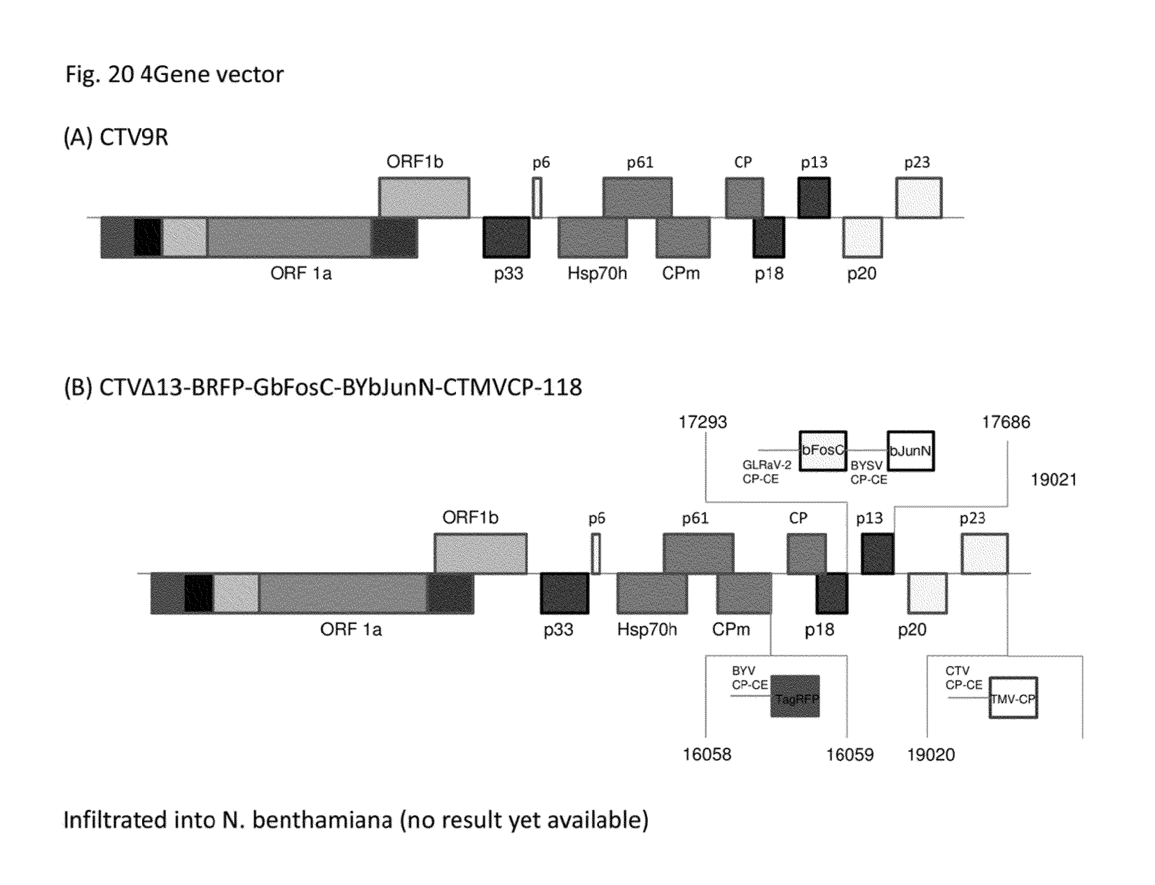

Concentrated virions from Construct CTV33-23-GUS-60 were used to inoculate C. macropyhlla plants, which became infected as confirmed by ELISA (Data not presented) and activity of the GUS gene (FIG. 6C). Furthermore, GUS activity and western blot analysis revealed the presence of the GUS gene in citrus 1.3 years after inoculation (FIG. 6C, FIG. 19).

### Example 3

### Production of an Extra Polypeptide Without Producing an Extra Subgenomic mRNA

Internal Ribosome Entry Site Strategy (IRES)

The Tobacco Etch Virus (TEV) IRES

The 5′NTR of TEV mediates cap independent translation of the viral mRNA. Studies on the 5′NTR of TEV demonstrate its ability to initiate translation at an internal ORF in a bi-cistronic mRNA (Gallie, 2001; Niepel and Gallie, 1999). The 5′NTR of TEV (nts 2-144 Genbank accession #DQ986288) was inserted into a CTV mini-replicon behind the p23 ORF (between nts 19020-19021) followed by the GFP ORF (CTVp333R-23-ITEV-GFP) (FIG. 7A) to examine whether a bicistronic subgenomic mRNA would work with this virus. Although northern blot hybridization analysis demonstrated that the mini-replicon replicated and produced abundant amounts of the bicistronic mRNA in transfected N. benthamiana protoplasts (FIG. 7C), GFP fluorescence was not observed, suggesting a lack of translation of the second ORF in the bicistronic mRNA. The 5′NTR TEV IRES construct in full length CTV was examined in N. benthamiana protoplasts and plants. Construct CTV33-23-ITEV-GFP-41 was passaged efficiently from protoplast to the next protoplast sets (FIG. 7B), indicating the good replication and formation of virions, but no fluorescing protoplasts were observed demonstrating that this IRES did not work well in CTV (data not presented). This construct infected and moved systemically in N. benthamiana plants based on the systemic symptoms of vein clearing followed by necrosis and ELISA (Data not presented), but no GFP fluorescence was observed under UV light (Data not presented).

Active Ribosome Complementary Sequence (ARC) IRES

Insertion of an IRES consensus sequence obtained from analysis of host and viral mRNAs (the engineered 3xARC-1 (86 nts) IRES (Akbergenov et al., 2004)) was next examined for activity in CTV. This IRES was fused behind the p23 ORF (nts 19020-19021) in both the CTV mini-replicon (CTVp333R-23-I3XARC-GFP) and Δp33CTV9R (CTV33-23-I3XARC-GFP-43) as described above (FIG. 7A). However, after infection of protoplasts and plants, no GFP fluorescence was observed even though the virus replicated well in both (FIGS. 7B&C)

Poly-Peptide Fusion

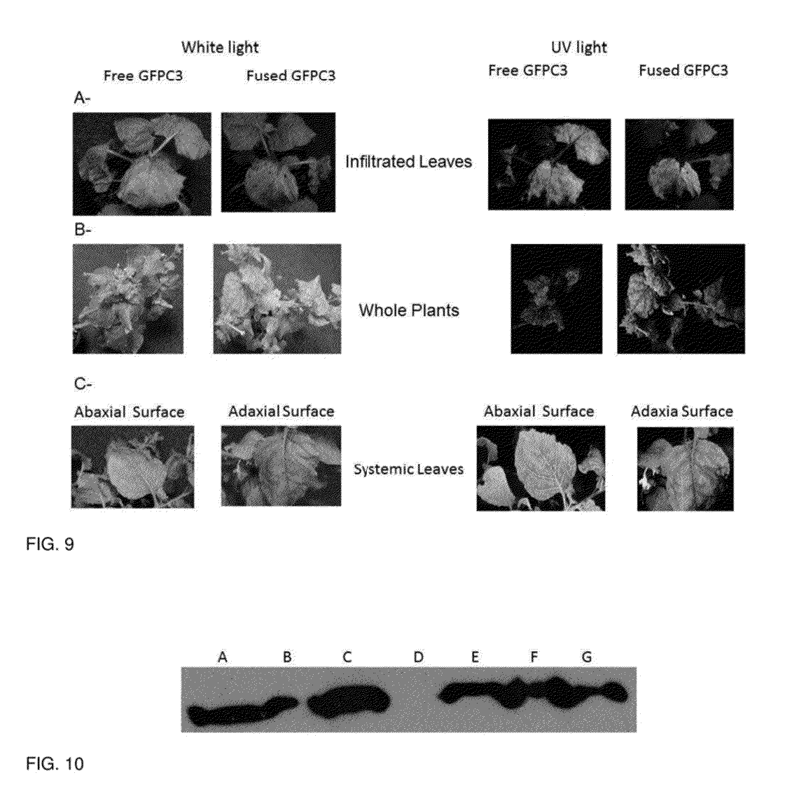

P23, the highest expressed gene of CTV, is a multifunctional protein that is essential for citrus infection. P23 is a silencing suppressor and controls plus to minus RNA ratio in infected cells via an RNA binding domain constituted of positive charged amino acid residues and Zn finger domain present between amino acid 50-86 (Lopez et al., 2000; Satyanarayana et al., 2002b; Lu et al., 2004). In order to create a gene fusion the HC-Pro or NIa protease motifs of TEV were selected to be fused at the C-terminus of p23 (between nts 19017 and 19018) (FIG. 8). The protease recognition sequence of the HC-Pro and NIa was duplicated between p23 and the protease and between the protease and GFP creating vectors CTV33-23-HC-GFP-72 and CTV33-23-NIa-GFP-73, respectively (FIG. 8). The processing of the protease motif from p23 should release the p23 with 7 extra amino acids at its C-terminus in the case of HC-Pro and 6 amino acids in the case of NIa. The GFP protein should have two extra and one extra amino acid after being cleaved from HC-Pro and NIa, respectively. The recognition sequences were switched between HC-Pro and NIa creating vectors CTV33-23-HCØ-GFP-74 and CTV33-23-NIaØ-GFP-75 as controls that are unable to be cleaved (FIG. 8). All the polypeptide fusion vectors were created in CTV binary vectors for infection of plants because in protoplast it was shown that p23 fusion did not affect the ability to replicate and pass between protoplast sets (Tatineni and Dawson, unpublished result). In N. benthamiana infiltrated leaves, all constructs fluoresced similarly to each other and to the free GFP constructs behind p23 (FIG. 9A). Furthermore, western immune-blot analysis from infiltrated leaves indicated a near-perfect processing of the reporter gene from the polypeptide fusion (FIG. 10). The GFP protein did not localize to the nucleus unlike the fusion to p23 without a protease processing releasing the reporter gene. Upon agro-inoculation of plants, only constructs with the protease and its homologous processing sites were able to move systemically into upper non-inoculated leaves. The fluorescence in upper non-inoculated leaves was weaker than those for the expression vectors CTV33-23-BY-GFP-37, CTV33-23-G-GFP-40 and CTV33-23-B-GFP-42 carrying GFP under its own controller element (FIG. 9B). Furthermore, it was easier to visualize fluorescence on the abaxial rather than the adaxial leaf surface (FIG. 9C). Upon inoculation of citrus with construct CTV33-23-HC-GFP-72, one plant became positive with relatively low ELISA value compared to others (Data not presented). The reporter gene activity was not detected.

### Example 4

### Production of More Than One Extra Foreign Protein from CTV Vectors

Use of Single Controller Elements to Express Multiple Proteins

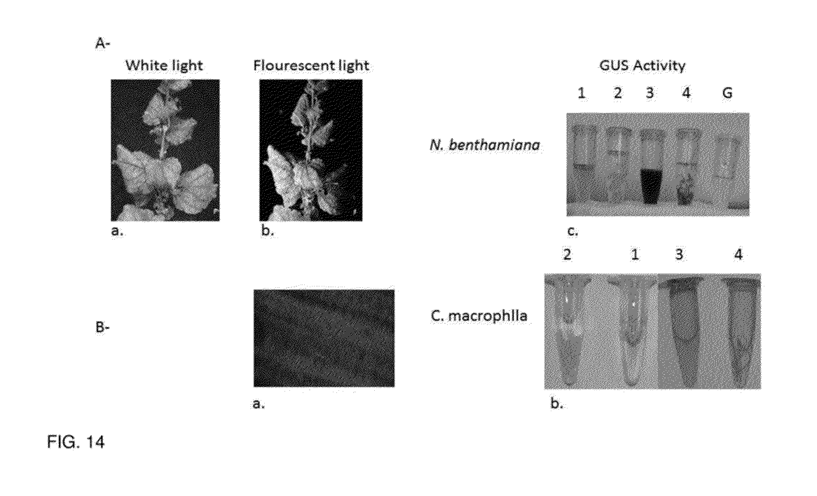

In order to exploit the polypeptide strategy to express multiple genes driven by the same controller element in a CTV based vector, a fusion polypeptide was created consisting of GFP/Protease (Pro)/GUS. Two different protease motifs were used in the different constructs, HC-Pro and NIa, with their proteolytic motifs and recognition sequences separating GFP ORF from the GUS ORF (FIGS. 14A & 3-16) (Carrington and Dougherty, 1988; Carrington et al., 1989). Theoretically, in case the NIa was the protease motif in the fusion, six extra amino acids are coupled with the N-terminal protein (GFP) at its C-terminus whereas only one extra amino acid is added to the N-terminus of GUS. Similarly, where HC-Pro was the protease within the fusion poly-peptide, 7 extra amino acids are added to the C-terminus of GFP and two extra amino acids added to the N-terminus of GUS. The fusion genes ranged in size between 3127 and 3480 nts.

Replacement of p13 Gene

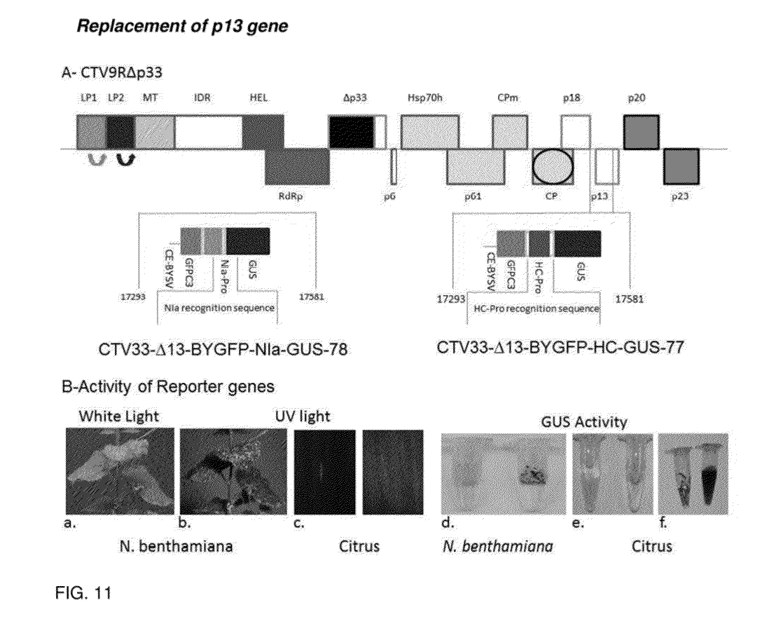

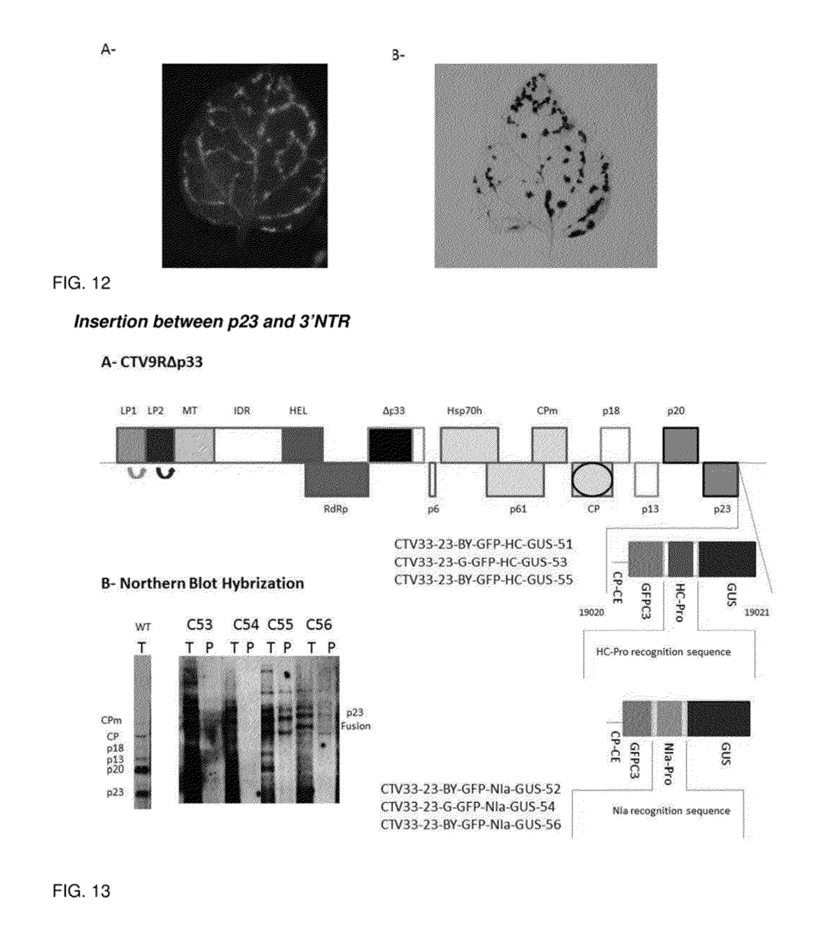

The two fusions of GFP/Pro/GUS described above were engineered into the p13 site of CTV in the agro-inoculation binary vector under the control of the BYSV CP-CE (CTV33-Δ13-BYGFP-HC-GUS-77 with HC-Pro protease motif and CTV33-Δ13-BYGFP-NIa-GUS-78 with NIa protease motif) (FIG. 11A). The constructs were agro-inoculated to N. benthamiana for monitoring the ability to systemically infect the plant and produce GUS and GFP. Both genes were produced based on their assays (FIG. 11B). Western immune-blot analysis indicated the efficient processing of the GFP protein from the polypeptide fusion (FIG. 10). The virus multiplied and spread to high titers in N. benthamiana plants as indicated by symptom development in the upper leaves (FIG. 11B) and ELISA. However, the level of GFP fluorescence was less than that of vectors CTV33-Δ13-BY-GFP-57, CTV33-Δ13-G-GFP-65 and CTV33-Δ13-B-GFP-66 expressing the GFP alone and spread more slowly into the upper non-inoculated leaves than those vectors (Data not presented). In N. benthamiana plants, overlapping fluorescence and enzymatic activity of GUS were demonstrated 7 months after the injection of the construct revealing their stability (FIG. 12).

Insertion Between p23 and 3′NTR

In an attempt to improve the expression level of GFP and GUS, the fusion polypeptide was moved closer to the 3′NTR. The fusion gene with either BYSV, GLRaV-2 or BYV CP-CE with the protease of HC-Pro was inserted between p23 and 3′NTR referred to as CTV33-23-BY-GFP-HC-GUS-51, CTV33-23-G-GFP-HC-GUS-53 and CTV33-23-BY-GFP-HC-GUS-55 whereas with the NIa protease constructs were named, CTV33-23-BY-GFP-NIa-GUS-52, CTV33-23-G-GFP-NIa-GUS-54 and CTV33-23-BY-GFP-NIa-GUS-56, respectively (FIG. 13). After N. benthamiana plants were agro-inoculated, all the constructs multiplied and spread into the upper non-inoculated leaves as indicated by GFP fluorescence (FIG. 14A) and GUS activity (FIG. 14A). Similar to constructs CTV33-Δ13-BYGFP-HC-GUS-77 and CTV33-Δ13-BYGFP-NIa-GUS-78, fluorescence overlapping with GUS enzymatic activity was demonstrated 7 months after injection indicating the stability of the fusion. However, C. macrophylla plants infected with construct CTV33-23-BY-GFP-HC-GUS-51 revealed only faint fluorescence and almost no GUS activity (FIG. 14B) and high ELISA values.

### Example 5

### Use of Multiple Promoters to Express Foreign Genes Simultaneously

Bimolecular Fluorescence Complementation (BiFC) in CTV.

For examination of the insertion of two CP-CE controlling different ORFs, the BiFC system, which produces visible fluorescence only when the two proteins accumulate in the same cell, was used. This system was developed using the bJun fused to N-terminus of EYFP (A.A. 1-154) (referred to as bJunN) and bFos ORF fused to C-terminus of EYFP (A.A. 155-238) (referred to as bFosC) (Hu et al., 2002).

Both proteins are transported to the nucleus where they directly interact enabling the EYFP protein to regain its wild type folding pattern and results in emission of fluorescence upon activation by a blue light source (Excitation wave length is 525 nm and emission wavelength is 575 nm) (Hu et al., 2002). One or both components of BiFC were introduced into the CTV mini-replicon 3′ of the p23 ORF (between nts #19020 and 19021 Genbank Accession #AY170468) referred to as CTVp333R-23-BYbJunN, CTVp333R-23-GbFosC and CTVp333R-23-BYbJunN-GbFosC (FIG. 15A). Northern blot hybridization analysis demonstrates the successful transfection of all three constructs into N. benthamiana protoplast (FIG. 15B). The two transcription factors interacted in the plant cell as demonstrated by nuclear fluorescence observed only in protoplasts infected with CTVp333R-23-BYbJunN-GBFosC (FIG. 15C). It is worth noting that the size of the two inserted genes is approximately identical to that of the GUS ORF.

As a control for the BiFC experiments, the genes were introduced individually into Δp33CTV9R behind p23 creating vectors CTV33-23-BYbJunN-97 and CTV33-23-GbFosC-98 so that only one component would be produced (FIG. 16B). Neither construct exhibited fluorescence in the nucleus.

Expression of Multiple Foreign Genes Simultaneously at the Same Location

P13 Replacement.

Both genes were introduced into a Δp33CTV9R (Satyanarayana et al., 1999, 2000, 2003; Tatineni et al., 2008) as a replacement of the p13 gene (replacement of the nucleotides deleted between 17292 and 17581), resulting in CTV33-Δ13-BYbJunN-GbFosC-76 (FIG. 16A). Transfection of protoplasts with the RNA transcripts of CTV33-Δ13-BYbJunN-GbFosC-76 resulted in the nuclear fluorescence of infected protoplasts (Data not presented). Similarly, infiltrated leaves of N. benthamiana plants with full length CTV33-Δ13-BYbJunN-GbFosC-76 emitted nuclear fluorescence (FIG. 16B). On the contrary, infiltrated leaves with constructs CTV33-23-BYbJunN-97 and CTV33-23-GbFosC-98 did not show any nuclear fluorescence (Data not presented). Monitoring stem phloem and leaf veins of N. benthamiana plants infiltrated with CTV33-Δ13-BYbJunN-GbFosC-76 seven weeks after infiltration revealed fluorescence of the vascular tissue indicating the ability of this construct to systemically infect upper leaves of N. benthamiana (FIG. 16B).

Insertion Between p23 and 3′NTR.

The next step was to examine expression of the two genes when positioned closer to the 3′ terminus. The two gene components of the BiFC system were introduced into CTVΔp33behind p23 (between nts #19020 and 19021), CTV33-23-BYbJunN-GbFosC-59 (FIG. 3-17A). Upon RNA transfection of construct CTV33-23-BYbJunN-GbFosC-59, nuclear flourescence of infected protoplast was observed under the fluorescent microscope. However, it was difficult to pass the new construct from one protoplast batch to another, similar to GUS and the GFP/Pro/GUS fusion genes inserted at the same location. Upon agro-infiltration of N. benthamiana plants with CTV33-23-BYbJun-GbFosC-59 in full length CTV, fluorescence was observed in infiltrated areas. Systemic symptoms similar to that expected for infection of N. benthamiana by CTV was extremely delayed. However, monitoring upper non-inoculated leaves and phloem tissue of the stem at seven weeks after agro-infiltration of leaves revealed fluorescence of nuclei of the vascular tissue, demonstrating systemic infection by the vector (FIG. 17C). These results confirmed by ELISA, indicate that the position between p23 and 3′NTR can accommodate two extra genes without affecting the ability of CTV to systemically invade the plants. Similar to both genes replacing p13 in construct CTV33-Δ13-BYbJunN-GbFosC-76 there was a delay in the time frame of colonizing the upper vascular tissues by construct CTV33-23-BYbJunN-GbFosC-59. Nuclear fluorescence of systemic stem phloem tissue indicates that CTV33-Δ13-BYbJunN-GbFosC-76 infected more cells than construct CTV33-23-BYbJunN-GbFosC-59 (FIG. 16B & FIG. 17C). This difference in the number of cells infected indicates the better ability of CTV33-Δ13-BYbJunN-GbFosC-76 to move in N. benthamiana as compared to CTV33-23-BYbJunN-GbFosC-59.

### Example 6

### Expression of Multiple Foreign Genes Simultaneously from Different Locations

To express multiple foreign genes from two different positions, p13 gene was placed and a second gene was inserted behind p23. CTV33-Δ13-BYbJunN-23-GbFosC-67 (FIG. 17A) was created via replacement of the p13 gene with the BYSV CP-CE driving the bJunN ORF and the GLRaV-2 CP-CE controlling the bFosC ORF inserted between the p23 ORF and the 3′NTR. CTV33-Δ13-BYbJunN-23-GbFosC-67 was transfected into protoplasts and Northern blot analysis revealed the replication of the virus (FIG. 17B). However, accumulation of the p23 mRNA was greatly reduced. CTV33-Δ13-BYbJunN-23-GbFosC-67 was agro-inoculated into N. benthamiana. The infiltration into the leaves indicated nuclear fluorescence of infected cells (FIG. 17C) which were much fewer in number compared to constructs CTV33-Δ13-BYbJunN-GbFosC-76 and CTV33-23-BYbJunN-GbFosC-59. Isolation of virions from leaves and transfection of protoplast was carried out resulting in nuclear fluorescence of infected protoplast indicating the successful formation of biologically active virions. However, systemic infection was not achieved in N. benthamiana as indicated by the lack of nuclear fluorescence in the stem and upper non-inoculated leaves of N. benthamiana and confirmed by ELISA.

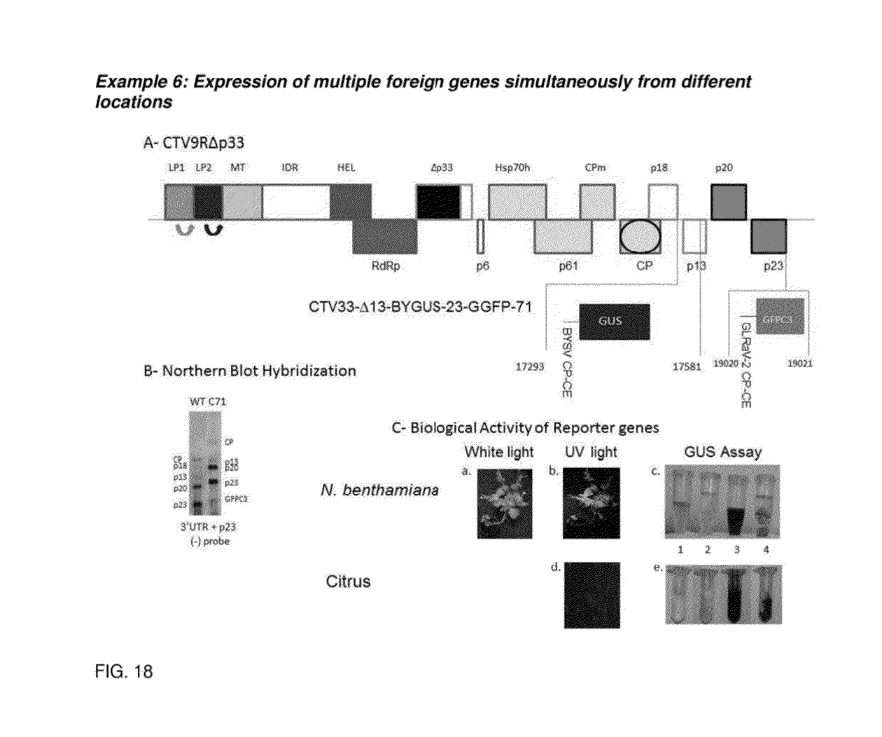

In order to further study simultaneous multiple gene expression from the different locations as above, CTV33-Δ13-BYGUS-23-GGFP-71 was engineered such that the GUS ORF under the control of the BYSV CP-CE replaced the p13 gene(nts 17292-17582) and the GFP ORF under the control of the GLRaV-2 CP-CE was inserted between the p23 and 3′NTR (nts 19020 and 19021) (FIG. 18A). RNA transcripts of CTV33-Δp13-BYGUS-23-GGFP-71 were transfected into N. benthamiana protoplasts and northern blot analysis indicated efficient replication of the construct in protoplasts (FIG. 18B). Leaf infiltration of N. benthamiana plants with construct CTV33-Δp13-BYGUS-23-GGFP-71 resulted in replication of the virus as indicated by visible fluorescence under a UV light and by GUS activity (Data not presented). The agro-inoculated plants began to exhibit GUS activity and fluorescence in the upper non-inoculated leaves 6 weeks after infiltration (FIG. 3-18C). The systemic infection of upper leaves was slightly slower than constructs with only GFP alone. Also, the phenotype of vein clearing followed by necrosis associated with CTV infection of N. benthamiana vascular tissue occurred later than that of single gene vectors. The level of fluorescence when observed UV light appeared to be slightly less than that of the single gene constructs. However, the GFP fluorescence was more in plants infected with construct CTV33-Δp13BYGUS-23GGFP-71, which was controlled by its own CE, compared to that of the fusion in constructs (CTV33-23-BY-GFP-HC-GUS-51, CTV33-23-BY-GFP-NIa-GUS-52, CTV33-23-G-GFP-HC-GUS-53, CTV33-23-G-GFP-NIa-GUS-54, CTV33-Δ13-BYGFP-HC-GUS-77 and CTV33-Δ13-BYGFP-NIa-GUS-78). The activity of both genes continued until the death of the N. benthamiana plants. Similarly, in citrus the expression of both genes were better than the same genes in constructs CTV33-Δ13-BYGFP-NIa-GUS-78 and CTV33-23-BY-GFP-HC-GUS-51.

### Example 7

### Level of Foreign Gene Expression of the Different Constructs in Citrus

It is difficult to directly compare foreign gene expression from the different vectors in citrus due to the differences in the times of infection, the ages of the tissue and the effects of the inserted foreign gene cassette on the replication of the virus. Yet, protein presence in citrus is the best measure of expression level. Thus, western blot analysis was used to compare the relative level of expression of the different GFP and GUS constructs in citrus to that of CP protein, a house keeping gene to determine the replication levels. Western blots using the GFP antibodies and the CP antibody revealed a trend which confirms the relative higher expression levels near the 3′end of the genome and a lower expression level when the inserted gene is moved further away from the 3′end with the exception for the insertion between p13 and p20 (FIG. 19A). In contrary, the GUS expression in citrus revealed a higher relative expression level as replacement of p13 rather than insertion behind p23 (FIG. 19B).

### Example 8

### Multiple Gene Vectors

Plasmid Construction:

Three and four gene vectors were developed by introducing different combination of gene cassettes into the CTV genome at different locations. Three of the vectors were developed in CTV9RΔp33in the pCAMBIA 1380 background (CTV33-BGFP-BYGUS-GTMVCP-79, CTV33-BGFP-GbFosC-BYbJunN-81 and CTV33-Δ13-BGFP-BYbJunN-GbFosC-82). The other three three gene vectors (CTV-BASL-BYPTA-CP7-119, CTV-BASL-BYP10-CP7-131, CTV-BASL-BYPTA-CP10-120 and CTV-BRFP-BYGFP-CTMVCP-117) and one four gene vector (CTVΔ13-BRFP-GbFosC-BYbJunN-CTMVCP-118) were developed by modifying CTV9R in the background of pCAMBIΔ1380 altered by replacing the hygromycin ORF with the p22 ORF of Tomato chlorosis virus. For the ease of cloning the PstI restriction site in p33 ORF in full length CTV9R was eliminated by introducing a silent mutation using overlap extension PCR using primers 1749 and 1750 in combination with primer C-1436 and C-253 followed by digestion of both the overlap PCR product and CTV9R with XmaI and PmeI. Most of the gene cassettes were introduced into their locations by overlap extension PCR using the primers listed in table 1. The only exception was the insertion of green fluorescent protein cycle 3 in between the CPm and CP gene. Introducing the GFPC3 gene cassette into that location was done by restriction digestion of 9-47RGFP plasmid and point mutated CTV9R in pCAMBIΔ1380 with PmeI and PstI.

Expression of Three and Four Foreign Genes Simultaneously

After successfully expressing two genes in N. benthamiana and citrus with one and two different controller elements we are building vectors to express three and four foreign genes from three and four different controller elements, respectively. The reporter genes used in different combinations were the green fluorescent protein (cycle 3 GFP, GFPC3), red fluorescent protein (tag red fluorescent protein, RFP), Bimolecular fluorescence complementation using the bFos and bJun mammalian transcription factors (Hu et al., 2002), β-glucuronidase (GUS) gene from Escherichia coli and the Tobacco mosaic virus (TMV) coat protein gene (CP). Similarly, three gene vectors were built in different combinations to express two antimicrobial peptides (AMPs) from Tachypleus tridentatus and Sus scorfa, Allium sativum lectin (ASL) and Pinellia ternata agglutinin (PTA). The three gene vectors were either expressed from two or three locations within the CTV genome

Expression of Three Foreign Genes from Three Different Locations Simultaneously:

Six vectors were built to express three foreign genes from three different locations. The vectors were built to express the genes either from CTV9RΔp33or full length CTV9R.

Vectors Built to Express Three Genes from Three Different Locations in CTV9RΔp33

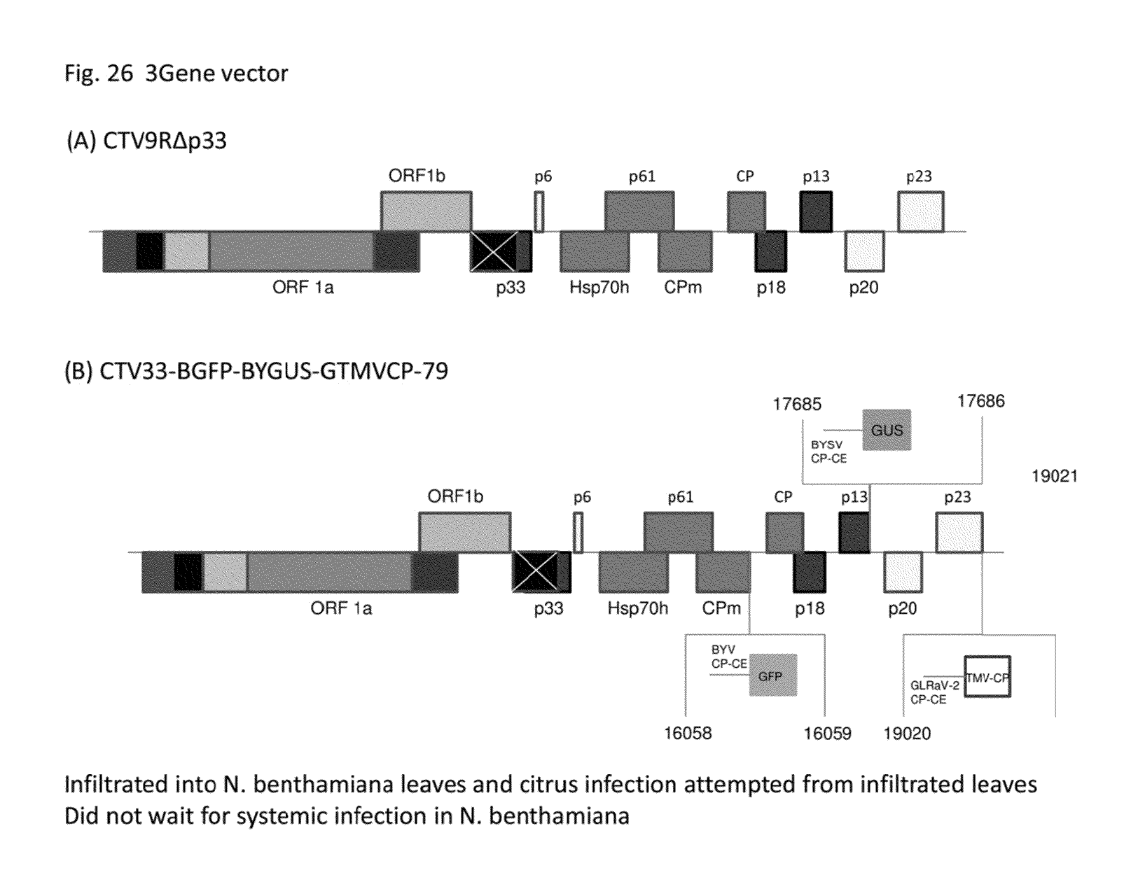

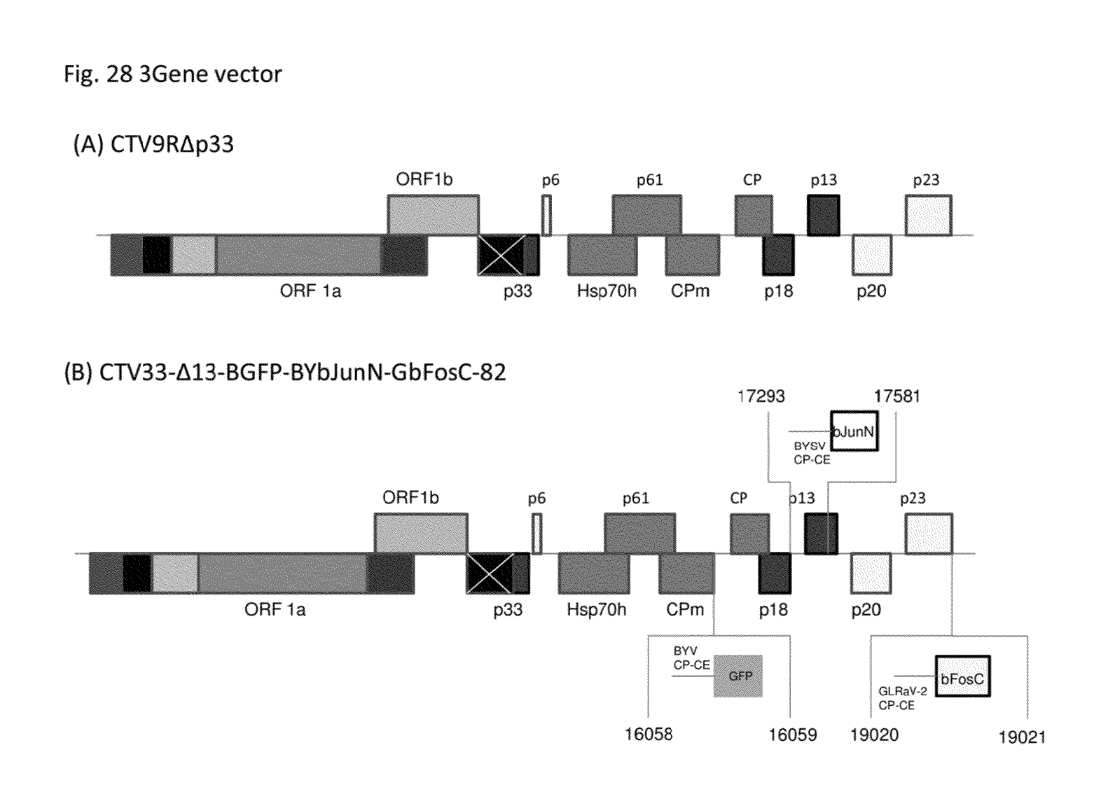

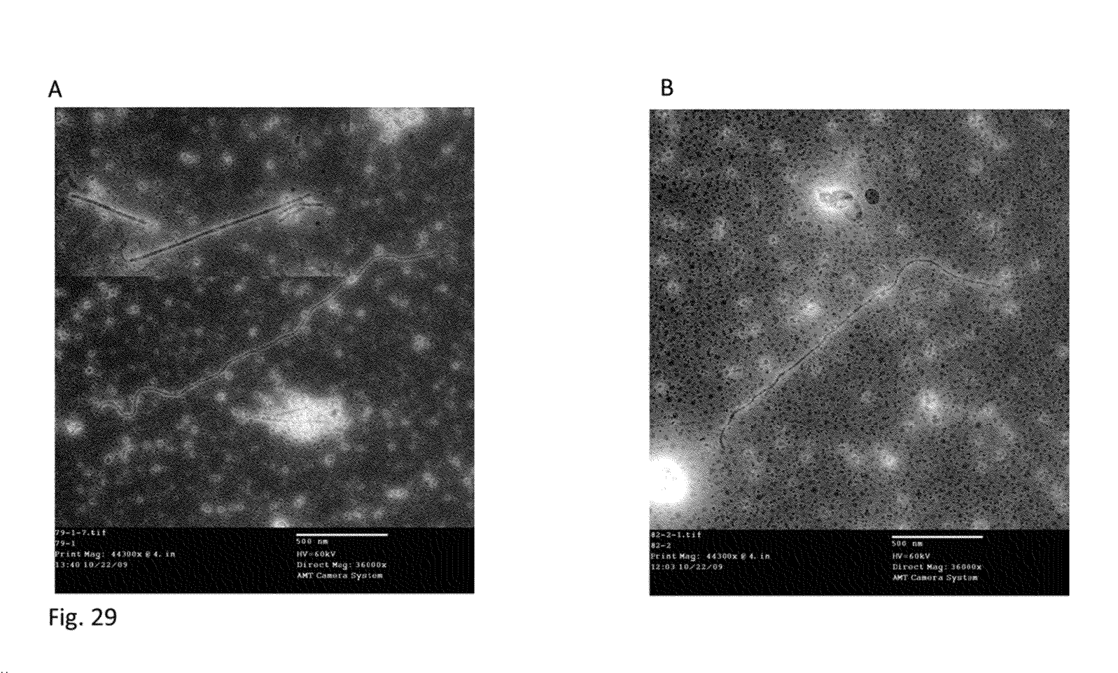

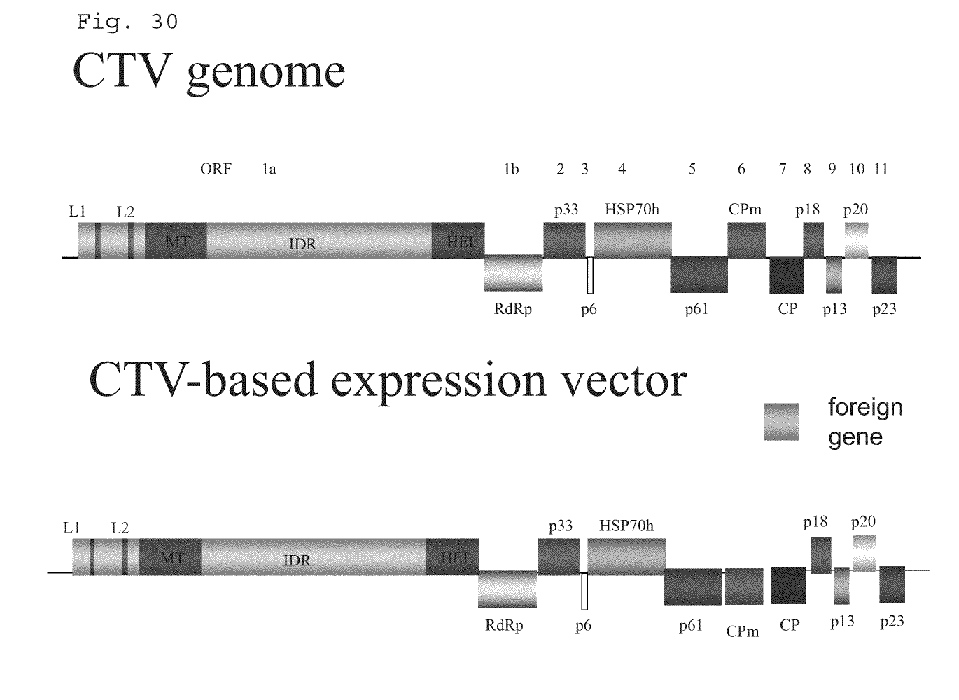

Two vectors were built by inserting the three extra gene cassettes into CTV9RΔp33 creating expression vectors CTV33-BGFP-BYGUS-GTMVCP-79 (FIG. 26) and CTV33-Δ13-BGFP-BYbJunN-GbFosC-82 (FIG. 28). CTV33-BGFP-BYGUS-GTMVCP-79 expresses the three ORFs of GFP (insertion between CPm and CP), GUS (insertion between p13 and p20) and the coat protein of TMV (insertion between p23 and 3′UTR) under the CP-CE of BYV, BYSV and GLRaV-2, respectively. CTV33-Δ13-BGFP-BYbJunN-GbFosC-82 expresses the three ORFs of GFP (insertion between CPm and CP), bJunN ORF (replacement of p13) and bFosC (insertion between p23 and 3′UTR) under the CP-CE of BYV, BYSV and GLRaV-2, respectively. The two vectors were infiltrated into N. benthamiana leaves in combination with silencing suppressors and inoculated into citrus using the procedure of Gowda et al., 2005. As leaves were cut and grinded to isolate virions over 70% sucrose cushion gradient just 5 days after infiltration into the N. benthamiana leaves it was not likely that these plants will get systemically infected, thus they were discarded. The fluorescence of infiltrated leaves under hand held UV indicated the expression of the GFP protein in both CTV33-BGFP-BYGUS-GTMVCP-79 and CTV33-Δ13-BGFP-BYbJunN-GbFosC-82 indicating the ability of the created vector to replicate in the N. benthamiana leaves. Electron microscope grids prepared from leaf dips of infiltrated N. benthamiana leaves for construct CTV33-BGFP-BYGUS-GTMVCP-79 and CTV33-Δ13-BGFP-BYbJunN-GbFosC-82 indicated the formation of virions a prerequisite for the successful mechanical inoculation of citrus seedlings with CTV. Furthermore, in the case of CTV33-BGFP-BYGUS-GTMVCP-79 and not CTV33-Δ13-BGFP-BYbJunN-GbFosC-82 there was the formation of rod-shaped structures referred to as TMV pseudo-virions a characteristic of the expression of the TMV coat protein.

Vectors Built to Express Three Genes from Three Different Locations in CTV9R

Four vectors were built to express three foreign genes from the same three different locations within the CTV genome. The three locations selected were insertion between CPm and CP, p13 and p20 and p23 and 3′UTR. For the ease of cloning into the full length CTV infectious clone a the PstI site within the p33 ORF was eliminated by introducing a silent point mutation by overlap extension PCR. Three of the four vectors were created by using different combinations of the two AMPs, ASL and PTA resulting in expression vectors CTV-BASL-BYPTA-CP7-119, CTV-BASL-BYP10-CP7-131 and CTV-BASL-BYPTA-CP10-120. The fourth vector named CTV-BRFP-BYGFP-CTMVCP-117 was created by inserting the ORFs of GFP, RFP and TMV CP under the control of BYV, BYSV and duplicated CP-CE of CTV. All the vectors were infiltrated into N. benthamiana to monitor the development of systemic infection. CTV-BASL-BYPTA-CP7-119 developed efficient systemic infection in 1 N. benthamiana plant. Plants infiltrated with vector CTV-BRFP-BYGFP-CTMVCP-117 revealed fluorescence in systemic leaves under hand held UV. Upon development of pronounced systemic infection, virions from CTV-BRFP-BYGFP-CTMVCP-117 will be concentrated over a sucrose step gradient and a sucrose cushion in order to inoculate citrus plants similar to the procedure recently followed for vector CTV-BASL-BYPTA-CP7-119

Expression of Three Foreign Genes from Two Different Locations Simultaneously:

Two vectors were created for the simultaneous expression of three genes from two different locations within the CTV genome. One vector was built in CTV9RΔp33creating expression vector CTV33-BGFP-GbFosC-BYbJunN-81 whereas the other vector was built in full length CTV9R named CTVΔ13-GbFosC-BYbJunN-CTMVCP-129.

Vector Built to Express Three Genes from Two Different Locations in CTV9RΔp33:

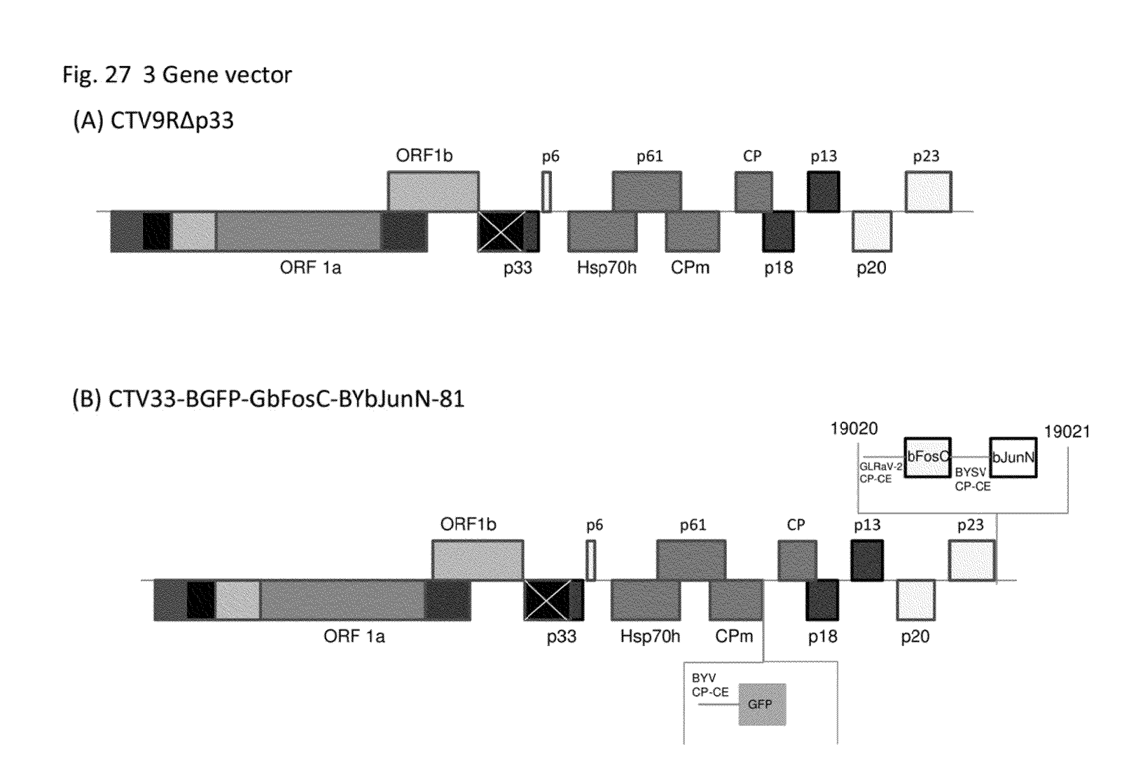

CTV33-BGFP-GbFosC-BYbJunN-81 (FIG. 27) was engineered through modifying CTV9RΔp33by inserting a single gene cassette between CPm and CP (GFP ORF under the control of BYV CP-CE) and a double gene cassette (bFosC ORF followed by bJunN ORF under the control of GLRaV-2 and BYSV CP-CE, respectively) as an insertion between p23 and 3′UTR. A 1:1 mixture of 4 different silencing suppressors and CTV33-BGFP-GbFosC-BYbJunN-81 were infiltrated into N. benthamiana leaves. Electron microscopy from grids of leaf dips revealed the formation of virions similar to constructs CTV33-BGFP-BYGUS-GTMVCP-79 and CTV33-Δ13-BGFP-BYbJunN-GbFosC-82. In addition, the infiltrated leaves revealed strong fluorescence under hand held UV light. Infiltrated leaves were used to concentrate virions on a 70% sucrose cushion in an attempt to infect citrus seedlings.

Vector Built to Express Three Genes from Two Different Locations in CTV9R:

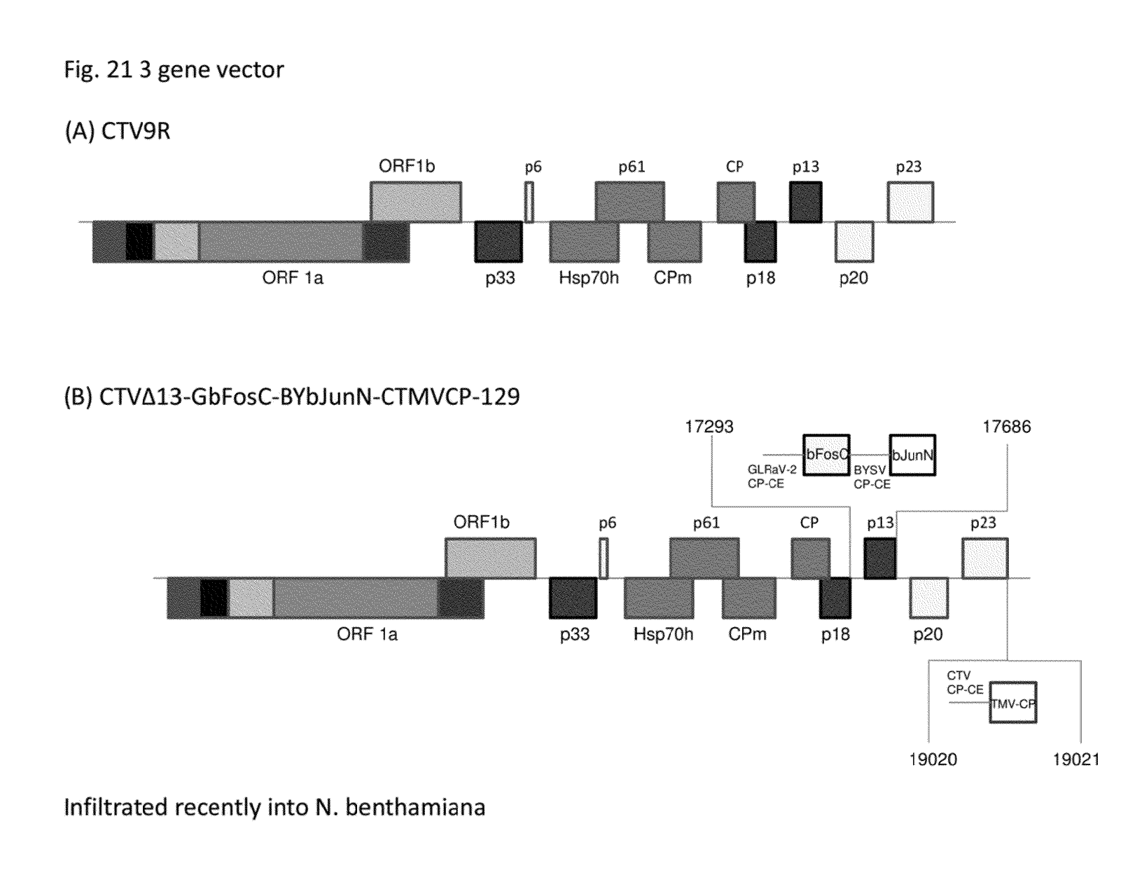

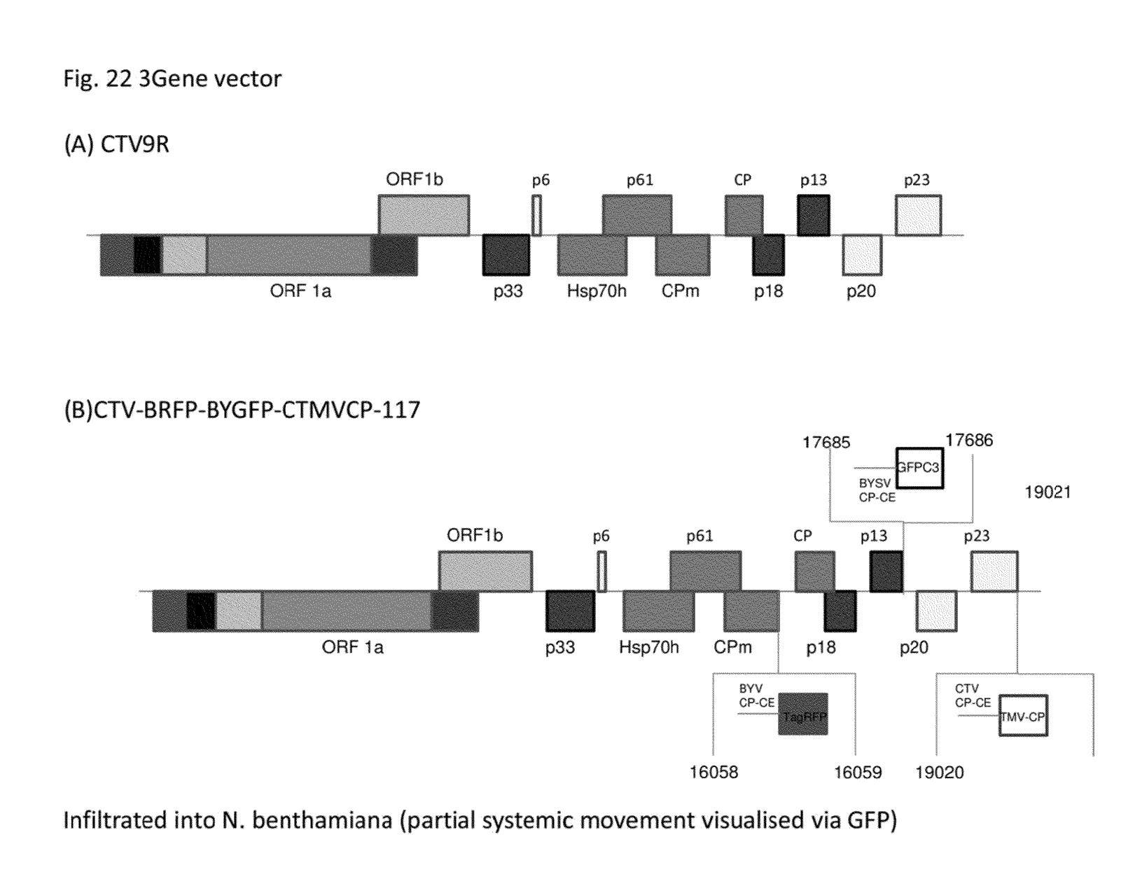

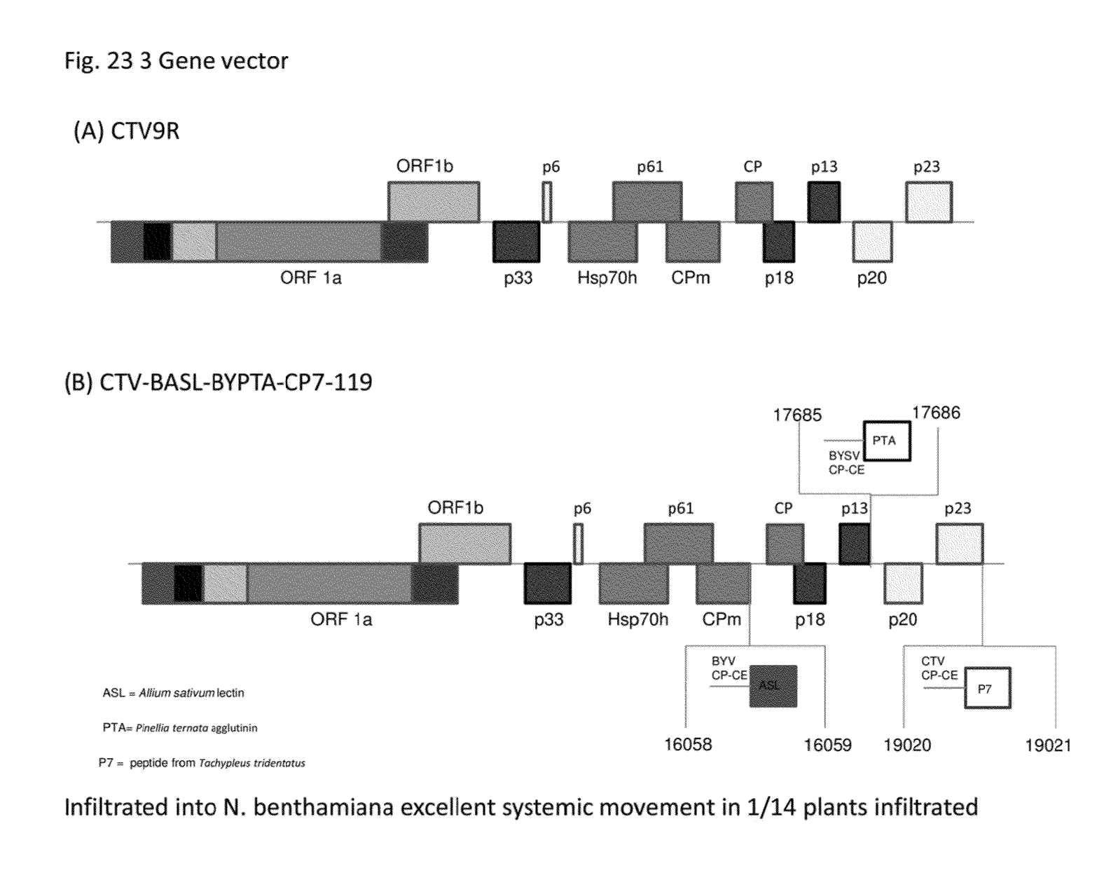

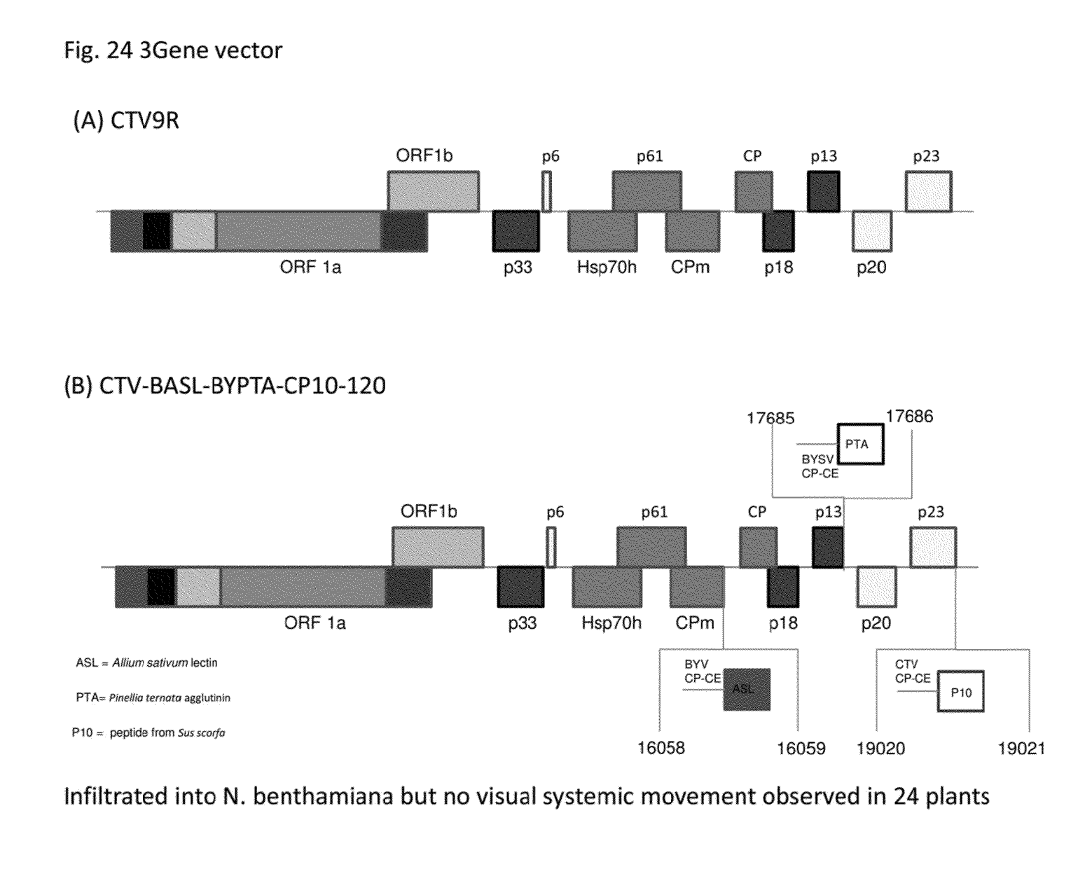

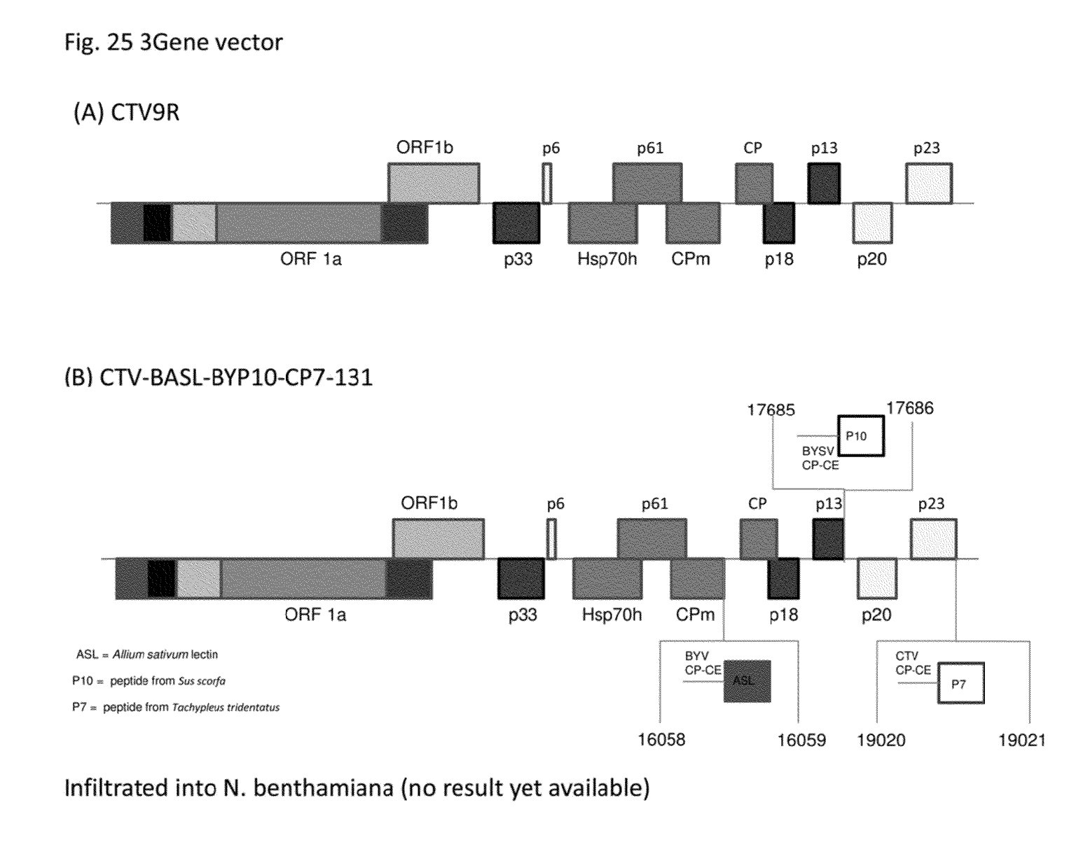

CTV9R was modified by inserting a double gene cassette (bFosC ORF followed by bJunN ORF under the control of GLRaV-2 and BYSV CP-CE, respectively) as replacement of p13 and a gene cassette (TMV CP ORF under the control of the duplicated CP-CE) as an insertion between p23 and 3′UTR creating expression vector CTVΔ13-GbFosC-BYbJunN-CTMVCP-129 (FIG. 21). This vector is recently infiltrated into N. benthamiana leaves. After systemic infection of N. benthamiana the virions will be concentrated to enable the inoculation of citrus plants.

Expression of Four Foreign Genes from Three Different Locations Simultaneously:

In order to build the four gene vector we used four gene cassettes located at three different locations within the CTV genome. The RFP ORF was introduced between CPm and CP under the control of the BYV CP-CE, the two BiFC components bFosC and bJunN under the control of GLRaV-2 and BYSV respectively were introduced as a replacement of the p13 gene and the TMV ORF under the control of the duplicated CP-CE of CTV was introduced behind p23. The four gene vector named CTVΔ13-BRFP-GbFosC-BYbJunN-CTMVCP-118 was infiltrated into the N. benthamiana leaves for the development of systemic infection. Upon systemic infection virion concentration will be carried out over a sucrose step gradient and cushion for the infection of the citrus trees.

Spinach Defensin Genes

I. Compositions

A. Antimicrobial Peptides

The present disclosure relates, one or more antimicrobial-peptides belonging to the family of plant defensins. These polypeptides were originally isolated from spinach leaves (Spinacia oleracea). In some embodiments, a defensin may be small (about 5 kDa), may be basic and/or may be cysteine-rich. In some embodiments, a defensin may comprise a peptide having an amino acid sequence sharing at least about 95% identity, at least about 96% identity, at least about 97% identity, at least about 98% identity, at least about 99% identity, and/or about 100% identity with SEQ ID NO: 1 and/or SEQ ID NO: 2. In some embodiments, an antimicrobial peptide may further comprise one or more amino acids that are independently and/or collectively either neutral (e.g., do not adversely impact antibacterial functionality) and/or augment antibacterial functionality (e.g., by directing the peptide to a desired location (e.g., cellular and/or extracellular). For example, a defensin may comprise a signal peptide derived from the tobacco pathogenesis-related (PR)-1b protein that allows the transport of the peptides into the apoplast of plant cells (e.g., via the secretory pathway) and/or accumulation in the intercellular spaces of leaves, stems, flowers, fruits, seeds, and/or roots. A defensin may comprise, according to some embodiments, a peptide having an amino acid sequence sharing at least about 95% identity, at least about 96% identity, at least about 97% identity, at least about 98% identity, at least about 99% identity, and/or about 100% identity with SEQ ID NO: 7 and/or SEQ ID NO: 8.

B. Nucleic Acids

The present disclosure relates, in some embodiments, to nucleic acids (e.g., cassettes, vectors) comprising one or more sequences encoding one or more antimicrobial peptides. For example, a nucleic acid may comprise a cassette comprising a synthetic nucleic acid sequence of SoD2 and/or SoD7 genes. Synthetic SoD2 and/or SoD7 codons may specify the same amino acid sequences as native spinach, having their codons optimized for citrus codon usage. A nucleic acid comprising a SoD2 and/or SoD7 coding sequence may comprise a sequence encoding a signal peptide (e.g., PR-1b). In some embodiments, expression of a nucleic acid comprising a sequence encoding an antimicrobial peptide may be optimized by positioning an initiation codon in a favorable (e.g., optimal) 5′ context. According to some embodiments, a nucleic acid may comprise an expression control sequence (e.g., operably linked to a coding sequence). For example, a nucleic acid may comprise a coding gene sequence under the control of a dual enhanced CaMV 35S promoter with a 5′ UTR from TEV plant potyvirus (e.g., to provide a translation-enhancing activity to the defensin genes).

According to some embodiments, a nucleic acid may comprise a nucleotide sequence having at least about 75% identity to SEQ ID NOS: 3, 4, 5, 6, 9, 10, 11, 12, 29, 30, and/or 31, at least about 80% identity to SEQ ID NOS: 3, 4, 5, 6, 9, 10, 11, 12, 29, 30, and/or 29, at least about 85% identity to SEQ ID NOS: 3, 4, 5, 6, 9, 10, 11, 12, 29, 30, and/or 31, at least about 90% identity to SEQ ID NOS: 3, 4, 5, 6, 9, 10, 11, 12, 29, 30, and/or 31, at least about 95% identity to SEQ ID NOS: 3, 4, 5, 6, 9, 10, 11, 12, 29, 30, and/or 31, at least about 97% identity to SEQ ID NOS: 3, 4, 5, 6, 9, 10, 11, 12, 29, 30, and/or 31, at least about 98% identity to SEQ ID NOS: 3, 4, 5, 6, 9, 10, 11, 12, 29, 30, and/or 31, at least about 99% identity to SEQ ID NOS: 3, 4, 5, 6, 9, 10, 11, 12, 29, 30, and/or 31, and/or about 100% identity to SEQ ID NOS: 3, 4, 5, 6, 9, 10, 11, 12, 29, 30, and/or 31. A nucleotide sequence may encode, in some embodiments, an amino acid sequence having at least about 98% identity to SEQ ID NOS: 1, 2, 7, 8, and/or 28, at least about 99% identity to SEQ ID NOS: 1, 2, 7, 8, and/or 28, and/or about 100% identity to SEQ ID NOS: 1, 2, 7, 8, and/or 28. According to some embodiments, a nucleic acid may have a first measure of sequence identity to a reference nucleic acid sequence and may encode an amino acid sequence having a second measure of sequence identity to a reference amino acid sequence. For example, a nucleic acid may have about 85% identity to SEQ ID NOS: 3, 4, 5, 6, 9, 10, 11, 12, 29, 30, and/or 31 and encode an amino acid sequence having about 100% identity with SEQ ID NOS: 1, 2, 7, 8, and/or 28, according to some embodiments.

A nucleic acid sequence, according to some embodiments, may hybridize to a nucleic acid having the nucleotide sequence of SEQ ID NOS: 3, 4, 5, 6, 9, 10, 11, 12, 29, 30, and/or 31 under stringent conditions. Stringent conditions may include, for example, (a) 4×SSC at 65° C. followed by 0.1×SSC at 65° for 60 minutes and/or (b) 50% formamide, 4×SSC at 65° C. A nucleic acid may comprise a deletion fragment (e.g., a deletion of from about 1 to about 12 bases) of a nucleic acid having a sequence of SEQ ID NOS: 3, 4, 5, 6, 9, 10, 11, 12, 29, 30, and/or 31 that retains antimicrobial activity against at least one microorganism capable of infecting a citrus plant. One of ordinary skill in the art having the benefit of the present disclosure may prepare one or more deletion fragments of a nucleic acid having a sequence of SEQ ID NOS: 3, 4, 5, 6, 9, 10, 11, 12, 29, 30, and/or 31 and screen the resulting fragments for antimicrobial activity against at least one microorganism capable of infecting a citrus plant.

A nucleic acid sequence having a sequence like SEQ ID NOS: 3, 4, 5, 6, 30, and/or 31 may be identified by database searches using the sequence or elements thereof as the query sequence using the Gapped BLAST algorithm (Altschul et al., 1997 Nucl. Acids Res. 25:3389-3402) with the BLOSUM62 Matrix, a gap cost of 11 and persistence cost of 1 per residue and an E value of 10. Sequence identity may be assessed by any available method according to some embodiments. For example, two sequences may be compared with either ALIGN (Global alignment) or LALIGN (Local homology alignment) in the FASTA suite of applications (Pearson and Lipman, 1988 Proc. Nat. Acad. Sci. 85:2444-2448; Pearson, 1990 Methods in Enzymology 183:63-98) with the BLOSUM50 matrix and gap penalties of −16, −4. Sequence similarity may be assessed according to ClustalW (Larkin et al., 2007, Bioinformatics 23(21): 2947-2948), BLAST, FASTA or similar algorithm.

C. Expression Cassettes and Vectors

In some embodiments, to expression vectors and/or expression cassettes for expressing a nucleic acid sequence (e.g., a coding sequence) in a cell and comprising an expression control sequence and the nucleic acid sequence operably linked to the expression control sequence. Thus, for example, an expression cassette may comprise a heterologous coding sequence, the expression of which may be desired in a plant. Preferred expression vectors and cassettes are described above regarding CTV vectors.

1. Expression Vectors

The disclosure relates, in some embodiments, to an expression vector which may comprise, for example, a nucleic acid having an expression control sequence and a coding sequence operably linked to the expression control sequence. In some embodiments, an expression control sequence may comprise one or more promoters, one or more operators, one or more enhancers, one or more ribosome binding sites, and/or combinations thereof. An expression control sequence may comprise, for example, a nucleic acid having promoter activity. An expression control sequence, according to some embodiments, may be constitutively active or conditionally active in (a) an organ selected from root, leaf, stem, flower, seed, and/or fruit, and/or (b) active in a tissue selected from epidermis, periderm, parenchyma, collenchyma, sclerenchyma, xylem, phloem, and/or secretory structures. An expression control sequence, according to some embodiments, may be operable to drive expression of a nucleic acid sequence (e.g., a coding sequence) in a cell. Metrics for expression may include, for example, rate of appearance and/or accumulation of a gene product (e.g., RNA and/or protein) and/or total accumulation of a gene product as of one or more time points (e.g., elapsed time after a starting point and/or a stage of development). Comparative assays for gene products may be qualitative, semi-quantitative, and/or quantitative in some embodiments. Comparative assays may indirectly and/or directly assess the presence and/or amount of gene product. In some embodiments, an expression control sequence may be sensitive to one or more stimuli (e.g., one or more small molecules, one or more plant defense-inducing agents, mechanical damage, temperature, pressure). For example, activity of an expression control sequence may be enhanced or suppressed upon infection with a microorganism (e.g., a bacteria or a virus).

An expression vector may be contacted with a cell (e.g., a plant cell) under conditions that permit expression (e.g., transcription) of the coding sequence. Examples of expression vectors may include the Agrobacterium transformation constructs shown in FIG. 1 and FIG. 2. An expression control sequence may be contacted with a plant cell (e.g., an embryonic cell, a stem cell, a callous cell) under conditions that permit expression of the coding sequence in the cell and/or cells derived from the plant cell according to some embodiments. An expression vector may be contacted with a cell (e.g., a plant cell), in some embodiments, under conditions that permit inheritance of at least a portion of the expression vector in the cell's progeny. According to some embodiments, an expression vector may include one or more selectable markers. For example, an expression vector may include a marker for selection when the vector is in a bacterial host, a yeast host, and/or a plant host.

2. Expression Cassettes

According to some embodiments, the disclosure relates to an expression cassette which may comprise, for example, a nucleic acid having an expression control sequence and a coding sequence operably linked to the expression control sequence. An expression cassette may be comprised in an expression vector. A coding sequence, in some embodiments, may comprise any coding sequence expressible in at least one plant cell. For example, a coding sequence may comprise a plant sequence, a yeast sequence, a bacterial sequence, a viral sequence (e.g., plant virus), an artificial sequence, an antisense sequence thereof, a fragment thereof, a variant thereof, and/or combinations thereof. A coding sequence may comprise, in some embodiments, a sequence encoding one or more gene products with insecticidal, antibacterial, antifungal, antimicrobial, and/or antiviral activity. A coding sequence may comprise, in some embodiments, a start codon, an intron, and/or a translation termination sequence. According to some embodiments, a coding sequence may comprise one or more natural or artificial coding sequences (e.g., encoding a single protein or a chimera). According to some embodiments, an expression cassette may optionally comprise a termination sequence. A coding sequence, in some embodiments, may comprise a sequence at least partially codon optimized for expression in an organism of interest (e.g., a citrus plant).

An expression control sequence may be used, in some embodiments, to construct an expression cassette comprising, in the 5′ to 3′ direction, (a) the expression control sequence, (b) a heterologous gene or a coding sequence, or sequence complementary to a native plant gene under control of the expression control sequence, and/or (c) a 3′ termination sequence (e.g., a termination sequence comprising a polyadenylation site). Examples of expression cassettes may include, in some embodiments, the cassettes shown in SEQ ID NOS: 13-16. An expression cassette may be incorporated into a variety of autonomously replicating vectors in order to construct an expression vector. An expression cassette may be constructed, for example, by ligating an expression control sequence to a sequence to be expressed (e.g., a coding sequence).

Some techniques for construction of expression cassettes are well known to those of ordinary skill in the art. For example, a variety of strategies are available for ligating fragments of DNA, the choice of which depends on the nature of the termini of the DNA fragments. An artisan of ordinary skill having the benefit of the present disclosure, a coding sequence (e.g., having antimicrobial activity) and/or portions thereof may be provided by other means, for example chemical or enzymatic synthesis. A nucleic acid may comprise, in a 5′ to 3′ direction, an expression control sequence, a linker (optional), and a coding sequence according to some embodiments. A nucleic acid may comprise, in some embodiments, one or more restriction sites and/or junction sites between an expression control sequence, a linker, and/or a coding sequence.

II. Microorganisms

The present disclosure relates, in some embodiments, to a microorganism comprising an antimicrobial peptide (e.g., a heterologous antimicrobial peptide) and/or a nucleic acid (e.g., a heterologous and/or expressible nucleic acid) comprising a nucleic acid sequence encoding an antimicrobial peptide. For example, a microorganism may comprise a bacteria, a yeast, and/or a virus. Examples of microorganisms may include, without limitation, Agrobacterium tumefaciens, Escherichia coli, a lepidopteran cell line, a Rice tungro bacilliform virus, a Commelina yellow mosaic virus, a Banana streak virus, a Taro bacilliform virus, and/or baculovirus. According to some embodiments, an antimicrobial peptide may be tolerated by and/or innocuous to its host microorganism. A microorganism may comprise an expression control sequence and an antimicrobial peptide coding sequence operably linked to the expression control sequence. A nucleic acid (e.g., a heterologous and/or expressible nucleic acid) comprising a nucleic acid sequence encoding an antimicrobial peptide may be present, in some embodiments, on a genomic nucleic acid and/or an extra-genomic nucleic acid.

III. Plants

The present disclosure relates, in some embodiments, to a plant cell (e.g., an embryonic cell, a stem cell, a callous cell), a tissue, and/or a plant comprising an antimicrobial peptide (e.g., a heterologous antimicrobial peptide) and/or a nucleic acid (e.g., a heterologous and/or expressible nucleic acid) comprising a nucleic acid sequence encoding an antimicrobial peptide. A plant and/or plant cell may be a dicot in some embodiments. Examples of a dicot may include, without limitation, coffee, tomato, pepper, tobacco, lima bean, Arabidopsis, rubber, orange, grapefruit, lemon, lime, tangerine, mandarin, pummelo, potato, squash, peas, and/or sugar beet. A plant cell may be included in a plant tissue, a plant organ, and/or a whole plant in some embodiments. A plant cell in a tissue, organ, and/or whole plant may be adjacent, according to some embodiments, to one or more isogenic cells and/or one or more heterogenic cells. In some embodiments, a plant may include primary transformants and/or progeny thereof. A plant comprising a nucleic acid (e.g., a heterologous and/or expressible nucleic acid) comprising a nucleic acid sequence encoding an antimicrobial peptide may further comprise an expression control sequence operably linked to the nucleic acid, in some embodiments. A nucleic acid sequence encoding an antimicrobial peptide may be expressed, according to some embodiments, in a plant in one or more up to all (e.g., substantially all) organs, tissues, and/or cell types including, without limitation, stalks, leaves, roots, seeds, flowers, fruit, meristem, parenchyma, storage parenchyma, collenchyma, sclerenchyma, epidermis, mesophyll, bundle sheath, guard cells, protoxylem, metaxylem, phloem, phloem companion, and/or combinations thereof. In some embodiments, a nucleic acid and/or its gene product (e.g., an antimicrobial peptide) may be located in and/or translocated to one or more organelles (e.g., vacuoles, chloroplasts, mitochondria, plastids).

Preferred plants are citrus plants and include all plant parts and progeny, including, but not limited to fruit, juice, leaves, seeds, etc. that contain the expression vectors comprising spinach defensin genes.

IV. Methods

A. Transforming a Plant

The present disclosure relates, according to some embodiments, to methods for independent transformation of citrus (e.g., a native genome of a citrus plant). For example, a method may comprise independent transformation, using Agrobacterium tumefaciens (At), of the native genome of a citrus plant, such as an orange plant (Citrus sinensis). A transformation method may comprise contacting a nucleic acid comprising a SoD2 and/or SoD7 sequence (e.g., a SoD2 and/or SoD7 synthetic gene sequence) with a citrus plant according to some embodiments. A transformed plant (e.g., a transformed genome of a new orange cultivar) may independently contain, in some embodiments a sequence of a SoD2 gene and/or a SoD7 gene encoding microbial resistance not found within the native gene pool of the Citrus genus.

According to some embodiments, a transformed orange cultivar plant may comprise a peptide encoded by a SoD2 gene and/or a SoD7 gene. A transformed plant comprising a sequence of a SoD2 gene and/or a SoD7 gene and/or comprising a peptide encoded by a SoD2 gene and/or a SoD7 gene may display resistance to a range (e.g., a broad range) of bacterial and/or fungal pathogens in some embodiments. For example, a transformed plant comprising a sequence of a SoD2 gene and/or a SoD7 gene and/or comprising a peptide encoded by a SoD2 gene and/or a SoD7 gene may display resistance to bacterial canker (Xanthomonas axonopodis pv. citri) (Xac), and/or citrus Huanglongbing (ex greening) caused by Candidatus Liberibacter asiaticus (Las). See EXAMPLE section below.

B. Grafting

The present disclosure relates to grafting at least a portion of a first plant (e.g., a citrus plant) with at least a portion of a second plant (e.g., a citrus plant), according to some embodiments. A first plant may be in any desired condition including, without limitation, a healthy condition, a diseased condition, an injured condition, a stressed condition (e.g., heat, cold, water, and the like), and/or combinations thereof. A first plant may have any desired genotype including, without limitation, wild type, transgenic, mutant, and/or the like with respect to a gene and/or trait of interest.

A second plant may be in any desired condition including, without limitation, a healthy condition, a diseased condition, an injured condition, a stressed condition (e.g., heat, cold, water, and the like), and/or combinations thereof. A second plant may have any desired genotype including, without limitation, wild type, transgenic, mutant, and/or the like with respect to a gene and/or trait of interest. A first and/or a second plant may comprise at least one antimicrobial peptide and/or at least one nucleic acid comprising a sequence encoding at least one antimicrobial peptide. Where both a first plant comprises at least one antimicrobial peptide and/or at least one nucleic acid comprising a sequence encoding at least one antimicrobial peptide and a second plant comprises at least one antimicrobial peptide and/or at least one nucleic acid comprising a sequence encoding at least one antimicrobial peptide, it may be desirable for the first and second plants to have the same and/or different antimicrobial peptides and/or nucleic acids encoding antimicrobial peptides. Grafting may comprise cutting a portion of a first plant to form a fresh cut site, cutting a portion of a second plant to create a second cut site, and/or contacting a first cut site with a second cut site. A cut site may comprise at least one vascular bundle. Grafting may comprise forming a graft junction and/or, optionally, sealing the graft junction (e.g., by coating the periphery of the graft junction with one or more barrier materials).

C. Treating Plant Disease

The present disclosure relates, in some embodiments, to compositions, organisms, systems, and methods for preventing, ameliorating, and/or treating a plant disease (e.g., a citrus disease) and/or at least one symptom of a plant disease. For example, a method may comprise grafting at least a portion of a plant (e.g., a citrus plant) having a plant disease and/or expressing at least one symptom of a plant disease with at least a portion of a plant (e.g., a citrus plant) comprising an antimicrobial peptide. Examples of a plant disease include, without limitation, bacterial canker (Xanthomonas axonopodis pv. citri) (Xac), and/or citrus Huanglongbing (ex greening) caused by Candidatus Liberibacter asiaticus (Las). According to some embodiments, preventing, ameliorating, and/or treating a plant disease (e.g., a citrus disease) and/or at least one symptom of a plant disease may comprise treating and/or curing one or more devastating bacterial diseases of citrus. For example, plants comprising stably integrated SoD2 and SoD7 transgenes in expressible form may display resistance to, without limitation, bacterial canker (Xanthomonas axonopodis pv. citri) (Xac), and/or citrus Huanglongbing (ex greening) caused by Candidatus Liberibacter asiaticus (Las). Such resistance has been observed as described in the Examples below.

According to some embodiments, the present disclosure relates to compositions, organisms, systems, and methods for augmenting a plant's native resistance to and/or conferring on a plant resistance to a plant disease (e.g., a citrus disease). For example, a method may comprise contacting a plant with an antimicrobial peptide and/or an expressible nucleic acid comprising a nucleic acid sequence encoding an antimicrobial peptide. An expressible nucleic acid comprising a nucleic acid sequence encoding an antimicrobial peptide may be and/or comprise an expression cassette in some embodiments. Contacting may comprise, according to some embodiments, grafting at least a portion of a target plant with a plant comprising an antimicrobial peptide and/or an expressible nucleic acid comprising a nucleic acid sequence encoding an antimicrobial peptide. In some embodiments, contacting may comprise contacting at least a portion of a target plant with a vector (e.g., via Agrobacterium-mediated transformation) comprising an antimicrobial peptide and/or an expressible nucleic acid comprising a nucleic acid sequence encoding an antimicrobial peptide. Examples of a plant disease include, without limitation, bacterial canker (Xanthomonas axonopodis pv. citri) (Xac), and/or citrus Huanglongbing (ex greening) caused by Candidatus Liberibacter asiaticus (Las).

D. Making a Citrus-Expressible Antimicrobial Peptide

In some embodiments, the present disclosure relates to compositions, organisms, systems, and methods for forming a citrus-expressible nucleic acid comprising a nucleic acid sequence encoding at least one spinach-derived antimicrobial peptide. For example, a method may comprise identifying an amino acid sequence of an antimicrobial peptide of interest, reverse translating the amino acid sequence to produce a first nucleic acid sequence; codon-optimizing the first nucleic acid sequence for expression in citrus to produce a second nucleic acid sequence, and/or synthesizing a nucleic acid having the second nucleic acid sequence. A method may comprise, in some embodiments, covalently bonding a nucleic acid having the second nucleic acid sequence with one or more nucleic acids having expression control sequences that are operable in citrus in an operable orientation and/or position relative to the nucleic acid having the second nucleic acid sequence.

As will be understood by those skilled in the art who have the benefit of the instant disclosure, other equivalent or alternative pathogen resistant citrus compositions, organisms, systems, and methods can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of expression control sequences, coding sequences, linkers, and/or terminator sequences may be varied. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. For example, a composition, device, and/or system may be prepared and or used as appropriate for microbial and/or plant (e.g., with regard to sanitary, infectivity, safety, toxicity, biometric, and other considerations). Where desired, some embodiments of the disclosure may be practiced to the exclusion of other embodiments. For example, some polypeptide embodiments may be practiced to the exclusion of a particular amino acid sequence (e.g., SEQ ID NO:26) and/or some nucleic acid embodiments may be practiced to the exclusion of a particular nucleic acid sequence (e.g., SEQ ID NO:27).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

SPINCACH DEFENSIN EXAMPLES

Some specific example embodiments of the disclosure may be illustrated by one or more of the examples provided herein.

Plant materials (e.g., Citrus sinensis) were generally prepared for transformation as described by Yang et al., Plant Cell Reports (2000) 19:1203 et seq. Plasmid construction and bacterial strains were generally performed as described by Yang et al., Plant Cell Reports (2000) 19:1203 et seq. Agrobacterium co-culture and plant transformation were generally performed as described by Yang et al., Plant Cell Reports (2000) 19:1203 et seq. Selection and regeneration of transgenic shoots were generally performed as described by Yang et al., Plant Cell Reports (2000) 19:1203 et seq. Grafting of transgenic shoots were generally performed as described by Yang et al., Plant Cell Reports (2000) 19:1203 et seq. Southern and northern analysis were generally performed as described by Yang et al., Plant Cell Reports (2000) 19:1203 et seq.

### Example 1

### Expression in Citrus Trees

Table 1 illustrates specific example embodiments of nucleic acid sequences codon-optimized for citrus. Signal peptides and structural gene coding sequences shown are flanked on either side by specific restriction enzyme sites. These sequences were used to construct expression cassettes, vectors, and transformed Agrobacterium for preparation of transgenic plants.

TABLE 1
Example embodiments of specific nucleotide sequences of antimicrobial genes.
The nucleotide sequences were optimized for codon usage in Citrus.

| | Source of the | Antimicrobial genes specific nucleotide sequences. |
|---|---|---|
| | Optimized | The 5′ nucleotides include the cloning site and a |
| Antimicrobial | Synthetic Gene | preferred context for the start codon. The 3′ |
| Gene | (code) | nucleotides include the cloning site. |
| SoD2 | GenScript (07) | SEQ ID NO: 9 |
| | CODA (09) | SEQ ID NO: 11 |
| SoD7 | GenScript (08) | SEQ ID NO: 10 |
| | CODA (10) | SEQ ID NO: 12 |
| SoD2 | DNA 2.0 (11) | SEQ ID NO: 30 |
| SoD7 | DNA 2.0 (12) | SEQ ID NO: 31 |
| SoD2 + SoD7 | GenScript (13) | SEQ ID NOS: 9 and 10 |
| SoD2 + SoD7 | DNA 2.0 (16) | SEQ ID NO: 30 and 31 |
| no SP | | |

The following cultivars were selected for transformation: Orange: Hamlin (“04”), Rohde Red (“05”), and Marrs (“06”); Grapefruit: Ruby Red (“01”) and Rio Red; Carrizo Citrange (“CC”); Flying Dragon rootstock (“13” and “16”); Frost Eureka and Frost Lisbon (13” and “16”); Swingle rootstock (13” and “16”); and C22 rootstock. Constructs used for each cultivar are shown in Table 2 of U.S. application Ser. No. 13/751,936 (incorporated by reference). Citrus plants and citrus rootstock were transformed as described in example 7 and FIGS. 4-12 of U.S. application Ser. No. 13/751,936 (incorporated by reference).

### Example 2

### Canker Disease Resistance Assay

Canker disease resistance was assessed using a detached leaf assay generally as described by Francis M I et al., 2010, Eur J Plant Pathol 127:571-578. Briefly, detached immature leaves (˜75% expanded) were triple rinsed in sterile water to remove debris, sanitized by brief immersion in 70% ethanol followed by 0.5% sodium hypochloride, and again triple rinsed in sterile water. Sanitized leaves (3-4 per replicate×3 replicates) were infiltrated on their abaxial surface with an aqueous suspension of an Xcc strain isolated in Dade County Florida. Innoculated leaves were pressed on the surface of soft water agar plates, parafilm sealed, and incubated in an environmentally-controlled growth chamber. A large reduction in the size and number of lesion on the transgenic can be seen. See FIG. 13A and FIG. 13B of U.S. Ser. No. 13/751,936.

### Example 3

### Citrus Greening (HLB) Disease Resistance Assay by Grafting

FIG. 14 of U.S. Ser. No. 13/751,936 shows the result of graft inoculating non-transgenic ‘Rio Red’ (two trees on the left) or transgenic ‘Rio Red’ expressing SoD2 one tree on the right) with the citrus greening pathogen. A non-transgenic rootstock (Cleopatra mandarin) infected with HLB is used. Onto this rootstock several buds of transgenic ‘Rio Red’ are grafted and this is replicated. The same protocol is followed for non-transgenic buds of ‘Rio Red’. After 8 weeks, vigorous growth can be seen from the transgenic graft, where there is no growth on the controls.

### Example 4

### Citrus Greening (HLB) Disease Resistance Assay by Psyllid Inoculation

Resistance to bacterial infection and growth was assessed by two metrics. First, resistance was evaluated by the percentage of infection, namely the number of exposed plants that were infected. Second, a PCR-based method was used to amplify bacterial sequences. In this method, the relative degree of infection influences the number of PCR cycles required to produce detectable signal. For example a heavily infested plant might only require a few cycles while a plant with a low bacterial titer may require more cycles. In general, a plant that requires 30 or more cycles to observe detectable signal is regarded to be uninfected. Since some infections of citrus progress slowly, samples were collected for testing at 5 to 11 months after the time of first exposure and thereafter over a period of 6-9 months. The frequency of sample collection may vary from about every 45 days to about every 120 days. Ten to 15 replicates of each transgenic event plus non-transgenic controls are placed haphazardly in an insect proof green house that contains thousands' of psyllids carrying the citrus greening pathogen. The first PCR testing is done about five months after continuous exposure to psyllids. DNA extraction and PCR to detect the pathogen is essentially as described by Trey M S et al., 2006, Proc. Fla. State Hort. Soc. 119:89-93.

Propagation and Resistance of Generation 1, 2, 3, 4 and 5 is discussed in Examples 11-15 of U.S. Ser. No. 13/751,936.

### Example 5

### Expression of Defensin Constructs in Various Plants

Stable expression of defensin constructs comprising nucleic acid sequences codon-optimized for citrus has been confirmed in the following:

| Cultivar | Gene Code | # Events |
|---|---|---|
| Rio Red Grapefruit | 13 | 18 |
| Ruby Red Grapefruit | 11 and 12 | 12 |
| Hamlin Sweet Orange | 07, 08, 09, 10, 11, | over 86 |
| | 12, 13, and 16 | |
| Marrs Sweet Orange | 07 and 08 | 13 |
| Rohde Red Valencia Orange | 07, 08, 09, 10, 13 | over 48 |
| Frost Eureka Lemon | 13 and 16 | over 30 |
| Frost Lisbon Lemon | 13 and 16 | over 33 |
| C22 and Carrizo Citrange | 07, 08, 13 | 42 |
| Rootstocks | | |
| Flying dragon and Swingle | 13 | Multiple GUS+ |
| Rootstocks | | |

For all constructs, individual transformation events have been found spanning a range of expression levels from no expression (e.g., since Southern results demonstrate the gene is present, often in multiple copies, it may be that the transgene has been silenced) to low expression to high expression.

### Combination of Spinach Defensin Genes in CTV Vector

The present invention provides CTV vectors comprising spinach defensin genes that can be stably expressed and offer the plant having been transformed with the vector resistance to Citrus Huanglongbing (Ex Greening) Caused By Candidatus Liberibacter Asiaticus (Las) and Bacterial Canker Caused By (Xanthomonas Axonopodis pv. Citri) (Xac).

The CTV vector can be delivered to already existing trees or in the budwood used to create new trees. The vector containing the spinach defensin gene could be grafted into nursery trees with vector-infected budwood and the resulting trees could be grown by methods prior to the introduction of HLB. This approach avoids problems of juvenility of transgenic trees and the need for horticultural evaluation. The vector could be graft inoculated to existing trees in the field. The vector, because it can express any effective protein or peptide found by any laboratory is an alternative to genetic transformation of citrus. With the spinach defensin gene, it is possible that the vector could be used to treat HLB infected trees by graft inoculation into trees already infected with HLB.

### Example 1

### Testing of Orange Trees Infected with the CTV Vector Construct of the Invention

Test trees infected with the CTV vector construct of the invention will be planted in 16 rows×155 trees to provide 218 trees per acre. The trees will receive one of three different treatments One with SoD2 gene, one with SoD7 gene and one untreated (no viral vector). Thus, within each plot there will healthy plants and infected plants. The healthy plants are protected plants and have an increased tolerance due the CTV vector constructs of the present invention and the infected plants are cured by the CTV vector constructs of the present invention.

Optionally trees could be infected with a vector comprising both the SoD2 and the SoD7 gene. Another option is to treat trees with a CTV vector containing one or more spinach defensin genes (SoD2 and SoD7) in addition to a gene offering resistance to the psyllid.

### LIST OF REFERENCES

Ahlquist, P., French, R., Janda, M., Loesch-Fries, L. S., 1984. Multi component RNA plant virus infection derived from cloned viral cDNA. Proc. Natl. Acad. Sci. USA 8, 7066-7070.

Akbergenov, R, Zhanybekova, S., Kryldakov, R. V., Zhigailov, A., Polimbetova, N. S., Hohn, T., Iskakov, B. K., 2004. ARC-1, a sequence element complementary to an internal 18S rRNA segment, enhances translation efficiency in plants when present in the leader or intercistronic region of mRNAs. Nucleic Acids Res. 32, 239-247.

Allison, R. F., Dougherty, W. G., Parks, T. D., Willis, L., Johnston, R. E., Kelly, M., Armstrong, F. B., 1985. Biochemical analysis of the capsid protein gene and capsid protein of tobacco etch virus: N-terminal amino acids are located on the virion's surface. J. Virol. 147, 309-316.

Andrade, M., Sato, M., Uyeda, I., 2007. Two resistance modes to Clover yellow vein virus in pea characterized by a green fluorescent protein-tagged virus. Phytopathology 97, 544-550.

Ayllon, M. A., Gowda, S., Satyanarayana, T., Karasev, A. V., Adkins, S., Mawassi, M., Guerri, J., Moreno, P., Dawson, W. O., 2003. Effects of modification of the transcription initiation site context on citrus tristeza virus subgenomic RNA synthesis. J. Virol. 77, 9232-9243.

Balvay, L., Soto Rifo, R., Ricci, E. P., Decimo, D., Ohlmann, T., 2009. Structural and functional diversity of viral IRESes. Biochim. Biophys. Acta 1789, 542-557.

Baulcombe, D. C., Chapman, S., Santa Cruz, S., 1995. Jellyfish green fluorescent protein as a reporter for virus infections. Plant J. 7, 1045-1053. [0130] Beauchemin, C., Bougie, V., Laliberte, J. F., 2005. Simultaneous production of two foreign proteins from a potyvirus-based vector. Virus Res. 112, 1-8.

Brisson, N., Paszkowski, J., Penswick, J. R., Gronenborn, B., Potrykus, I., Hohn, T., 1984. Expression of a bacterial gene in plants by using a viral vector. Nature 310, 511-514.

Canizares, C. M., Navas-Castillo, J., Moriones, E., 2008. Multiple suppressors of RNA silencing encoded by both genomic RNAs of the crinivirus, Tomato chlorosis virus. Virology 379, 168-174.

Canizares, M. C., Nicholson, L., Lomonossoff, G. P., 2005 Use of viral vectors for vaccine production in plants. Immunol. Cell Biol. 83, 263-270.

Canto, T., Prior, D. A. M., Hellwald, K.-H., Oparka, K. J., Palukaitis, P., 1997. Characterization of cucumber mosaic virus IV. Movement protein and coat protein are both essential for cell-to-cell movement of cucumber mosaic virus. Virology 237, 237-248.

Carrasco, P., Daros, J. A., Agudelo-Romero, P., Elena, S. F., 2007. A real-time RT-PCR assay for quantifying the fitness of tobacco etch virus in competition experiments. J. Virol. Methods 139, 181-188. [0136] Carrington, J. C., Cary, S. M., Parks, T. D. and Dougherty, W. G., 1989. A second proteinase encoded by a plant potyviral genome. EMBO J. 8, 365-370.

Carrington, J. C., Dougherty, W. G., 1988. A viral cleavage site cassette: Identification of amino acid sequences required for tobacco etch virus polyprotein processing. Proc. Natl. Acad. Sci. USA 85, 3391-3395.

Chalfie, M., Tu, Y., Euskirchen, G., Ward, W. W., Prasher, D. C., 1994. Green fluorescent protein as a marker for gene expression. Science 263, 802-805.

Chapman, S., Kavanagh, T., Baulcombe, D., 1992. Potato virus X as a vector for gene expression in plants. Plant J. 2, 549-557.

Chiba, M., Reed, J. C., Prokhnevsky, A. I., Chapman, E. J., Mawassi, M., Koonin, E. V., Carrington, J. C., Dolja, V. V., 2006. Diverse suppressors of RNA silencing enhance agroinfection by a viral replicon. Virology 346, 7-14.

Crameri, A., Whitehorn, E. A., Tate, E., Stemmer, W. P., 1996. Improved green fluorescent protein by molecular evolution using DNA shuffling. Nat. BioTechniques 14, 315-319.

Dawson, W. O., Bubrick, P., Grantham, G. L. 1988. Modifications of the tobacco mosaic virus coat protein gene affect replication, movement, and symptomatology. Phytopathology 78, 783-789.

Dawson, W. O., Lewandowski, D. J., Hilf, M. E., Bubrick, P., Raffo, A. J., Shaw, J. J., Grantham, G. L. Desjardins, P. R., 1989. A tobacco mosaic virus-hybrid expresses and loses an added gene. Virology 172, 285-292.

Deleris, A., Gallego-Bartolome, J., Bao, J., Kasschau, K. D., Carrington, J. C., Voinnet, 0., 2006. Hierarchical action and inhibition of plant Dicer-like proteins in antiviral defense. Science 313, 68-71.

Dietrich, C., Maiss, E., 2003. Fluorescent labeling reveals spatial separation of potyvirus populations in mixed infected Nicotiana benthamiana plants. J. Gen. Virol. 84, 2871-2876.

Dolja, V. V., Hong, J., Keller, K. E., Martin, R. R., Peremyslov, V. V., 1997. Suppression of potyvirus infection by co-expressed closterovirus protein. Virology 234, 243-252.

Dolja, V. V., McBride, H. J., Carrington, J. C., 1992. Tagging of plant potyvirus replication and movement by insertion of beta-glucuronidase into the viral polyprotein. Proc. Natl. Acad. Sci. USA 89, 10208-10212.

Donson, J., Kearney, C. M., Hilf, M. E., Dawson, W. O. 1991. Systemic expression of a bacterial gene by a tobacco mosaic virus-based vector. Proc. Natl. Acad. Sci. USA. 88, 7204-7208.

Dorokhov, Y. L., Skulachev, M. V., Ivanov, P. A., Zvereva, S. D., Tjulkina, L. G., Merits, A., Gleba, Y. Y., Hohn, T., Atabekov, J. G., 2002. Polypurine (A)-rich sequences promote cross-kingdom conservation of internal ribosome entry. Proc. Natl. Acad. Sci. USA. 99, 5301-5306.

Edelstein, M. L., Abedi, M. R. Wixon, J., 2007. Gene therapy clinical trials worldwide to 2007—an update. J. Gene Med. 9, 833-842.

Fernandez-Miragall, 0., Lopez de Quinto, S., Martinez-Salas, E., 2009. Relevance of RNA structure for the activity of picornavirus IRES elements. Virus Res. 139, 172-182.

Fitzgerald, K. D., Semler, B. L., 2009. Bridging IRES elements in mRNAs to the eukaryotic translation apparatus. Biochim. Biophys. Acta 1789, 518-528.

Folimonov, A. S., Folimonova, S. Y., Bar-Joseph, M., Dawson, W. O., 2007. A stable RNA virus-based vector for citrus trees. Virology 368, 205-216.

Folimonova, S. Y., Folimonov, A. S., Tatineni, S., Dawson, W. O., 2008. Citrus tristeza virus: survival at the edge of the movement continuum. J. Virol. 82, 6546-6556.

Folimonova, S. Y., Robertson, C. J., Shilts, T., Folimonov, A. S., Hilf, M. E., Garnsey, S. M. Dawson, W. O., 2010. Infection with strains of Citrus tristeza virus does not exclude super infection by other strains of the virus. J. Virol. 84, 1314-1325.

French, R., Janda, M., Ahlquist, P., 1986. Bacterial gene inserted in an engineered RNA virus: efficient expression in monocotyledonous plant cells. Science 231, 1294-97

Futterer, J., Bonneville, J. M., Hohn, T., 1990. Cauliflower mosaic virus as a gene expression vector for plants. Physiol. Plant. 79, 154-157.

Gallie, D. R., 2001. Cap-independent translation conferred by the 5′ leader of tobacco etch virus is eukaryotic initiation factor 4G dependent. J. Virol. 75, 12141-12152.

Gallie, D. R., Tanguay, R. L., Leathers, V., 1995. The tobacco etch viral 5′ leader and poly(A) tail are functionally synergistic regulators of translation. Gene 165, 233-238.

Garnsey, S. M., Gonsalves, D., Purcifull, D. E., 1977. Mechanical transmission of citrus tristeza virus. Phytopathology 67, 965-968.

Garnsey, S. M., Cambra, M., 1991. Enzyme-linked immunosorbent assay (ELISA) for citrus pathogens. In: Roistacher, C. N. (Ed.), Graft-Transmissible Diseases of Citrus, Handbook for Detection and Diagnosis. FAO, Rome, pp. 193-216.

Garnsey, S. M., Henderson C. T., 1982. Extraction, centrifugation, and assay techniques for purification of intact citrus tristeza virus. Workshop on Plant Virus Detection, Agric. Exp. Stn., University of Puerto Rico, Rio Piedras, Mar. 29-Apr. 2, 1982, 106-112.

Giritch, A., Marillonnet, S., Engler, C., van Eldik, G., Botterman, J., Klimyuk, V., Gleba, Y., 2006. Rapid high-yield expression of full-size IgG antibodies in plants coinfected with noncompeting viral vectors. Proc. Natl. Acad. Sci. USA 103, 14701-14706.

Gleba, Y., Klimyuk, V., Marillonnet, S., 2007. Viral vectors for the expression of proteins in plants. Curr. Opin. Biotechnol. 18, 134-141.

Gopinath, K., Wellink, J., Porta, C., Taylor, K. M., Lomonossoff, G. P., van Kammen, A., 2000. Engineering cowpea mosaic virus RNA-2 into a vector to express heterologous proteins in plants. Virology 267, 159-173.

Gowda, S., Satyanarayana, T., Ayllon, M. A., Albiach-Marti, M. R., Mawassi, M., Rabindran, S., Garnsey, S. M., Dawson, W. O., 2001. Characterization of the cis-acting elements controlling subgenomic mRNAs of citrus tristeza virus: production of positive- and negative-stranded 3′-terminal and positive-stranded 5′-terminal RNAs. Virology 286 1, 134-151.

Gowda, S., Satyanarayana, T., Davis, C. L., Navas-Castillo, J., Albiach-Marti, M. R., Mawassi, M., Valkov, N., Bar-Joseph, M., Moreno, P., Dawson, W. O., 2000. The p20 gene product of Citrus tristeza virus accumulates in the amorphous inclusion bodies. Virology 274, 246-254.

Gowda, S., Satyanarayana, T., Robertson, C. J., Garnsey, S. M., Dawson, W. O., 2005. Infection of citrus plants with virions generated in Nicotiana benthamiana plants agroinfiltrated with a binary vector based Citrus tristeza virus, p. 23-33. In M. E. Hilf, N. Duran-Vila, and M. A. Rocha-P\tilde{e}na (eds.), Proceedings of the 16th Conference of the International Organization of Citrus Virologists. IOCV, Riverside, Calif., 728 USA.

Grdzelishvili, V. Z., Chapman, S. N., Dawson, W. O., Lewandowski, D. J., 2000. Mapping of the Tobacco mosaic virus movement protein and coat protein subgenomic RNA promoters in vivo. Virology 275, 177-192.

Gronenborn, B., Gardner, R. C., Schaefer, S., Shepherd, R. J., 1981. Propagation of foreign DNA in plants using cauliflower mosaic virus as vector. Nature 294, 773-76.

Hagiwara, Y., Peremyslov, V. V., Dolja, V. V., 1999. Regulation of closterovirus gene expression examined by insertion of a self-processing reporter and by northern hybridization. J. Virol. 73, 7988-7993.

Hilf, M. E., Karasev, A. V., Pappu, H. R., Gumpf, D. J., Niblett, C. L., Garnsey, S. M., 1995. Characterization of citrus tristeza virus subgenomic RNAs in infected tissue. Virology 208, 576-582.

Horton, R. M., Hunt, H. D., Ho, S. N., Pullen, J. R., Pease, L. B., 1989. Engineering hybrid genes without the use of restriction enzymes: gene splicing by overlap extension. Gene 77, 61-68.

Hu, C. D., Chinenov, Y., Kerppola, T. K., 2002. Visualization of interactions among bZIP and Rd family proteins in living cells using bimolecular fluorescence complementation. Molecular Cell 9, 789-798.

Ion-Nagy, L., Lansac, M., Eyquard, J. P., Salvador, B., Garcia, J. A., Le Gall, O., Hernould, M., Schurdi-Levraud, V., Decroocq, V., 2006. PPV long-distance movement is occasionally permitted in resistant apricot hosts. Virus Res. 120, 70-78.

Ivanov, P. A., Karpova, O. V., Skulachev, M. V., Tomashevskaya, O. L., Rodionova, N. P., Dorokhov, Y. L., Atabekov, J. G., 1997. A tobamovirus genome that contains an internal ribosome entry site functional in vitro. Virology 232, 32-43.

Karasev, A. V., 2000. Genetic diversity and evolution of closteroviruses. Annu. Rev. Phytopathol. 38, 293-324.

Karasev, A. V., Boyko, V. P., Gowda, S., Nikolaeva, O. V., Hilf, M. E., Koonin, E. V., Niblett, C. L., Cline, K., Gumpf, D. J., Lee, R. F., Garnsey, S. M., Lewandowski, D. J., Dawson, W. O., 1995. Complete sequence of the citrus tristeza virus RNA genome. Virology 208, 511-520.

Karasev, A. V., Nikolaeva, O. V., Mushegian, A. R., Lee, R. F. Dawson, W. O., 1996. Organization of the 3′-terminal half of beet yellow stunt virus genome and implications for the evolution of closteroviruses. J. Virol. 221, 199-207.

Kasschau, K. D., Xie, Z., Allen, E., Llave, C., Chapman, E. J., Krizan, K. A., and Carrington, J. C., 2003. P1/HC-Pro, a viral suppressor of RNA silencing, interferes with Arabidopsis development and miRNA function. Dev. Cell 4, 205-217.

Kawakami, S., Watanabe, Y., Beachy, R. N., 2004. Tobacco mosaic virus infection spreads cell to cell as intact replication complexes. Proc. Natl. Acad. Sci. USA 101, 6291-6296.

Kelloniemi, J., Makinen, K., Valkonen, J. P. T., 2008. Three heterologous proteins simultaneously expressed from a chimeric potyvirus: infectivity, stability and the correlation of genome and virion lengths. Virus Res. 135, 282-291.

Kneller, E. L., Rakotondrafara, A. M., Miller, W. A., 2006. Cap independent translation of plant viral RNAs. Virus Res. 119, 63-75.

Koh, D. C., Wong, S. M., Liu, D. X., 2003. Synergism of the 3′-untranslated region and an internal ribosome entry site differentially enhances the translation of a plant virus coat protein. J. Biol. Chem. 278, 20565-20573.

Lehto, K., and Dawson, W. O., 1990. Replication, stability, and gene expression of tobacco mosaic virus mutants with a second 30K ORF. Virology 175, 30-40.

Lewandowski, D. J. and Dawson, W. O., 1998. Deletion of internal sequences results in Tobacco mosaic virus defective RNAs that accumulate to high levels without interfering with replication of the helper virus. Virology 251, 427-437.

Lico, C., Chen, Q., Santi, L., 2008. Viral vectors for production of recombinant proteins in plants. J. Cell Physiol. 216, 366-377.

Liu, Y. P., Peremyslov, V. V., Medina, V., Dolja, V. V. 2009. Tandem leader proteases of Grapevine leafroll-associated virus 2: host-specific functions in the infection cycle. Virology 383, 291-299.

Lopez, C., Navas-Castillo, J., Gowda, S., Moreno, P., Flores R., 2000. The 23-kDa protein coded by the 3′-terminal gene of citrus tristeza virus is an RNA-binding protein. Virology 269, 462-470.

Lu, R., Folimonov, A., Shintaku, M., Li, W. X., Falk, B. W., Dawson, W. O., Ding, S. W., 2004. Three distinct suppressors of RNA silencing encoded by a 20-kb viral RNA genome. Proc. Natl. Acad. Sci. USA 101, 15742-15747.

Lucy, A. P., Guo, H. S., L.sub.1, W. X., Ding, S. W., 2000. Suppression of post-transcriptional gene silencing by a plant viral protein localized in the nucleus. EMBO J. 19, 1672-1680.

Marton, I., Zuker, A., Shklarman, E., Zeevi, V., Tovkach, A., Roffe, S., Ovadis, M., Tzfira, T., Vainstein, A., 2010. Nontransgenic genome modification in plant cells. Plant Physiol. 154, 1079-1087.

Masoumi, A., Hanzlik, T. N., Christian, P. D., 2003. Functionality of the 59- and intergenic IRES elements of cricket paralysis virus in a range of insect cell lines, and its relationship with viral activities. Virus Res. 94, 113-120.

Masuta, C., Yamana, T., Tacahashi, Y., Uyeda, I., Sato, M., Ueda, S., Matsumura, T., 2000. Development of clover yellow vein virus as an efficient, stable gene-expression system for legume species. Plant J. 23, 539-546.

Navas-Castillo, J., Albiach-Marto A, M. R., Gowda, S., Hilf, M. E., Garnsey, S. M., Dawson, W. O., 1997. Kinetics of accumulation of citrus tristeza virus RNAs. Virology 228, 92-97.

Niepel, M., Gallie, D. R., 1999. Identification and characterization of the functional elements within the tobacco etch virus 5′ leader required for cap-independent translation. J. Virol. 73, 9080-9088.

Padgett, H. S., Epel, B. L., Heinlein, M. H., Watanabe, Y., Beachy, R. N. 1996. Distribution of tobamovirus movement protein in infected cells and implications for cell-to-cell spread of infection. Plant J. 10, 1079-1099.

Pappu, H. R., Karasev, A. V., Anderson, E. J., Pappu, S. S., Hilf, M. E., Febres, V. J., Eckloff, R. M. G., McCaffery, M., Boyko, V., Gowda, S., Dolia, V. V., Koonin, E. V., Gumpf, D. J., Cline, K. C., Garnsey, S. M., Dawson, W. O., Lee, R. F., Niblett, C. L., 1994. Nucleotide sequence and organization of eight 3′ open reading frames of the Citrus tristeza closterovirus genome. Virology 199, 35-46.

Peremyslov, V. V., Hagiwara, Y., Dolja, V. V., 1999. HSP70 homolog functions in cell-to-cell movement of a plant virus. Proc. Natl. Acad. Sci. U.S.A. 96, 14771-14776.

Prokhnevsky, A. I., V. V. Peremyslov, V. V., Napuli, A. J., Dolja, V. V., 2002. Interaction between long-distance transport factor and Hsp70-related movement protein of beet yellows virus. J. Virol. 76, 11003-11011.

Ratcliff, F., MacFarlane, S., Baulcombe, D. C., 1999. Gene silencing without DNA: RNA-mediated cross protection between viruses. Plant Cell, 11, 1207-1215.

Roberts, A. G., Santa Cruz, S., Roberts, I. M., Prior, D. A. M., Turgeon, R., Oparka, K. J., 1997. Phloem unloading in sink leaves of Nicotiana benthamiana comparison of a fluorescent solute with a fluorescent virus. Plant Cell 9, 1381-1396.

Roberts, L. O., Groppelli, E., 2009. An atypical IRES within the 50 UTR of a dicistrovirus genome. Virus Res. 139, 157-165.

Robertson, C. J., Garnsey, S. M., Satyanarayana, T., Folimonova, S., Dawson, W. O., 2005. Efficient infection of citrus plants with different cloned constructs of Citrus tristeza virus amplified in Nicotiana benthamiana protoplasts. Proc. 16th Conf. IOCV. IOCV, Riverside, Calif., pp. 187-195.

Roy, G., Weisburg, S., Rabindran, S., Yusibov, V., 2010. A novel two-component Tobacco mosaic virus-based vector system for high-level expression of multiple therapeutic proteins including a human monoclonal antibody in plants. Virology 405, 93-99.

Sanchez-Navarro, J. A., Miglino, R., Ragozzino, A., and Bol, J. F., 2001. Engineering of Alfalfa mosaic virus RNA 3 into an expression vector. Arch. Virol. 146, 923-939.

Sato, M., Masuta, C., Uyeda, I., 2003. Natural resistance to Clover yellow vein virus in beans controlled by a single recessive locus. Mol. Plant. Microbe Interact. 16, 994-1002.

Satyanarayana, T., Bar-Joseph, M., Mawassi, M., Albiach-Marti, M. R., Ayllon, M. A., Gowda, S., Hilf, M. E., Moreno, P., Garnsey, S. M., Dawson, W. O., 2001. Amplification of Citrus tristeza virus from a cDNA clone and infection of citrus trees. Virology 280, 87-96.

Satyanarayana, T., Gowda, S., Ayllon, M. A., Albiach-Marti, M. R., Dawson, W. O., 2002a. Mutational analysis of the replication signals in the 3′-non translated region of Citrus tristeza virus. Virology 300, 140-152.

Satyanarayana, T., Gowda, S., Ayllon, M. A., Albiach-Marti, M. R., Rabindram, R., Dawson, W. O. 2002b. The p23 protein of Citrus tristeza virus controls asymmetrical RNA accumulation. J. Virol. 76, 473-483.

Satyanarayana, T., Gowda, S., Ayllon, M. A., Dawson, W. O., 2003. Frame shift mutations in infectious cDNA clones of Citrus tristeza virus: a strategy to minimize the toxicity of viral sequences to Escherichia coli. Virology 313, 481-491.

Satyanarayana, T., Gowda, S., Ayllon, M. A., Dawson, W. O., 2004. Closterovirus bipolar virion: evidence for initiation of assembly by minor coat protein and its restriction to the genomic RNA 5′ region. Proc. Natl. Acad. Sci. USA 101, 799-804.

Satyanarayana, T., Gowda, S., Boyko, V. P., Albiach-Marti, M. R., Mawassi, M., Navas-Castillo, J., Karasev, A. V., Dolja, V., Hilf, M. E., Lewandowski, D. J., Moreno, P., Bar-Joseph, M., Garnsey, S. M., Dawson, W. O., 1999. An engineered closterovirus RNA replicon and analysis of heterologous terminal sequences for replication. Proc. Natl. Acad. Sci. USA 96, 7433-7438.

Satyanarayana, T., Gowda, S., Mawassi, M., Albiach-Marti, M. R., Ayllon, M. A., Robertson, C., Garnsey, S. M., Dawson, W. O., 2000. Closterovirus encoded HSP70 homolog and p61 in addition to both coat proteins function in efficient virion assembly. Virology 278, 253-265.

Shivprasad, S., Pogue, G. P., Lewandowski, D. J., Hidalgo, J., Donson, J., Grill, L. K., Dawson, W. O., 1999. Heterologous sequences greatly affect foreign gene expression in tobacco mosaic virus-based vectors. Virology 255, 312-323.

Siegel, A., 1983. RNA viruses as cloning vehicles. Phytopathology 73,775.

Siegel, A., 1985. Plant-virus-based vectors for gene transfer may be of considerable use despite a presumed high error frequency during RNA synthesis. Plant Mol. Biol. 4, 327-29.

Takahashi, T., Sugawara, T., Yamatsuta, T., Isogai, M., Natsuaki, T., Yoshikawa, N., 2007. Analysis of the spatial distribution of identical and two distinct virus populations differently labeled with cyan and yellow fluorescent proteins in coinfected plants. Phytopathology 97, 1200-1206

Takamatsu, N., Ishikawa, M., Meshi, T., Okada, Y., 1987. Expression of bacterial chloramphenicol acetyl transferase gene in tobacco plants mediated by TMV-RNA. EMBO J. 6, 307-311.

Tatineni, S., Gowda, S., Dawson W. O., 2010. Heterologous minor coat proteins of Citrus tristeza virus strains affect encapsidation, but the coexpression of HSP70h and p61 restores encapsidation to wild-type levels. Virology 402, 262-270.

Tatineni, S., McMechan A. J., Hein G. L., French R., 2011. Efficient and stable expression of GFP through Wheat streak mosaic virus-based vectors in cereal hosts using a range of cleavage sites: Formation of dense fluorescent aggregates for sensitive virus tracking. Virology 410, 268-281.

Tatineni, S., Robertson, C. J., Garnsey, S. M., Bar-Joseph, M., Gowda, S., Dawson, W. O., 2008. Three genes of Citrus tristeza virus are dispensable for infection and movement throughout some varieties of citrus trees. Virology 376, 297-307.

Toth, R. L., Chapman, S., Carr, F., Santa Cruz, S., 2001. A novel strategy for the expression of foreign genes from plant virus vectors. FEBS Lett. 489, 215-219.

Turpen, T. H., Turpen, A. M., Weinzettl, N., Kumagai, M. H., Dawson, W. O., 1993. Transfection of whole plants from wounds inoculated with Agrobacterium tumefaciens containing cDNA of tobacco mosaic virus. J. Virol. Meth. 42, 227-239.

Van Vloten-Doting, L., 1983. Advantages of multiple partite genomes of single-stranded RNA plant viruses in nature, for research and genetic engineering. Plant Mol. Biol. 1, 55-60.

Van Vloten-Doting, L., Bol, J. F., Cornelissen, B., 1985. Plant virus-based vectors for gene transfer will be of limited use because of the high error frequency during viral RNA synthesis. Plant Mol. Biol. 4, 323-326.

Verch, T., Yusibov, V., Koprowski, H., 1998. Expression and assembly of a full-length monoclonal antibody in plants using a plant virus vector. J. Immunol. Methods 220, 69-75.

Verver, J., Wellink, J., Van Lent, J., Gopinath, K., and Van Kammen, A., 1998. Studies on the movement of cowpea mosaic virus using the jellyfish green fluorescent protein. Virology 242, 22-27.

Woolaway, K. E., Lazaridis, K., Belsham, G. J., Carter, M. J., Robert, L. O., 2001. The 5′ untranslated region of rhopalosiphum padi virus contains an internal ribosome entry site which functions efficiently in mammalian, plant, and insect translation system. J. Virol. 75, 10244-10249.

1. A CTV viral vector engineered to comprise at least one gene cassette comprising a polynucleotide encoding at least one spinach defensin peptide, the CTV viral vector engineered such that the gene cassette is positioned at CTV genome regions p13-p20, p20-p2, p23-3′NTR, or positioned in place of p13, or positioned after p13 or p23 or between the minor coat protein and the coat protein.

2. The CTV viral vector of claim 1, wherein the at least one gene cassette further comprises a subgenomic mRNA controller element positioned upstream of said polynucleotide encoding at least one spinach defensin peptide.

3. A plant, plant part or plant progeny comprising at least one cell transfected with the CTV viral vector of claim 1.

4. A method of infecting a tree to express at least one spinach defensin peptide, said method comprising transfecting at least one cell of said tree with the CTV viral vector of claim 1.

5. The CTV viral vector of claim 1 engineered to comprise a gene cassette comprising a polynucleotide encoding at least one spinach defensin peptide and IRES sequence conjugated thereto.

6. A CTV viral vector engineered to comprise a gene cassette comprising a first polynucleotide sequence encoding a first spinach defensin peptide, a protease with cleavage sites engineered on each end, and a second polynucleotide sequence encoding a second spinach defensin peptide, wherein the spinach defensin peptides are the same or different.

7. The CTV viral vector of claim 6, wherein said protease is positioned between said first polynucleotide sequence encoding spinach defensin peptide and said second polynucleotide sequence encoding said second spinach defensin peptide.

8. The CTV viral vector of claim 6, wherein the gene cassette further comprises a subgenomic mRNA controller element positioned upstream of said first polynucleotide encoding the first spinach defensin peptide.

9. The CTV viral vector of claim 6, wherein said polynucleotide further encodes a protease and protease recognition sites between said first and second spinach defensin polypeptide sequence.

10. The CTV viral vector of claim 6, wherein said gene cassette is positioned at CTV genome regions p13-p20, p20-p2, p23-3′NTR, or positioned in place of p13, or positioned after p13 or p23 or between the minor coat protein and the coat protein.

11. A plant, plant part or plant progeny comprising at least one cell transfected with a CTV viral vector of claim 6.

12. A CTV viral vector engineered to comprise a gene cassette comprising a polynucleotide sequence encoding at least one spinach defensin peptide and a protease with cleavage sites engineered on each end fused in the same reading frame with a viral protein.

13. The CTV viral vector of claim 12, wherein said protease is positioned between said viral protein and said spinach defensin peptide.

14. The CTV viral vector of claim 12, wherein said gene cassette is fused to a endogenous CTV gene such that translation of an endogenous gene and said polynucleotide sequence encoding a spinach defensing peptide occurs together.

15. The CTV viral vector of claim 14, wherein said endogenous gene is p13, p20, or p23.

16. A method of infecting a tree to express a first and second spinach defensin peptide, said method comprising transfecting at least one cell of said tree with the CTV viral vector of claim 12.

17. A CTV viral vector engineered to comprise a first gene cassette comprising a first polynucleotide sequence encoding a first spinach peptide and a first controller element upstream of said first polynucleotide sequence; and a second gene cassette comprising a second polynucleotide sequence encoding a second spinach peptide and a second control element upstream of said second polynucleotide sequence.

18. The CTV viral vector of claim 17, wherein said first and second gene cassettes are positioned sequentially at the same location on said CTV viral vector.

19. The CTV viral vector of claim 17, wherein said first and second gene cassettes are positioned at two separate locations on said CTV viral vector.

20. The CTV viral vector of claim 18, wherein said gene cassettes are positioned at CTV genome regions p13-p20, p20-p2, p23-3′NTR, or positioned in place of p13, or positioned after p13 or p23 or between the minor coat protein and the coat protein.

21. The CTV viral vector of claim 19, wherein said one or both gene cassettes are inserted in place of an endogenous gene, and said second gene cassette is positioned at a location separate to said first gene cassette.

22. A plant, plant part, or plant progeny comprising at least one cell transfected with a CTV viral vector of claim 17.

23. A method of infecting a tree to express a first spinach defensin peptide, said method comprising transfecting at least one cell of said tree with the CTV viral vector of claim 17.

24. The vector of claim 1, wherein said CTV viral vector is engineered to comprise multiple gene cassettes located at one or multiple positions.

25. The vector of claim 24, wherein said CTV viral vector comprises at least two gene cassettes at one position and at least one gene cassette at a different location.

26. The method of claim 4, wherein said CTV viral vector is engineered to comprise multiple gene cassettes located at one or multiple positions.

27. The vector of claim 17, further comprising a third gene cassette comprising a third heterologous polynucleotide sequence encoding a third spinach defensin peptide and a third controller element upstream of said third heterologous polynucleotide sequence.

28. The vector of claim 27, further comprising a fourth gene cassette comprising a fourth heterologous polynucleotide sequence encoding a fourth spinach defensin peptide and a fourth controller element upstream of said fourth heterologous sequence.

29. The vector of claim 27, wherein said third gene cassette is positioned at the same location or different location on said CTV vector relative to said first and second gene cassettes.

30. The vector of claim 28, wherein said third and fourth gene cassettes are positioned sequentially at the same location on said CTV viral vector.

31. The CTV viral vector of claim 28, wherein said third and fourth gene cassettes are positioned at two separate locations on said CTV viral vector.
